(19) United States
(12) Patent Application Publication
(10) Pub. No.: US 2026/0200970 A1
(43) Pub. Date: Jul. 16, 2026

(54) PROCESS FOR MAKING AURISTATIN CONTAINING COMPOUNDS

(71) Applicant: Seagen Inc., Bothell, WA (US)

(72) Inventors: Ian ANDREWS, Collegeville, PA (US); John L. WOODARD, Collegeville, PA (US); Anthony Joseph NOCKET, Collegeville, PA (US); John Anthony KOWALSKI, Collegeville, PA (US); Greg Alan ERICKSON, Collegeville, PA (US); Danny Emilio MANHENO, Collegeville, PA (US); Michel JOURNET, Collegeville, PA (US); Qiaogong SU, Collegeville, PA (US); Christopher REIHER, Collegeville, PA (US); Kenneth Lamar ARRINGTON, Collegeville, PA (US)

(73) Assignee: Seagen Inc., Bothell, WA (US)

(21) Appl. No.: 19/136,073

(22) PCT Filed: Dec. 8, 2023
(86) PCT No.: PCT/US2023/083126

§ 371 Date: Jun. 5, 2025

Related U.S. Application Data
(60) Provisional application No. 63/431,637, filed on Dec. 9, 2022.

(51) Int. Cl. C07K 1/113

(52) U.S. Cl. CPC C07K 1/113

(57) ABSTRACT

Processes for making auristatin compounds such as maleimidocaproyl monomethyl Auristatin F (1), Formula (1) are described herein.

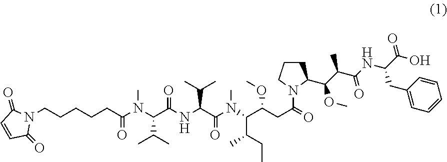 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@H](C(C)C)N(C)C(=O)CCCCCN1C(=O)C=CC1=O)C(C)C]

### CROSS-REFERENCE TO RELATED APPLICATIONS

This application claims priority to U.S. Provisional Application Ser. No. 63/431,637, filed Dec. 9, 2022, which is hereby incorporated herein by reference in its entirety.

### FIELD OF THE INVENTION

This disclosure relates processes and methods for making auristatin containing compounds.

### BACKGROUND

Multiple myeloma (MM), also known as myeloma is a rare type of cancer affecting plasma cells. Relapsed and refractory multiple myeloma (RRMM) is a condition in which a myeloma patient that has been treated for multiple myeloma, has the cancer return or the patient has not responded to any treatment (https://www.medicalnewstoday.com/articles/relapsed-refractory-multiple-myeloma). Blenrep® (belantamab mafodotin) is a humanized monoclonal antibody against the B-cell maturation antigen (BCMA) that is conjugated with potent cytotoxic agent, maleimidocaproyl monomethyl auristatin F (mcMMAF). This first-in-class medication was approved by the FDA in 2020 for the treatment of relapsed and refractory multiple myeloma. All references cited herein, including patent applications, patent publications, and scientific literature, are herein incorporated by reference in their entirety, as if each individual reference were specifically and individually indicated to be incorporated by reference.

### SUMMARY

Disclosed are processes and methods for making auristatin containing compounds such as peptide maleimidocaproyl monomethyl Auristatin F (1).

In a first aspect, a process for making is maleimidocaproyl monomethyl Auristatin F described herein, the process comprising (i) converting compound

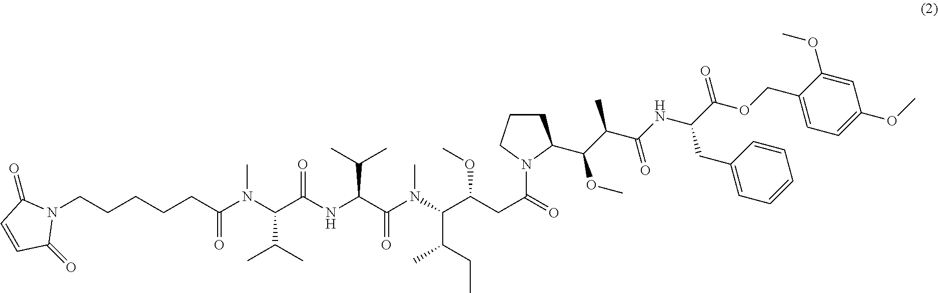 [SMILES: CC[C@H](C)[C@@H]([C@@H](CC(=O)N1CCC[C@H]1[C@H](OC)[C@@H](C)C(=O)N[C@@H](Cc1ccccc1)C(=O)OCc1ccc(OC)cc1OC)OC)N(C)C(=O)[C@@H](NC(=O)[C@H](C(C)C)N(C)C(=O)CCCCCN1C(=O)C=CC1=O)C(C)C]

to maleimidocaproyl monomethyl Auristatin F in the presence of toluene and 2-methyltetrahydrofuran, (ii) an aqueous workup, and (iii) chromatographic purification.

In a second aspect, a process for making

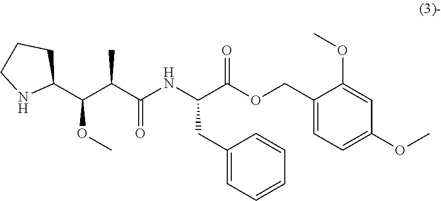 [SMILES: COc1ccc(COC(=O)[C@H](Cc2ccccc2)NC(=O)[C@H](C)[C@@H](OC)[C@@H]2CCCN2)c(OC)c1]

HCl is provided, the process comprising (i) reacting

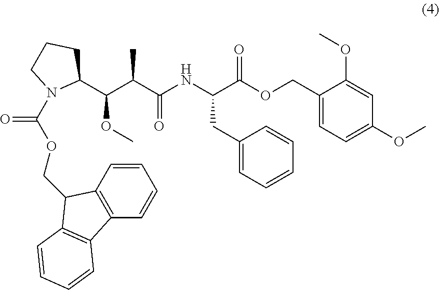 [SMILES: COc1ccc(COC(=O)[C@H](Cc2ccccc2)NC(=O)[C@H](C)[C@@H](OC)[C@@H]2CCCN2C(=O)OCC2c3ccccc3-c3ccccc32)c(OC)c1]

in the presence of a catalytic amount of DBU, and (ii) isolating

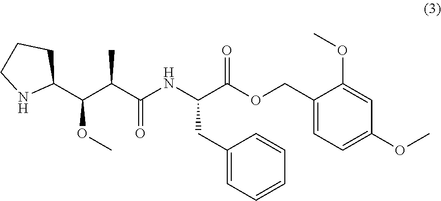 [SMILES: COc1ccc(COC(=O)[C@H](Cc2ccccc2)NC(=O)[C@H](C)[C@@H](OC)[C@@H]2CCCN2)c(OC)c1]

as an HCl salt.

In a third aspect, a process for making

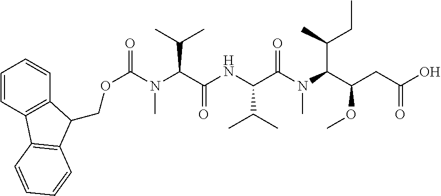 [SMILES: CC[C@H](C)[C@@H]([C@@H](CC(=O)O)OC)N(C)C(=O)[C@@H](NC(=O)[C@H](C(C)C)N(C)C(=O)OCC1c2ccccc2-c2ccccc21)C(C)C]

(5) is provided, the process comprising (i) reacting

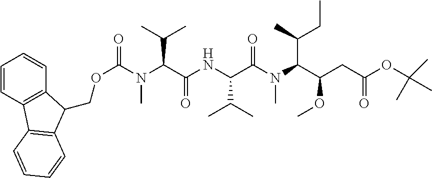 [SMILES: CC[C@H](C)[C@@H]([C@@H](CC(=O)OC(C)(C)C)OC)N(C)C(=O)[C@@H](NC(=O)[C@H](C(C)C)N(C)C(=O)OCC1c2ccccc2-c2ccccc21)C(C)C]

(6) in the presence of aqueous sulfuric acid, and (ii) isolating

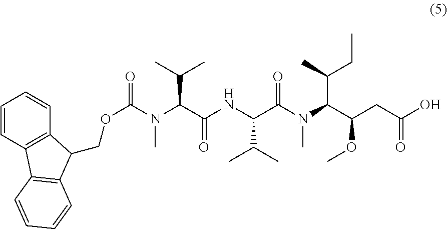 [SMILES: CC[C@H](C)[C@@H]([C@@H](CC(=O)O)OC)N(C)C(=O)[C@@H](NC(=O)[C@H](C(C)C)N(C)C(=O)OCC1c2ccccc2-c2ccccc21)C(C)C]

without a workup.

Other aspects and embodiments are provided herein.

### BRIEF DESCRIPTION OF THE FIGURES

FIG. 1 depicts the Blenrep® Structure.

FIG. 2 depicts the Blenrep® Mechanism of Action.

FIG. 3 depicts a first-generation process for making mcMMAF.

FIG. 4 depicts Process Mass Intensity (PMI) of each step per kg of packaged mcMMAF (first generation process).

FIG. 5 depicts Global Warming Potential (GWP) each step per kg of packaged mcMMAF (first generation process).

FIGS. 6A and 6B depict a second-generation process to mcMMAF.

FIG. 7 depicts a breakdown of PMI by class for the first-generation process to mcMMAF.

FIG. 8 depicts a breakdown of PMI by class for the second-generation process to mcMMAF.

FIGS. 9A and 9B depict a process metrics overview for first generation process.

### DETAILED DESCRIPTION OF THE INVENTION

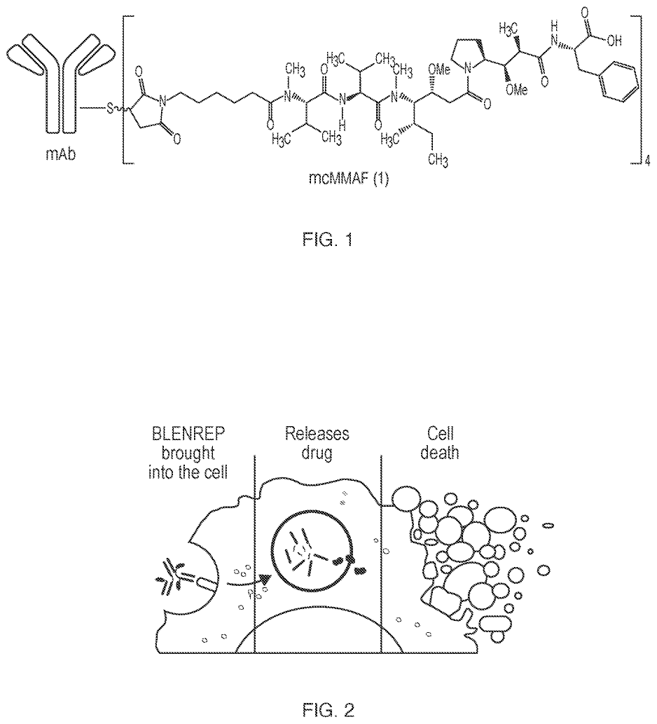

Blenrep® (FIG. 1) works by identifying and binding to BCMA (a cell-surface protein expressed on myeloma cells, late-stage B cells, and plasma cells (https://www.blenrephcp.com/clinical-data/how-blenrep-works/, herein incorporated by reference in its entirety). As Blenrep is brought into the cancerous myeloma cells, the cytotoxic payload (mcMMAF, 1) is released, which results in cell death (FIG. 2). The manufacturing route for mcMMAF (1) implemented in early clinical studies was also utilized for commercial production.

Herein, improved processes for mcMMAF are disclosed. Some embodiments relate to the development of a second generation (2G) manufacturing route for mcMMAF. The complexity of the mcMMAF peptide and the high potency categorization of the molecule requires special handling. The second-generation manufacturing route to mcMMAF reduces solvent consumption, reduces greenhouse gas emissions, reduces energy consumption, eliminates the need for single use silica gel chromatographic separations, and achieves overall PMI reduction. In some embodiments, the second-generation manufacturing route to mcMMAF is able to reduce solvent consumption by 16,160 kg/kg of mcMMAF, reduce greenhouse gas emissions by 71%, reduce energy consumption by 76%, eliminate the need for single use silica gel chromatographic separations and achieve an overall PMI reduction of 76%. In some embodiments, the 2G process has a smaller carbon footprint, uses less hazardous materials and solvents and is a more environmentally sustainable process.

In one aspect, provided herein is process for making maleimidocaproyl monomethyl auristatin F (1) comprising converting compound

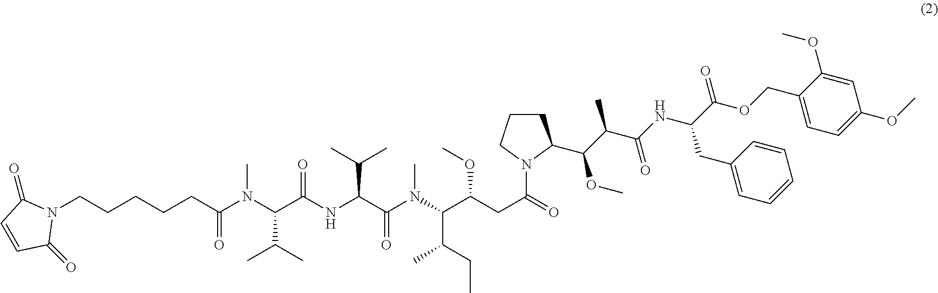 [SMILES: CC[C@H](C)[C@@H]([C@@H](CC(=O)N1CCC[C@H]1[C@H](OC)[C@@H](C)C(=O)N[C@@H](Cc1ccccc1)C(=O)OCc1ccc(OC)cc1OC)OC)N(C)C(=O)[C@@H](NC(=O)[C@H](C(C)C)N(C)C(=O)CCCCCN1C(=O)C=CC1=O)C(C)C]

to maleimidocaproyl monomethyl Auristatin F in the presence of toluene and 2-methyltetrahydrofuran, an aqueous workup, and chromatographic purification.

In some embodiments, the process further comprises a process for making

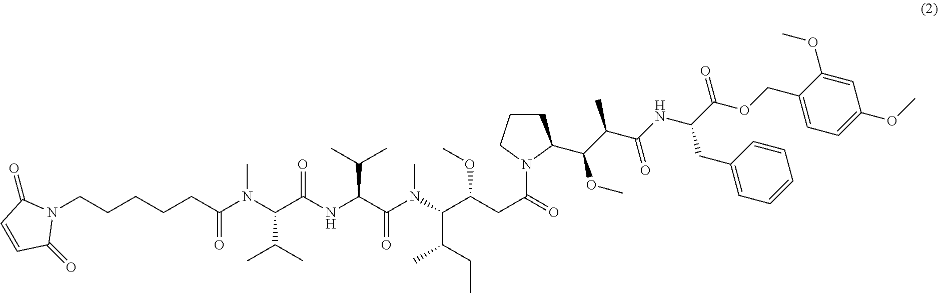 [SMILES: CC[C@H](C)[C@@H]([C@@H](CC(=O)N1CCC[C@H]1[C@H](OC)[C@@H](C)C(=O)N[C@@H](Cc1ccccc1)C(=O)OCc1ccc(OC)cc1OC)OC)N(C)C(=O)[C@@H](NC(=O)[C@H](C(C)C)N(C)C(=O)CCCCCN1C(=O)C=CC1=O)C(C)C]

wherein said process comprises reacting

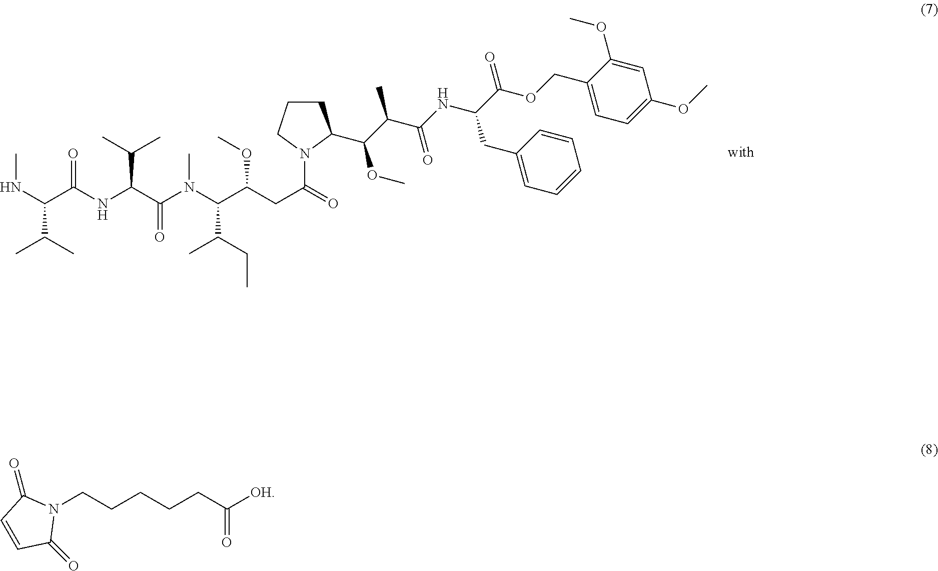 [SMILES: CCC(C)[C@@H]([C@@H](CC(=O)N1CCC[C@H]1[C@H](OC)[C@@H](C)C(=O)N[C@@H](Cc1ccccc1)C(=O)OCc1ccc(OC)cc1OC)OC)N(C)C(=O)[C@@H](NC(=O)[C@@H](NC)C(C)C)C(C)C ; O=C(O)CCCCCN1C(=O)C=CC1=O]

In some embodiments, the process further comprises a process for making

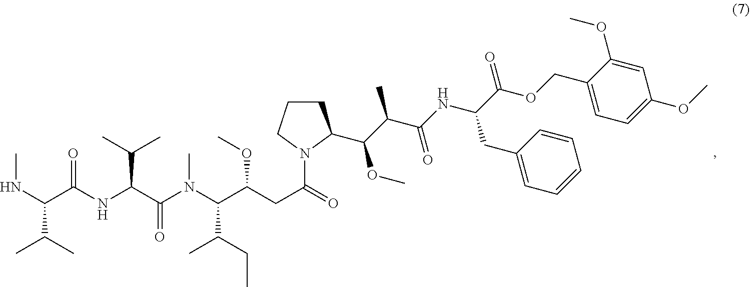 [SMILES: CCC(C)[C@@H]([C@@H](CC(=O)N1CCC[C@H]1[C@H](OC)[C@@H](C)C(=O)N[C@@H](Cc1ccccc1)C(=O)OCc1ccc(OC)cc1OC)OC)N(C)C(=O)[C@@H](NC(=O)[C@@H](NC)C(C)C)C(C)C]

wherein said process comprises reacting

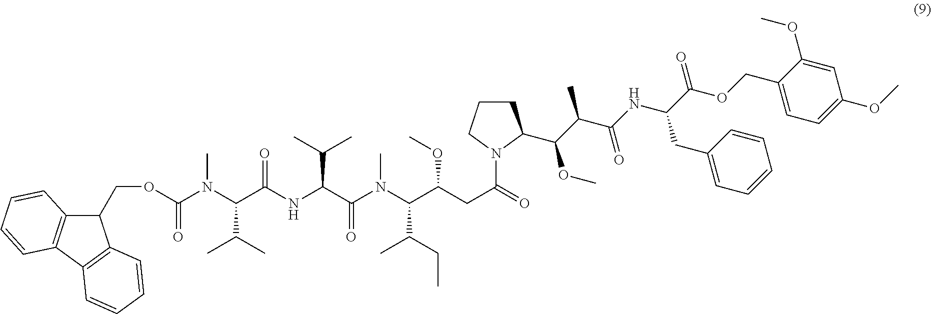 [SMILES: CCC(C)[C@@H]([C@@H](CC(=O)N1CCC[C@H]1[C@H](OC)[C@@H](C)C(=O)N[C@@H](Cc1ccccc1)C(=O)OCc1ccc(OC)cc1OC)OC)N(C)C(=O)[C@@H](NC(=O)[C@H](C(C)C)N(C)C(=O)OCC1c2ccccc2-c2ccccc21)C(C)C]

in the presence of a catalytic amount of DBU. In some embodiments,

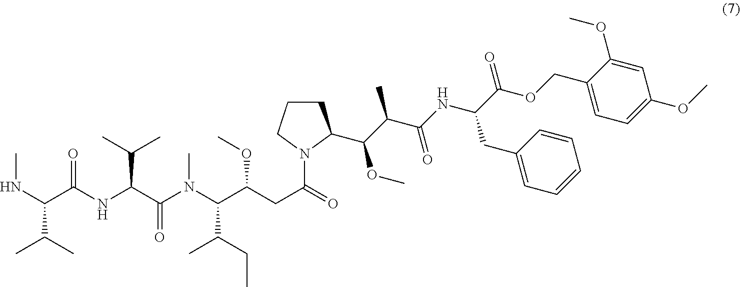 [SMILES: CCC(C)[C@@H]([C@@H](CC(=O)N1CCC[C@H]1[C@H](OC)[C@@H](C)C(=O)N[C@@H](Cc1ccccc1)C(=O)OCc1ccc(OC)cc1OC)OC)N(C)C(=O)[C@@H](NC(=O)[C@@H](NC)C(C)C)C(C)C]

is not isolated using column chromatography.

In some embodiments, the process further comprises a process for making

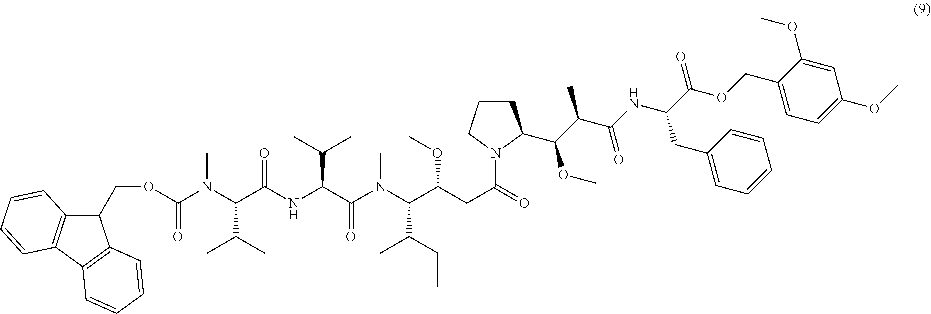 [SMILES: CCC(C)[C@@H]([C@@H](CC(=O)N1CCC[C@H]1[C@H](OC)[C@@H](C)C(=O)N[C@@H](Cc1ccccc1)C(=O)OCc1ccc(OC)cc1OC)OC)N(C)C(=O)[C@@H](NC(=O)[C@H](C(C)C)N(C)C(=O)OCC1c2ccccc2-c2ccccc21)C(C)C]

wherein said process comprises reacting

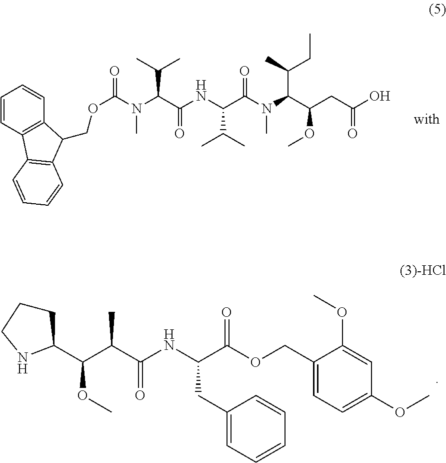 [SMILES: CC[C@H](C)[C@@H]([C@@H](CC(=O)O)OC)N(C)C(=O)[C@@H](NC(=O)[C@H](C(C)C)N(C)C(=O)OCC1c2ccccc2-c2ccccc21)C(C)C ; COc1ccc(COC(=O)[C@H](Cc2ccccc2)NC(=O)[C@H](C)[C@@H](OC)[C@@H]2CCCN2)c(OC)c1]

In some embodiments,

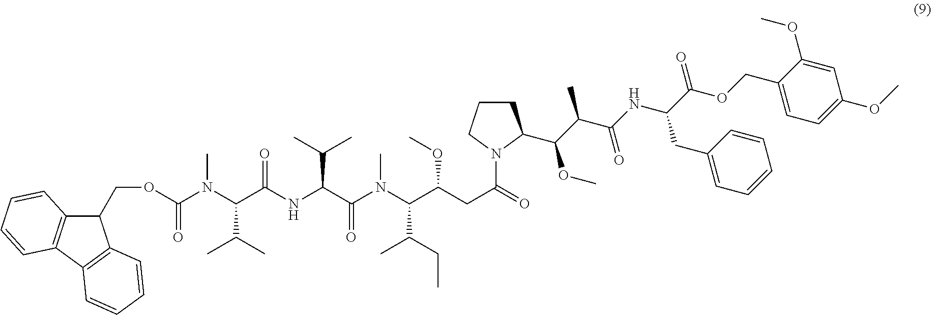 [SMILES: CCC(C)[C@@H]([C@@H](CC(=O)N1CCC[C@H]1[C@H](OC)[C@@H](C)C(=O)N[C@@H](Cc1ccccc1)C(=O)OCc1ccc(OC)cc1OC)OC)N(C)C(=O)[C@@H](NC(=O)[C@H](C(C)C)N(C)C(=O)OCC1c2ccccc2-c2ccccc21)C(C)C]

is not isolated using column chromatography.

In some embodiments, the process further comprises a process for making

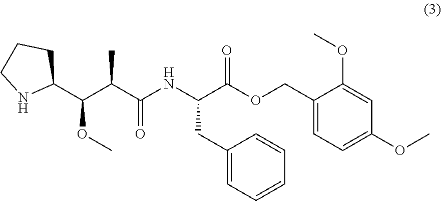 [SMILES: COc1ccc(COC(=O)[C@H](Cc2ccccc2)NC(=O)[C@H](C)[C@@H](OC)[C@@H]2CCCN2)c(OC)c1]

as an HCl salt, wherein said process comprises reacting

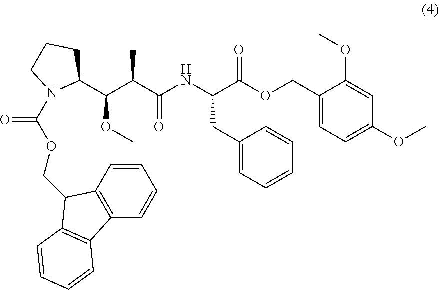 [SMILES: COc1ccc(COC(=O)[C@H](Cc2ccccc2)NC(=O)[C@H](C)[C@@H](OC)[C@@H]2CCCN2C(=O)OCC2c3ccccc3-c3ccccc32)c(OC)c1]

in the presence of a catalytic amount of DBU, and isolating

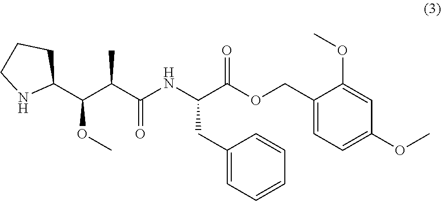 [SMILES: COc1ccc(COC(=O)[C@H](Cc2ccccc2)NC(=O)[C@H](C)[C@@H](OC)[C@@H]2CCCN2)c(OC)c1]

as a salt.

In some embodiments, the process further comprises a process for making

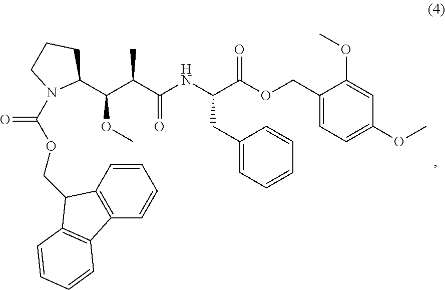 [SMILES: COc1ccc(COC(=O)[C@H](Cc2ccccc2)NC(=O)[C@H](C)[C@@H](OC)[C@@H]2CCCN2C(=O)OCC2c3ccccc3-c3ccccc32)c(OC)c1]

wherein said process comprises reacting the HCl salt of

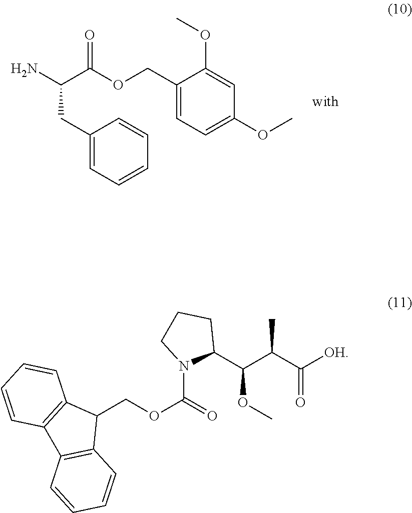 [SMILES: COc1ccc(COC(=O)[C@@H](N)Cc2ccccc2)c(OC)c1 ; CO[C@H]([C@@H](C)C(=O)O)[C@@H]1CCCN1C(=O)OCC1c2ccccc2-c2ccccc21]

In some embodiments,

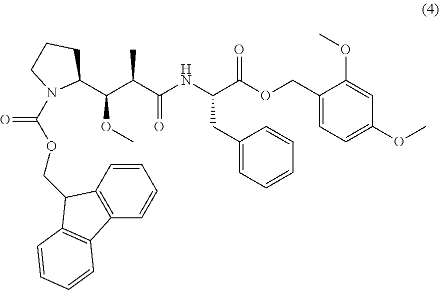 [SMILES: COc1ccc(COC(=O)[C@H](Cc2ccccc2)NC(=O)[C@H](C)[C@@H](OC)[C@@H]2CCCN2C(=O)OCC2c3ccccc3-c3ccccc32)c(OC)c1]

is not isolated using column chromatography.

In some embodiments, the process further comprises a process for making

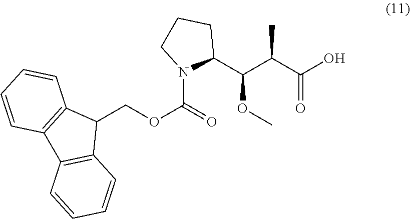 [SMILES: CO[C@H]([C@@H](C)C(=O)O)[C@@H]1CCCN1C(=O)OCC1c2ccccc2-c2ccccc21]

wherein said process comprises reacting the HCl salt of

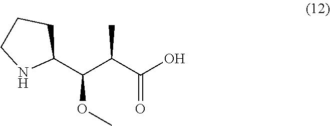

in the presence of water and dimethylformamide. In some embodiments,

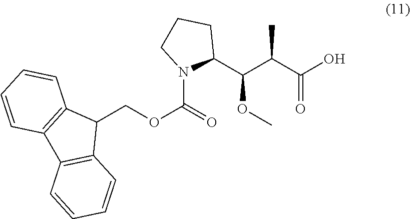 [SMILES: CO[C@H]([C@@H](C)C(=O)O)[C@@H]1CCCN1C(=O)OCC1c2ccccc2-c2ccccc21]

is not isolated using column chromatography.

In some embodiments, the process further comprises a process for making the HCl salt of

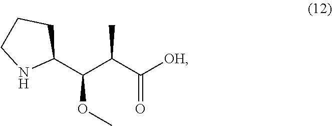

wherein said process comprises reacting

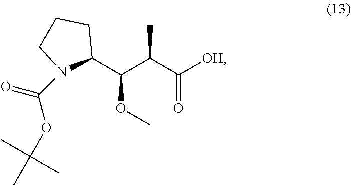 [SMILES: CO[C@H]([C@@H](C)C(=O)O)[C@@H]1CCCN1C(=O)OC(C)(C)C]

and isolating

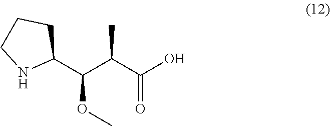

as an HCl salt.

In some embodiments, the process further comprises a process for making

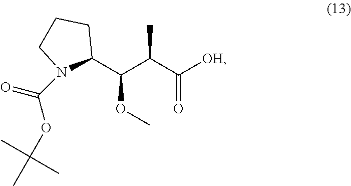 [SMILES: CO[C@H]([C@@H](C)C(=O)O)[C@@H]1CCCN1C(=O)OC(C)(C)C]

wherein said process comprises converting

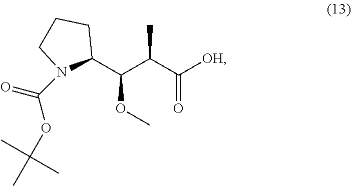 [SMILES: CO[C@H]([C@@H](C)C(=O)O)[C@@H]1CCCN1C(=O)OC(C)(C)C]

DCHA into

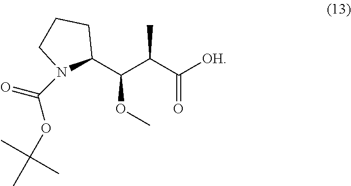 [SMILES: CO[C@H]([C@@H](C)C(=O)O)[C@@H]1CCCN1C(=O)OC(C)(C)C]

In some embodiments, the process further comprises a process for making

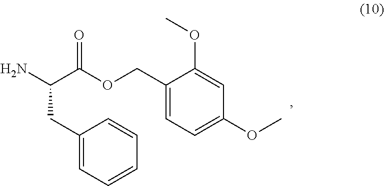 [SMILES: COc1ccc(COC(=O)[C@@H](N)Cc2ccccc2)c(OC)c1]

wherein said process comprises reacting

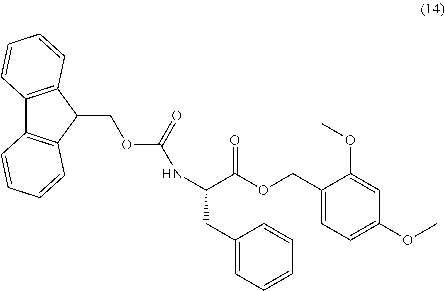 [SMILES: COc1ccc(COC(=O)[C@H](Cc2ccccc2)NC(=O)OCC2c3ccccc3-c3ccccc32)c(OC)c1]

in the presence of a catalytic amount of DBU, and isolating

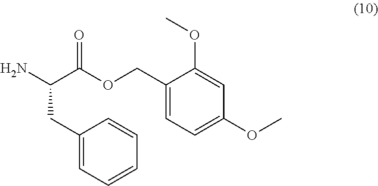 [SMILES: COc1ccc(COC(=O)[C@@H](N)Cc2ccccc2)c(OC)c1]

as an HCl salt.

In some embodiments, the process further comprises a process for making

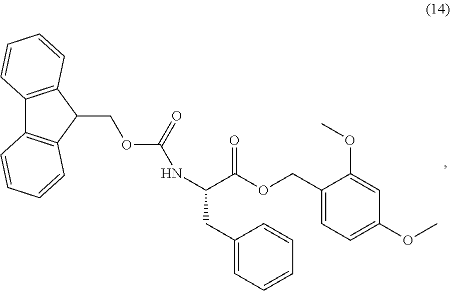 [SMILES: COc1ccc(COC(=O)[C@H](Cc2ccccc2)NC(=O)OCC2c3ccccc3-c3ccccc32)c(OC)c1]

wherein said process comprises converting

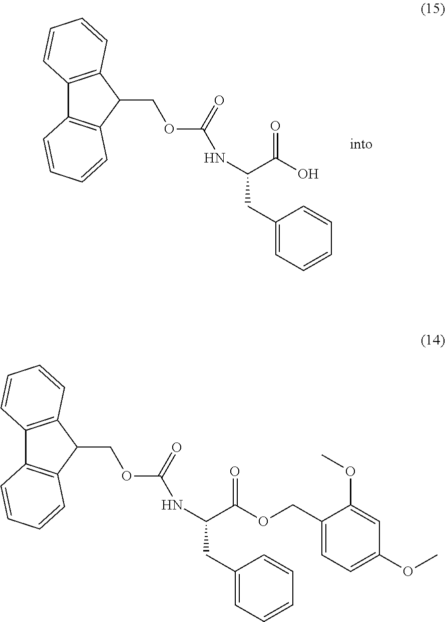 [SMILES: O=C(N[C@@H](Cc1ccccc1)C(=O)O)OCC1c2ccccc2-c2ccccc21 ; COc1ccc(COC(=O)[C@H](Cc2ccccc2)NC(=O)OCC2c3ccccc3-c3ccccc32)c(OC)c1]

in a solvent other than dichloromethane.

In some embodiments, the process further comprises a process for making

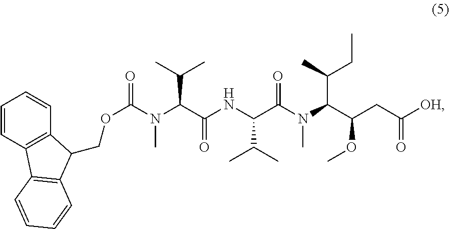 [SMILES: CC[C@H](C)[C@@H]([C@@H](CC(=O)O)OC)N(C)C(=O)[C@@H](NC(=O)[C@H](C(C)C)N(C)C(=O)OCC1c2ccccc2-c2ccccc21)C(C)C]

wherein said process comprises reacting

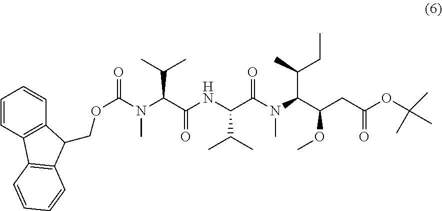 [SMILES: CC[C@H](C)[C@@H]([C@@H](CC(=O)OC(C)(C)C)OC)N(C)C(=O)[C@@H](NC(=O)[C@H](C(C)C)N(C)C(=O)OCC1c2ccccc2-c2ccccc21)C(C)C]

in the presence of aqueous sulfuric acid, and isolating

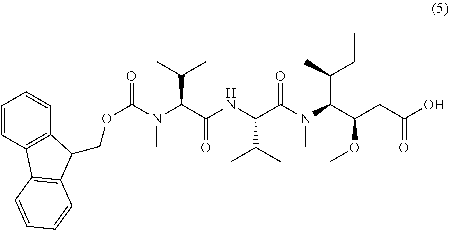 [SMILES: CC[C@H](C)[C@@H]([C@@H](CC(=O)O)OC)N(C)C(=O)[C@@H](NC(=O)[C@H](C(C)C)N(C)C(=O)OCC1c2ccccc2-c2ccccc21)C(C)C]

without a workup.

In some embodiments, the process further comprises a process for making

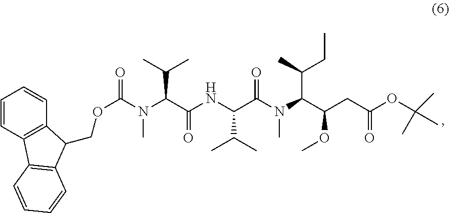 [SMILES: CC[C@H](C)[C@@H]([C@@H](CC(=O)OC(C)(C)C)OC)N(C)C(=O)[C@@H](NC(=O)[C@H](C(C)C)N(C)C(=O)OCC1c2ccccc2-c2ccccc21)C(C)C]

wherein said process comprises reacting

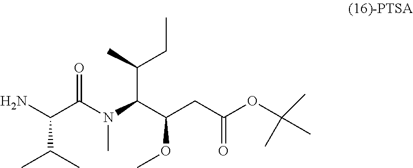 [SMILES: CC[C@H](C)[C@@H]([C@@H](CC(=O)OC(C)(C)C)OC)N(C)C(=O)[C@@H](N)C(C)C]

with Fmoc-NMeVal-OH.

In some embodiments, the process further comprises a process for making

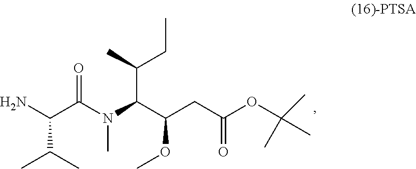 [SMILES: CC[C@H](C)[C@@H]([C@@H](CC(=O)OC(C)(C)C)OC)N(C)C(=O)[C@@H](N)C(C)C]

wherein said process comprises reacting

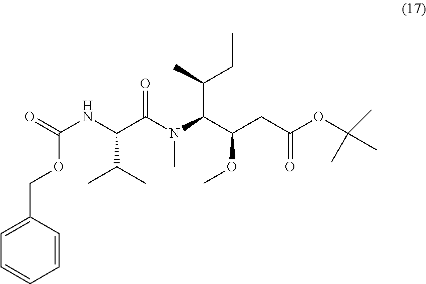 [SMILES: CC[C@H](C)[C@@H]([C@@H](CC(=O)OC(C)(C)C)OC)N(C)C(=O)[C@@H](NC(=O)OCc1ccccc1)C(C)C]

in the presence of 2-methyltetrahydrofuran.

In some embodiments, the process further comprises a process for making

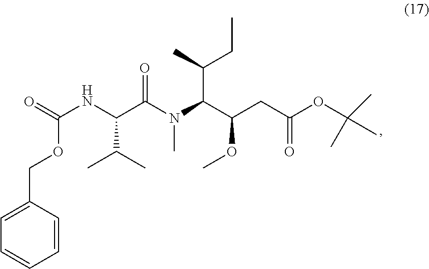 [SMILES: CC[C@H](C)[C@@H]([C@@H](CC(=O)OC(C)(C)C)OC)N(C)C(=O)[C@@H](NC(=O)OCc1ccccc1)C(C)C]

wherein said process comprises reacting

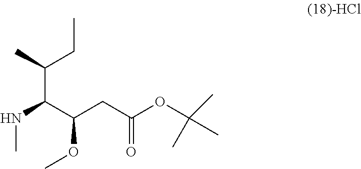 [SMILES: CC[C@H](C)[C@H](NC)[C@@H](CC(=O)OC(C)(C)C)OC]

with Z-Val-OH in the presence of Surfactant TPGS-750-M.

In some embodiments, the process supplies kilogram quantities of maleimidocaproyl monomethyl auristatin F.

In one aspect, provided herein is process for making maleimidocaproyl monomethyl auristatin F comprising converting compound

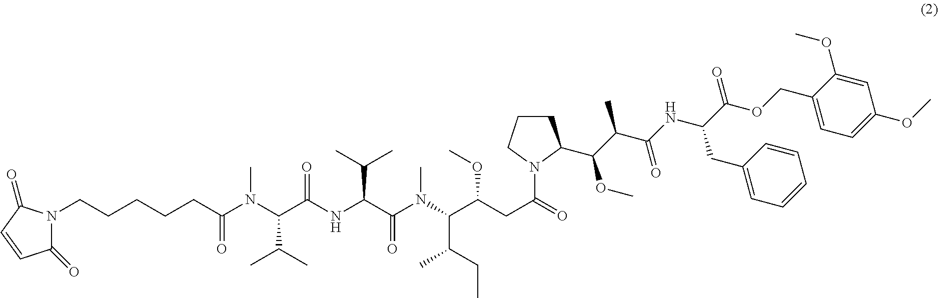 [SMILES: CC[C@H](C)[C@@H]([C@@H](CC(=O)N1CCC[C@H]1[C@H](OC)[C@@H](C)C(=O)N[C@@H](Cc1ccccc1)C(=O)OCc1ccc(OC)cc1OC)OC)N(C)C(=O)[C@@H](NC(=O)[C@H](C(C)C)N(C)C(=O)CCCCCN1C(=O)C=CC1=O)C(C)C]

to maleimidocaproyl monomethyl Auristatin F in the presence of toluene and 2-methyltetrahydrofuran, an aqueous workup, and chromatographic purification. In some embodiments of the process of converting (2) to maleimidocaproyl monomethyl Auristatin F,

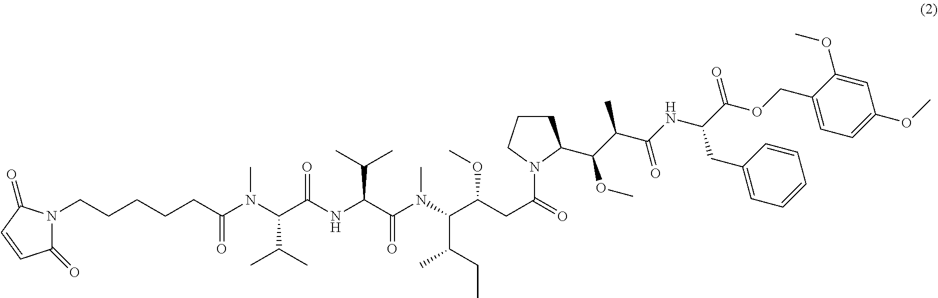 [SMILES: CC[C@H](C)[C@@H]([C@@H](CC(=O)N1CCC[C@H]1[C@H](OC)[C@@H](C)C(=O)N[C@@H](Cc1ccccc1)C(=O)OCc1ccc(OC)cc1OC)OC)N(C)C(=O)[C@@H](NC(=O)[C@H](C(C)C)N(C)C(=O)CCCCCN1C(=O)C=CC1=O)C(C)C]

is treated with an acid. In some embodiments of the process of converting (2) to maleimidocaproyl monomethyl Auristatin F, (2) is treated with trifluoroacetic acid.

In some embodiments of the process of converting (2) to maleimidocaproyl monomethyl Auristatin F, (2) is reacted with trifluoroacetic acid at about −5° C. to about 5° C.

In some embodiments of the process of converting (2) to maleimidocaproyl monomethyl Auristatin F, (2) is reacted with trifluoroacetic acid for about 4 hours to about 6 hours. In some embodiments of the process of converting (2) to maleimidocaproyl monomethyl Auristatin F, (2) is reacted with trifluoroacetic acid for about 3 hours to about 7 hours.

In some embodiments of the process of converting (2) to maleimidocaproyl monomethyl Auristatin F, maleimidocaproyl monomethyl Auristatin F is purified by column chromatography using dichloromethane as the loading solvent and eluting with 2-propanol in dichloromethane.

In some embodiments, the process further comprises a process for making

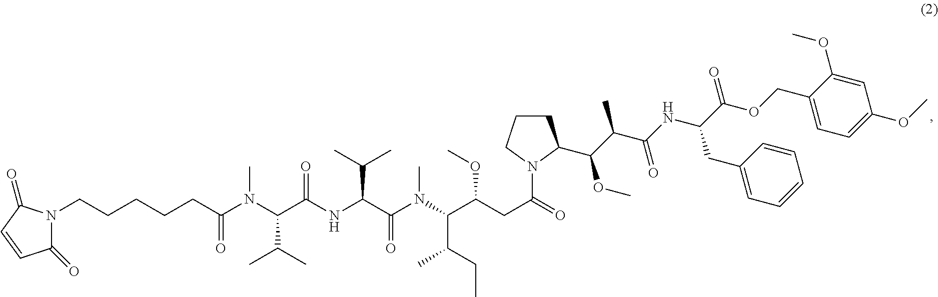 [SMILES: CC[C@H](C)[C@@H]([C@@H](CC(=O)N1CCC[C@H]1[C@H](OC)[C@@H](C)C(=O)N[C@@H](Cc1ccccc1)C(=O)OCc1ccc(OC)cc1OC)OC)N(C)C(=O)[C@@H](NC(=O)[C@H](C(C)C)N(C)C(=O)CCCCCN1C(=O)C=CC1=O)C(C)C]

wherein said process comprises reacting

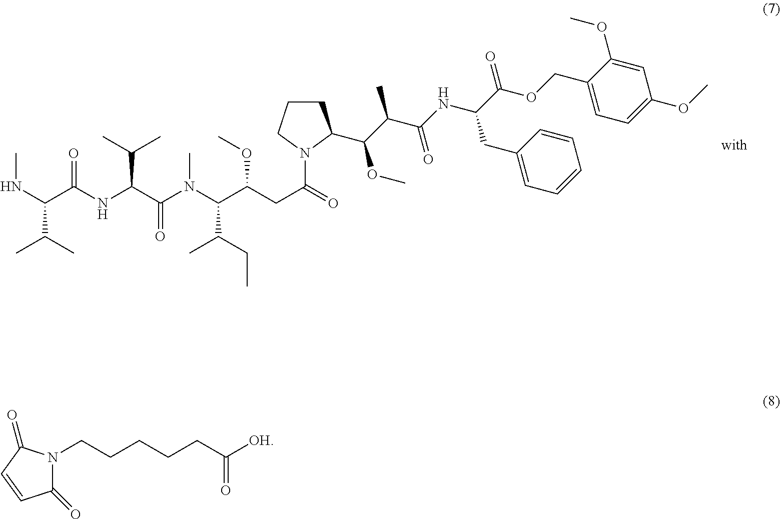 [SMILES: CCC(C)[C@@H]([C@@H](CC(=O)N1CCC[C@H]1[C@H](OC)[C@@H](C)C(=O)N[C@@H](Cc1ccccc1)C(=O)OCc1ccc(OC)cc1OC)OC)N(C)C(=O)[C@@H](NC(=O)[C@@H](NC)C(C)C)C(C)C ; O=C(O)CCCCCN1C(=O)C=CC1=O]

In some embodiments of the process of making (2) comprising reacting (7) with (8), (2) is not isolated using column chromatography. In some embodiments of the process of making (2), reacting (7) with (8) occurs in the presence of a tertiary amine base. In some embodiments of the process of making (2), reacting (7) with (8) occurs in the presence of N,N-diisopropylethylamine. In some embodiments of making (2), reacting (7) with (8) occurs in the presence of a coupling reagent. In some embodiments of making (2), reacting (7) with (8) occurs in the presence of 2-chloro-1-methylpyridinium iodide.

In some embodiments of the process of making (2), reacting (7) with (8) occurs in the presence of N,N-diisopropylethylamine and 2-chloro-1-methylpyridinium iodide at about 20° C. to about 30° C.

In some embodiments of the process of making (2), reacting (7) with (8) occurs in the presence of N,N-diisopropylethylamine and 2-chloro-1-methylpyridinium iodide for not less than about 5 hours.

In some embodiments of the process of making (2), reacting (7) with (8) occurs in the presence of N,N-diisopropylethylamine and 2-chloro-1-methylpyridinium iodide, wherein the reaction mixture is diluted with toluene after about 5 hours. In some embodiments of the process of making (2), reacting (7) with (8) occurs in the presence of N,N-diisopropylethylamine and 2-chloro-1-methylpyridinium iodide, wherein the mixture is diluted with toluene after about 5 hours, washed with aqueous sulfuric acid and water, and distilled exchanging acetonitrile for toluene.

In some embodiments, the process further comprises a process for making

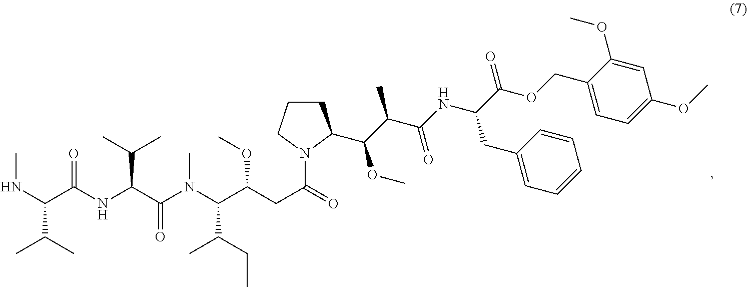 [SMILES: CCC(C)[C@@H]([C@@H](CC(=O)N1CCC[C@H]1[C@H](OC)[C@@H](C)C(=O)N[C@@H](Cc1ccccc1)C(=O)OCc1ccc(OC)cc1OC)OC)N(C)C(=O)[C@@H](NC(=O)[C@@H](NC)C(C)C)C(C)C]

wherein said process comprises reacting

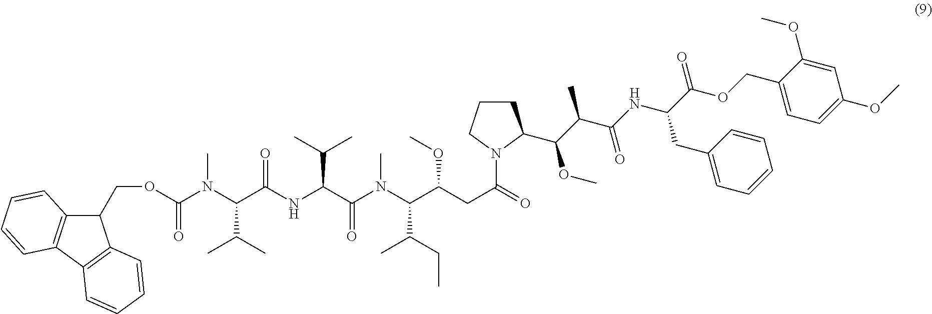 [SMILES: CCC(C)[C@@H]([C@@H](CC(=O)N1CCC[C@H]1[C@H](OC)[C@@H](C)C(=O)N[C@@H](Cc1ccccc1)C(=O)OCc1ccc(OC)cc1OC)OC)N(C)C(=O)[C@@H](NC(=O)[C@H](C(C)C)N(C)C(=O)OCC1c2ccccc2-c2ccccc21)C(C)C]

in the presence of a catalytic amount of DBU. In some embodiments of the process of making (7) comprising reacting (9) with a catalytic amount of DBU, (7) is not isolated using column chromatography.

In some embodiments of the process of making (7), (9) is reacted with a catalytic amount of DBU at about 20° C. to about 30° C.

In some embodiments of the process of making (7) comprising reacting (9) with a catalytic amount of DBU, the mixture is washed with heptane.

In some embodiments, the process further comprises a process for making

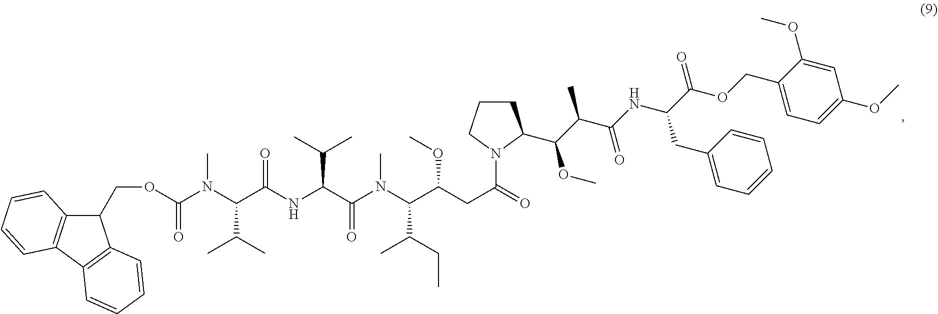 [SMILES: CCC(C)[C@@H]([C@@H](CC(=O)N1CCC[C@H]1[C@H](OC)[C@@H](C)C(=O)N[C@@H](Cc1ccccc1)C(=O)OCc1ccc(OC)cc1OC)OC)N(C)C(=O)[C@@H](NC(=O)[C@H](C(C)C)N(C)C(=O)OCC1c2ccccc2-c2ccccc21)C(C)C]

wherein said process comprises reacting

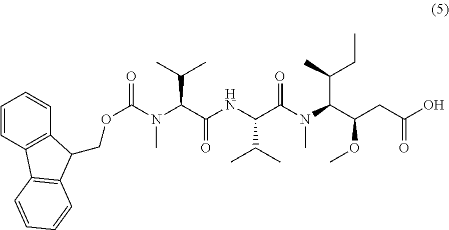 [SMILES: CC[C@H](C)[C@@H]([C@@H](CC(=O)O)OC)N(C)C(=O)[C@@H](NC(=O)[C@H](C(C)C)N(C)C(=O)OCC1c2ccccc2-c2ccccc21)C(C)C]

with the HCl salt of

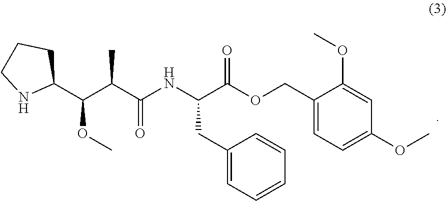 [SMILES: COc1ccc(COC(=O)[C@H](Cc2ccccc2)NC(=O)[C@H](C)[C@@H](OC)[C@@H]2CCCN2)c(OC)c1]

In some embodiments of the process of making (9) comprising reacting (5) with the HCl salt of (3), (9) is not isolated using column chromatography. In some embodiments of the process of making (9), reacting (5) with (3) occurs in the presence of a tertiary amine base. In some embodiments of the process of making (9), reacting (5) with (3) occurs in the presence of N,N-diisopropylethylamine. In some embodiments of the process of making (9), reacting (5) with (3) occurs in the presence of a coupling reagent. In some embodiments of the process of making (9), reacting (5) with (3) occurs in the presence of O-(benzotriazol-1-yl)-N,N,N′,N′-tetramethyluronium tetrafluoroborate.

In some embodiments of the process of making (9), reacting (5) with (3) occurs in the presence of N,N-diisopropylethylamine and O-(benzotriazol-1-yl)-N,N,N′,N′-tetramethyluronium tetrafluoroborate at about 20° C. to about 30° C.

In some embodiments of the process of making (9), reacting (5) with (3) occurs in the presence of N,N-diisopropylethylamine and O-(benzotriazol-1-yl)-N,N,N′,N′-tetramethyluronium tetrafluoroborate for not less than about 19 hours.

In some embodiments of the process of making (9), reacting (5) with (3) occurs in the presence of N,N-diisopropylethylamine and O-(benzotriazol-1-yl)-N,N,N′,N′-tetramethyluronium tetrafluoroborate, wherein after not less than about 19 hours, the mixture is diluted with heptane, washed with aqueous sulfuric acid, water, and aqueous sodium bicarbonate, and distilled exchanging ethyl acetate with acetonitrile.

In some embodiments, the process further comprises a process for making the HCl salt of

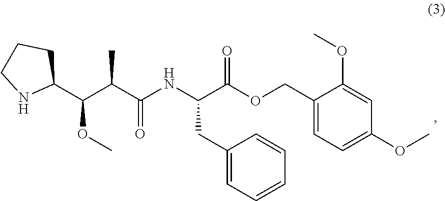 [SMILES: COc1ccc(COC(=O)[C@H](Cc2ccccc2)NC(=O)[C@H](C)[C@@H](OC)[C@@H]2CCCN2)c(OC)c1]

wherein said process comprises reacting

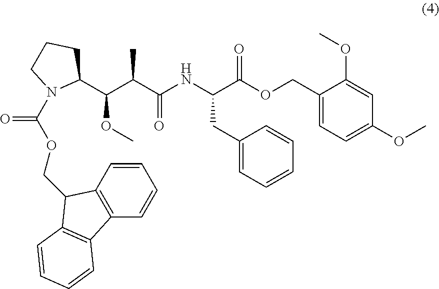 [SMILES: COc1ccc(COC(=O)[C@H](Cc2ccccc2)NC(=O)[C@H](C)[C@@H](OC)[C@@H]2CCCN2C(=O)OCC2c3ccccc3-c3ccccc32)c(OC)c1]

in the presence of a catalytic amount of DBU, and isolating

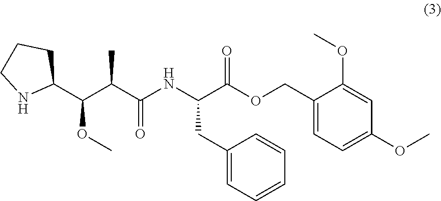 [SMILES: COc1ccc(COC(=O)[C@H](Cc2ccccc2)NC(=O)[C@H](C)[C@@H](OC)[C@@H]2CCCN2)c(OC)c1]

as a salt. In some embodiments of the process of making HCl salt of (3), (4) is treated with a thiol. In some embodiments of the process of making HCl salt of (3), (4) is treated with 1-octanethiol. In some embodiments of the process of making HCl salt of (3), 1,4-dioxane and (3)-HCl seed are added prior to isolating (3) as the HCl salt.

In some embodiments of the process of making HCl salt of (3), (4) is treated with 1-octanethiol at about 3° C. to about 9° C.

In some embodiments of the process of making HCl salt of (3), (4) is treated with 1-octanethiol for about 6 to about 8 hours.

In some embodiments of the process of making HCl salt of (3), (4) is treated with 1-octanethiol and DBU, the reaction mixture is washed with water after about 6-8 hours at about 3° C. to about 9° C. and diluted with ethyl acetate.

In some embodiments of the process of making HCl salt of (3), 1,4-dioxane and (3)-HCl seed are added at about −5° C. to about 5° C. prior to isolating (3) as the HCl salt.

In some embodiments of the process of making HCl salt of (3), (3)-HCl is crystallized, wherein additional hydrochloric acid in 1,4-dioxane is added over not less than about 30 minutes.

In some embodiments, the process further comprises a process for making

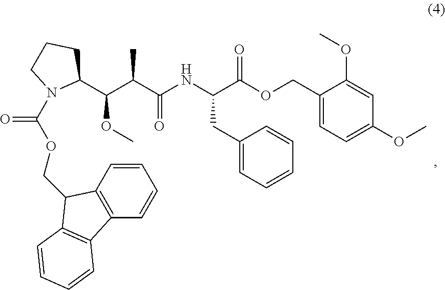 [SMILES: COc1ccc(COC(=O)[C@H](Cc2ccccc2)NC(=O)[C@H](C)[C@@H](OC)[C@@H]2CCCN2C(=O)OCC2c3ccccc3-c3ccccc32)c(OC)c1]

wherein said process comprises reacting the HCl salt of

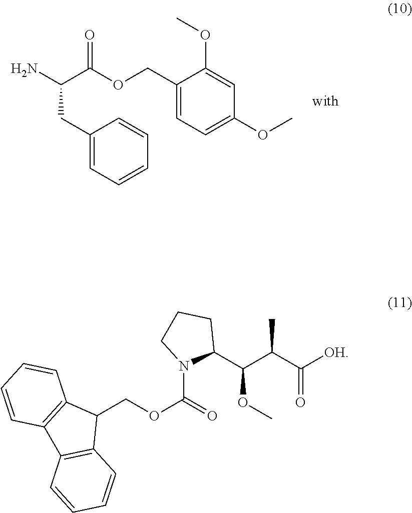 [SMILES: COc1ccc(COC(=O)[C@@H](N)Cc2ccccc2)c(OC)c1 ; CO[C@H]([C@@H](C)C(=O)O)[C@@H]1CCCN1C(=O)OCC1c2ccccc2-c2ccccc21]

In some embodiments of the process of making (4), (4) is not isolated using column chromatography. In some embodiments of the process of making (4), reacting (10) with (11) occurs in the presence of a coupling reagent. In some embodiments of the process of making (4), reacting (10) with (11) occurs in the presence of O-(benzotriazol-1-yl)-N,N,N′,N′-tetramethyluronium tetrafluoroborate. In some embodiments of the process of making (4), reacting (10) with (11) occurs in the presence of a tertiary amine base. In some embodiments of the process of making (4), reacting (10) with (11) occurs in the presence of N,N-diisopropylethylamine.

In some embodiments of the process of making (4), reacting (10) with (11) occurs in the presence of O-(benzotriazol-1-yl)-N,N,N′,N′-tetramethyluronium tetrafluoroborate and N,N-diisopropylethylamine at about 10° C. to about 20° C.

In some embodiments of the process of making (4), reacting (10) with (11) occurs in the presence of O-(benzotriazol-1-yl)-N,N,N′,N′-tetramethyluronium tetrafluoroborate and N,N-diisopropylethylamine for not less than about 6 hours.

In some embodiments of the process of making (4), reacting (10) with (11) occurs in the presence of O-(benzotriazol-1-yl)-N,N,N′,N′-tetramethyluronium tetrafluoroborate and N,N-diisopropylethylamine, wherein the reaction mixture is washed with water, aqueous hydrochloric acid, and aqueous potassium phosphate dibasic then distilled. In some embodiments of the process of making (4), reacting (10) with (11) occurs in the presence of O-(benzotriazol-1-yl)-N,N,N′,N′-tetramethyluronium tetrafluoroborate and N,N-diisopropylethylamine, wherein additional ethyl acetate is added to the reaction mixture.

In some embodiments, the process further comprises a process for making

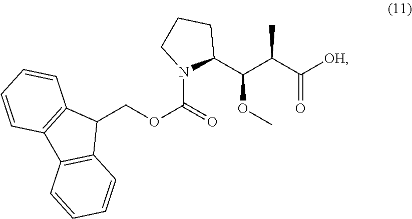 [SMILES: CO[C@H]([C@@H](C)C(=O)O)[C@@H]1CCCN1C(=O)OCC1c2ccccc2-c2ccccc21]

wherein said process comprises reacting the HCl salt of

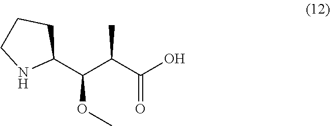

in the presence of water and dimethylformamide. In some embodiments of the process of making (11),

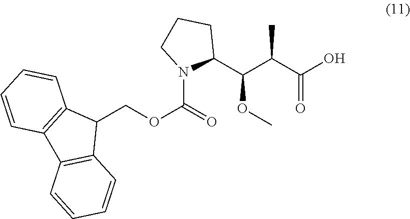 [SMILES: CO[C@H]([C@@H](C)C(=O)O)[C@@H]1CCCN1C(=O)OCC1c2ccccc2-c2ccccc21]

is not isolated using column chromatography. In some embodiments of the process of making (11), reacting (12)-HCl occurs in the presence of water, dimethylformamide, and a reagent for the selective formation of a carbamate. In some embodiments of the process of making (11), reacting (12) occurs in the presence of water, dimethylformamide, and N-(9H-fluorenylmethoxycarbonyloxy)succinimide.

In some embodiments of the process of making (11), reacting (12)-HCl with Fmoc-OSu and sodium bicarbonate occurs in DMF-water at about 30° C. to about 40° C.

In some embodiments of the process of making (11), reacting (12)-HCl with Fmoc-OSu and sodium bicarbonate occurs in DMF-water for not less than about 9 hours.

In some embodiments of the process of making (11), reacting (12)-HCl with Fmoc-OSu and sodium bicarbonate occurs in DMF-water, wherein after not less than about 9 hours heptane is added to the mixture to separate the phases, ethyl acetate, water, and phosphoric acid are charged to the aqueous layer, the phases are separated, and the organic layer is washed with water.

In some embodiments, the process further comprises a process for making the HCl salt of

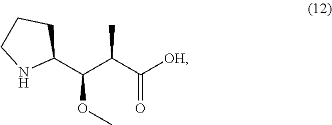

wherein said process comprises contacting the DCHA salt of

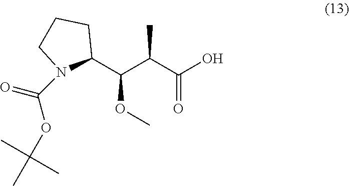 [SMILES: CO[C@H]([C@@H](C)C(=O)O)[C@@H]1CCCN1C(=O)OC(C)(C)C]

with an acid, and isolating

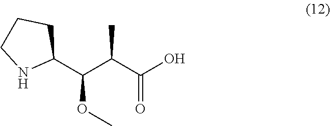

as an HCl salt. In some embodiments of the process of making the HCl salt of (12),

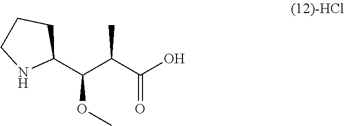

is isolated by filtration. In some embodiments of the process of making the HCl salt of (12), heptane and

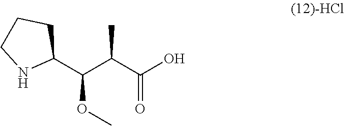

seed are added prior to isolating (12) as the HCl salt.

In some embodiments of the process of making of HCl salt of (12), (13) is contacted with HCl in 1,4-dioxane at about 15° C. to about 25° C.

In some embodiments of the process of making of HCl salt of (12), (13) is contacted with HCl in 1,4-dioxane for about 8 to about 16 hours.

In some embodiments of the process of making of HCl salt of (12), (12)-HCl seed and heptane are added to the reaction mixture and the resulting mixture is held for not less than about 2.5 hours prior to filtration.

In some embodiments, the process further comprises a process for making

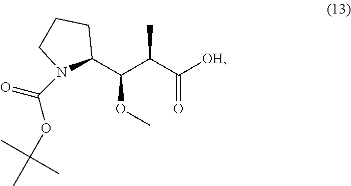 [SMILES: CO[C@H]([C@@H](C)C(=O)O)[C@@H]1CCCN1C(=O)OC(C)(C)C]

wherein said process comprises converting

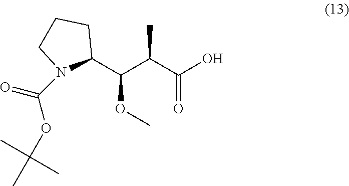 [SMILES: CO[C@H]([C@@H](C)C(=O)O)[C@@H]1CCCN1C(=O)OC(C)(C)C]

DCHA into

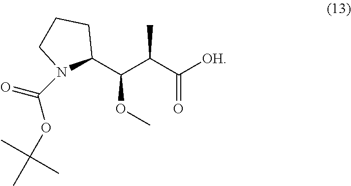 [SMILES: CO[C@H]([C@@H](C)C(=O)O)[C@@H]1CCCN1C(=O)OC(C)(C)C]

In some embodiments of the process of making (13), converting (13) DCHA into (13) occurs in the presence of an acid. In some embodiments of the process of making (13), converting (13) DCHA into (13) occurs in the presence of sulfuric acid. In some embodiments of the process of making (13), (13) DCHA is washed with aqueous sulfuric acid then water and replaced with 1,4-dioxane by distillation.

In some embodiments, the process further comprises a process for making

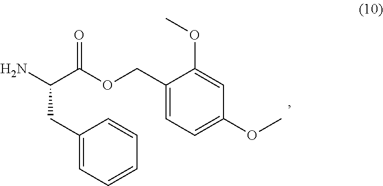 [SMILES: COc1ccc(COC(=O)[C@@H](N)Cc2ccccc2)c(OC)c1]

wherein said process comprises reacting

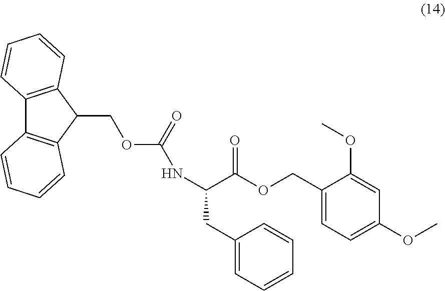 [SMILES: COc1ccc(COC(=O)[C@H](Cc2ccccc2)NC(=O)OCC2c3ccccc3-c3ccccc32)c(OC)c1]

in the presence of a catalytic amount of DBU, and isolating

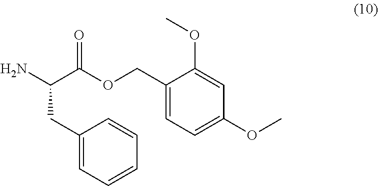 [SMILES: COc1ccc(COC(=O)[C@@H](N)Cc2ccccc2)c(OC)c1]

as an HCl salt. In some embodiments of the process of making (10) in the presence of a catalytic amount of DBU, (14) is treated with a thiol. In some embodiments of the process of making (10) in the presence of a catalytic amount of DBU, (14) is treated with 1-octanethiol. In some embodiments of the process of making (10) in the presence of a catalytic amount of DBU, (10)-HCl is crystallized. In some embodiments of the process of making (10) in the presence of a catalytic amount of DBU, 1,4-dioxane is added prior to isolating (10) as the HCl salt.

In some embodiments of the process of making (10) in the presence of a catalytic amount of DBU, (14) is reacted with 1-octanethiol at about 25° C. to about 36° C.

In some embodiments of the process of making (10) in the presence of a catalytic amount of DBU, (14) is reacted with 1-octanethiol for about 8 to about 24 hours.

In some embodiments of the process of making (10) in the presence of a catalytic amount of DBU, (14) is reacted with 1-octanethiol, washed with water and diluted with ethyl acetate.

In some embodiments, the process further comprises a process for making

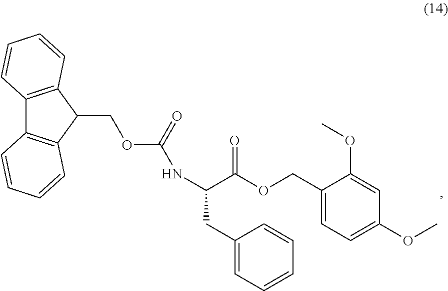 [SMILES: COc1ccc(COC(=O)[C@H](Cc2ccccc2)NC(=O)OCC2c3ccccc3-c3ccccc32)c(OC)c1]

wherein said process comprises reacting

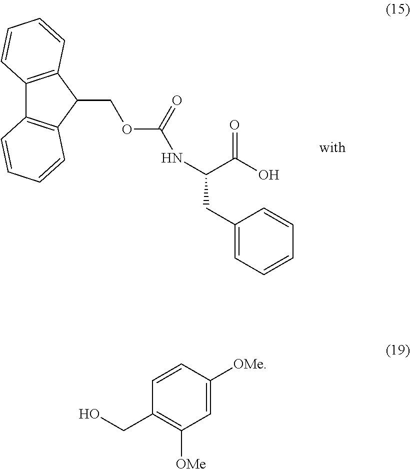 [SMILES: O=C(N[C@@H](Cc1ccccc1)C(=O)O)OCC1c2ccccc2-c2ccccc21 ; COc1ccc(CO)c(OC)c1]

In some embodiments of the process of making (14), reacting (15) with (19) occurs in a solvent other than dichloromethane. In some embodiments of the process of making (14), (14) is not isolated using column chromatography. In some embodiments of the process of making (14), converting (15) into (14) occurs in the presence of a catalyst. In some embodiments of the process of making (14), the catalyst present in converting (15) into (14) is 4-dimethylaminopyridine. In some embodiments of the process of making (14), converting (15) into (14) occurs in the presence of a coupling agent. In some embodiments of the process of making (14), the coupling agent present in converting (15) into (14) is N,N′-diisopropylcarbodiimide. In some embodiments of the process of making (14), converting (15) into (14) occurs in an ester solvent. In some embodiments of the process of making (14), converting (15) into (14) occurs in ethyl acetate.

In some embodiments of the process of making (14), reacting (15) with (19) occurs in the presence of catalyst 4-dimethylaminopyridine and coupling agent N,N′-diisopropylcarbodiimide in ethyl acetate at about −6° C. to about 6° C. for not less than about 20 hours. In some embodiments of the process of making (14), reacting (15) with (19) occurs in the presence of catalyst 4-dimethylaminopyridine and coupling agent N,N′-diisopropylcarbodiimide in ethyl acetate at about −6° C. to about 6° C.

In some embodiments of the process of making (14), reacting (15) with (19) occurs in the presence of catalyst 4-dimethylaminopyridine and coupling agent N,N′-diisopropylcarbodiimide in ethyl acetate for not less than about 20 hours.

In some embodiments of the process of making (14), reacting (15) with (19) occurs in the presence of catalyst 4-dimethylaminopyridine and coupling agent N,N′-diisopropylcarbodiimide in ethyl acetate, the reaction mixture is washed with sodium chloride in aqueous phosphoric acid followed by aqueous potassium phosphate dibasic, and distilled from ethyl acetate to give (14).

In some embodiments, the process further comprises a process for making

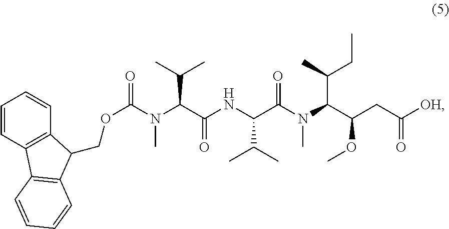 [SMILES: CC[C@H](C)[C@@H]([C@@H](CC(=O)O)OC)N(C)C(=O)[C@@H](NC(=O)[C@H](C(C)C)N(C)C(=O)OCC1c2ccccc2-c2ccccc21)C(C)C]

wherein said process comprises reacting

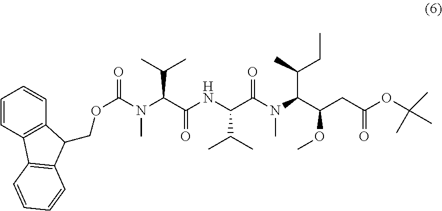 [SMILES: CC[C@H](C)[C@@H]([C@@H](CC(=O)OC(C)(C)C)OC)N(C)C(=O)[C@@H](NC(=O)[C@H](C(C)C)N(C)C(=O)OCC1c2ccccc2-c2ccccc21)C(C)C]

in the presence of aqueous sulfuric acid, and isolating

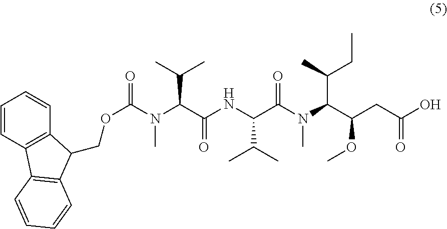 [SMILES: CC[C@H](C)[C@@H]([C@@H](CC(=O)O)OC)N(C)C(=O)[C@@H](NC(=O)[C@H](C(C)C)N(C)C(=O)OCC1c2ccccc2-c2ccccc21)C(C)C]

without a workup. In some embodiments of the process of making (5),

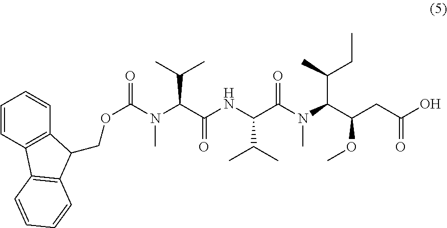 [SMILES: CC[C@H](C)[C@@H]([C@@H](CC(=O)O)OC)N(C)C(=O)[C@@H](NC(=O)[C@H](C(C)C)N(C)C(=O)OCC1c2ccccc2-c2ccccc21)C(C)C]

seed and water are added to the reaction mixture containing (6).

In some embodiments of the process of making (5), (6) is diluted with acetonitrile and water and reacted with aqueous sulfuric acid at about 5° C. to about 15° C. for not less than about 12 hours. In some embodiments of the process of making (5), (6) is diluted with acetonitrile and water and reacted with aqueous sulfuric acid at about 5° C. to about 15° C.

In some embodiments of the process of making (5), (6) is diluted with acetonitrile and water and reacted with aqueous sulfuric acid for not less than about 12 hours.

In some embodiments of the process of making (5), (5) seed and water are added to the mixture and cooled to about −5° C. to about 5° C. to isolate (5).

In some embodiments, the process further comprises a process for making

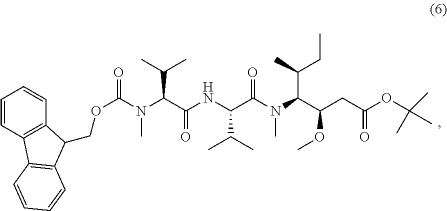 [SMILES: CC[C@H](C)[C@@H]([C@@H](CC(=O)OC(C)(C)C)OC)N(C)C(=O)[C@@H](NC(=O)[C@H](C(C)C)N(C)C(=O)OCC1c2ccccc2-c2ccccc21)C(C)C]

wherein said process comprises reacting

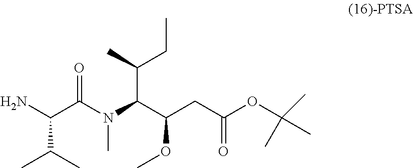 [SMILES: CC[C@H](C)[C@@H]([C@@H](CC(=O)OC(C)(C)C)OC)N(C)C(=O)[C@@H](N)C(C)C]

with Fmoc-NmeVal-OH. In some embodiments of the process of making (6),

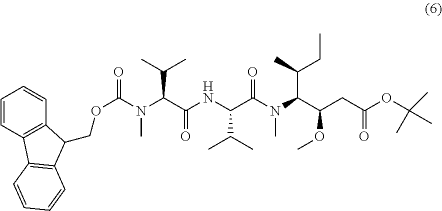 [SMILES: CC[C@H](C)[C@@H]([C@@H](CC(=O)OC(C)(C)C)OC)N(C)C(=O)[C@@H](NC(=O)[C@H](C(C)C)N(C)C(=O)OCC1c2ccccc2-c2ccccc21)C(C)C]

is not isolated using column chromatography. In some embodiments, reacting (16) with Fmoc-NmeVal-OH to form (6) occurs in the presence of a tertiary amine base. In some embodiments of the process of making (6), reacting (16) with Fmoc-NmeVal-OH to form (6) occurs in the presence of N,N-diisopropylethylamine. In some embodiments of the process of making (6), reacting (16) with Fmoc-NmeVal-OH to form (6) occurs in the presence of a coupling reagent. In some embodiments of the process of making (6), reacting (16) with Fmoc-NmeVal-OH to form (6) occurs in the presence of O-(benzotriazol-1-yl)-N,N,N′,N′-tetramethyluronium tetrafluoroborate.

In some embodiments of the process of making (6), (16)-PTSA is reacted with Fmoc-NMeVal-OH in the presence of O-(benzotriazol-1-yl)-N,N,N′,N′-tetramethyluronium in ethyl acetate at about 15° C. to about 25° C. for not less than about 6 hours. In some embodiments of the process of making (6), (16)-PTSA is reacted with Fmoc-NMeVal-OH in the presence of O-(benzotriazol-1-yl)-N,N,N′,N′-tetramethyluronium in ethyl acetate at about 15° C. to about 25° C.

In some embodiments of the process of making (6), (16)-PTSA is reacted with Fmoc-NMeVal-OH in the presence of O-(benzotriazol-1-yl)-N,N,N′,N′-tetramethyluronium in ethyl acetate for not less than about 6 hours.

In some embodiments of the process of making (6), (16)-PTSA is reacted with Fmoc-NMeVal-OH in the presence of O-(benzotriazol-1-yl)-N,N,N′,N′-tetramethyluronium in ethyl acetate, the reaction mixture is washed with water and distilled, replacing ethyl acetate with acetonitrile to give (6).

In some embodiments, the process further comprises a process for making

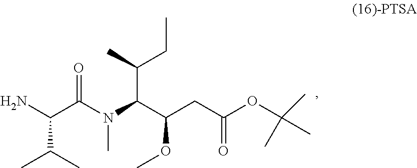 [SMILES: CC[C@H](C)[C@@H]([C@@H](CC(=O)OC(C)(C)C)OC)N(C)C(=O)[C@@H](N)C(C)C]

wherein said process comprises (a) hydrogenating

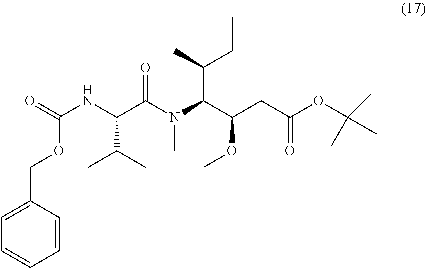 [SMILES: CC[C@H](C)[C@@H]([C@@H](CC(=O)OC(C)(C)C)OC)N(C)C(=O)[C@@H](NC(=O)OCc1ccccc1)C(C)C]

in the presence of a catalyst, (b) reacting the product of the hydrogenation reaction of (17) with PTSA monohydrate in the presence of 2-methyltetrahydrofuran, (c) adding

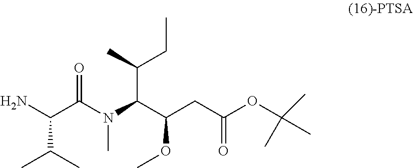 [SMILES: CC[C@H](C)[C@@H]([C@@H](CC(=O)OC(C)(C)C)OC)N(C)C(=O)[C@@H](N)C(C)C]

seed, and (d) isolating

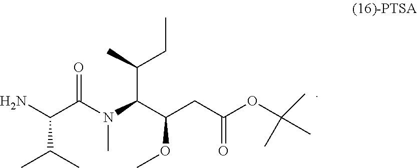 [SMILES: CC[C@H](C)[C@@H]([C@@H](CC(=O)OC(C)(C)C)OC)N(C)C(=O)[C@@H](N)C(C)C]

In some embodiments of the process of making (16)-PTSA, the catalyst used in the hydrogenation reaction of (17) is a Pd catalyst. In some embodiments of the process of making (16)-PTSA, the catalyst used in the hydrogenation reaction of (17) is Pd/C.

In some embodiments of the process of making (16)-PTSA, (17) is hydrogenated in the presence of Pd/C at about 15° C. to about 25° C., at a pressure of not less than about 3.2 bar for a total time of not less than about 3 hours. In some embodiments of the process of making (16)-PTSA, (17) is hydrogenated in the presence of Pd/C at about 10° C. to about 30° C. In some embodiments of the process of making (16)-PTSA, (17) is hydrogenated in the presence of Pd/C at a pressure of not less than about 3.2 bar for a total time of not less than about 3 hours.

In some embodiments of the process of making (16)-PTSA, (17) is hydrogenated in the presence of Pd/C at about 15° C. to about 25° C., at a pressure of not less than 3.2 bar for a total time of not less than about 3 hours, and the reaction mixture is filtered and distilled from isopropyl acetate.

In some embodiments of the process of making (16)-PTSA, a solution of PTSA monohydrate in 2-MeTHF and (16)-PTSA seed are added after the reaction mixture is filtered and distilled from isopropyl acetate, and the mixture is cooled to about 0° C. to about 10° C.

In some embodiments, the process further comprises a process for making

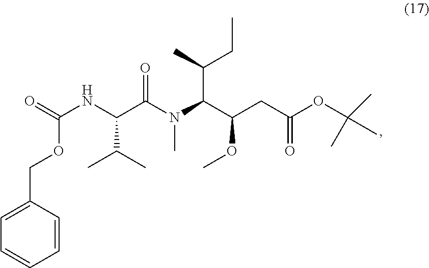 [SMILES: CC[C@H](C)[C@@H]([C@@H](CC(=O)OC(C)(C)C)OC)N(C)C(=O)[C@@H](NC(=O)OCc1ccccc1)C(C)C]

wherein said process comprises reacting

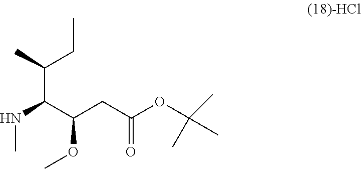 [SMILES: CC[C@H](C)[C@H](NC)[C@@H](CC(=O)OC(C)(C)C)OC]

with Z-Val-OH in the presence of Surfactant TPGS-750-M. In some embodiments of the process of making (17), reacting (18)-HCl with Z-Val-OH to form (17) occurs in the presence of a base. In some embodiments of the process of making (17), reacting (18)-HCl with Z-Val-OH to form (17) occurs in the presence of a tertiary amine base. In some embodiments of the process of making (17), reacting (18)-HCl with Z-Val-OH to form (17) occurs in the presence of N-methylmorpholine. In some embodiments of the process of making (17), reacting (18)-HCl with Z-Val-OH to form (17) occurs in the presence of a coupling reagent. In some embodiments of the process of making (17), reacting (18)-HCl with Z-Val-OH to form (17) occurs in the presence of 4-(4,6-dimethoxy-1,3,5-triazin-2-yl)-4-methylmorpholinium chloride.

In some embodiments of the process of making (17), reacting (18)-HCl with Z-Val-OH to form (17) occurs in the presence of N-methylmorpholine and 4-(4,6-dimethoxy-1,3,5-triazin-2-yl)-4-methylmorpholinium chloride in aqueous TPGS-750-M and isopropyl acetate at about 15° C. to about 25° C. for not less than about 9 hours.

In some embodiments of the process of making (17) comprising reacting (18)-HCl with Z-Val-OH, the reaction mixture is diluted with isopropyl acetate and washed with aqueous tribasic potassium phosphate and aqueous NaCl/HCl to give (17).

In some embodiments, the process provided herein supplies kilogram quantities of maleimidocaproyl monomethyl auristatin F.

In another aspect, provided herein is process for making the HCl salt of

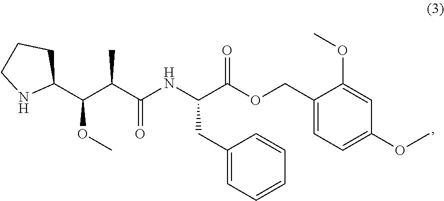 [SMILES: COc1ccc(COC(=O)[C@H](Cc2ccccc2)NC(=O)[C@H](C)[C@@H](OC)[C@@H]2CCCN2)c(OC)c1]

wherein said process comprises reacting

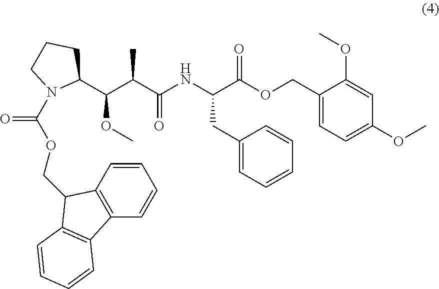 [SMILES: COc1ccc(COC(=O)[C@H](Cc2ccccc2)NC(=O)[C@H](C)[C@@H](OC)[C@@H]2CCCN2C(=O)OCC2c3ccccc3-c3ccccc32)c(OC)c1]

in the presence of a catalytic amount of DBU, and isolating

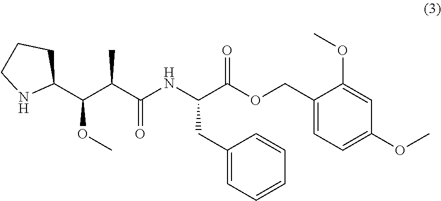 [SMILES: COc1ccc(COC(=O)[C@H](Cc2ccccc2)NC(=O)[C@H](C)[C@@H](OC)[C@@H]2CCCN2)c(OC)c1]

as a salt.

In some embodiments of the process of making the HCl salt of (3),

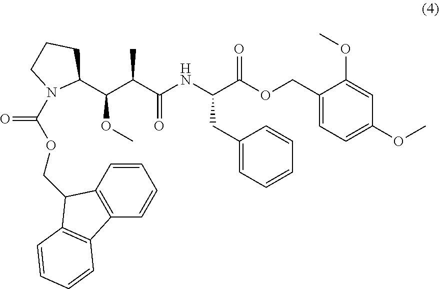 [SMILES: COc1ccc(COC(=O)[C@H](Cc2ccccc2)NC(=O)[C@H](C)[C@@H](OC)[C@@H]2CCCN2C(=O)OCC2c3ccccc3-c3ccccc32)c(OC)c1]

is treated with a thiol. In some embodiments of the process of making the HCl salt of (3), (4) is treated with 1-octanethiol. In some embodiments of the process of making the HCl salt of (3), (3)-HCl is crystallized. In some embodiments of the process of making the HCl salt of (3), 1,4-dioxane and (3)-HCl seed are added prior to isolating (3) as the HCl salt.

In another aspect, provided herein is process for making

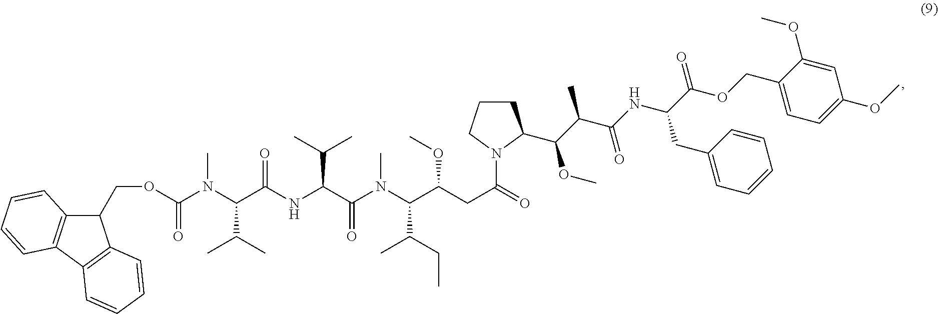 [SMILES: CCC(C)[C@@H]([C@@H](CC(=O)N1CCC[C@H]1[C@H](OC)[C@@H](C)C(=O)N[C@@H](Cc1ccccc1)C(=O)OCc1ccc(OC)cc1OC)OC)N(C)C(=O)[C@@H](NC(=O)[C@H](C(C)C)N(C)C(=O)OCC1c2ccccc2-c2ccccc21)C(C)C]

wherein said process comprises reacting

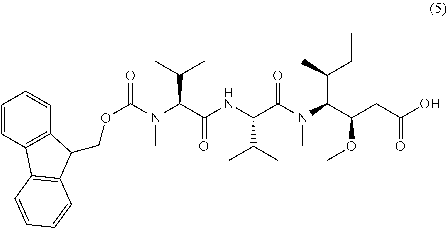 [SMILES: CC[C@H](C)[C@@H]([C@@H](CC(=O)O)OC)N(C)C(=O)[C@@H](NC(=O)[C@H](C(C)C)N(C)C(=O)OCC1c2ccccc2-c2ccccc21)C(C)C]

with the HCl salt of

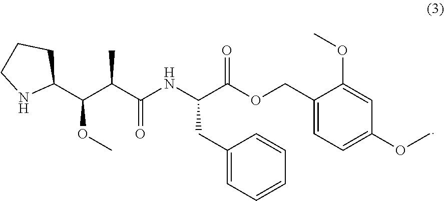 [SMILES: COc1ccc(COC(=O)[C@H](Cc2ccccc2)NC(=O)[C@H](C)[C@@H](OC)[C@@H]2CCCN2)c(OC)c1]

In some embodiments of the process of making (9), (9) is not isolated using column chromatography. In some embodiments of the process of making (9), reacting (5) with (3) occurs in the presence of a tertiary amine base. In some embodiments of the process of making (9), reacting (5) with (3) occurs in the presence of N,N-diisopropylethylamine. In some embodiments of the process of making (9), reacting (5) with (3) occurs in the presence of a coupling reagent. In some embodiments of the process of making (9), reacting (5) with (3) occurs in the presence of O-(benzotriazol-1-yl)-N,N,N′,N′-tetramethyluronium tetrafluoroborate.

In another aspect, provided herein is process for making

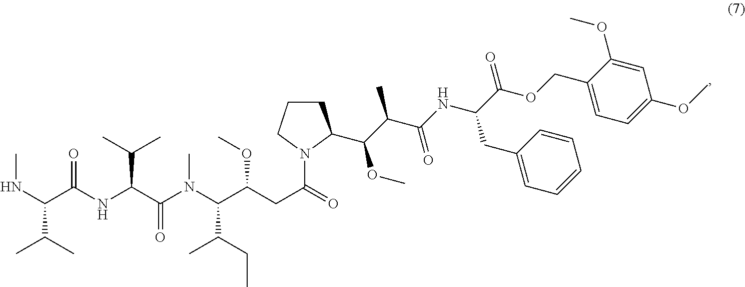 [SMILES: CCC(C)[C@@H]([C@@H](CC(=O)N1CCC[C@H]1[C@H](OC)[C@@H](C)C(=O)N[C@@H](Cc1ccccc1)C(=O)OCc1ccc(OC)cc1OC)OC)N(C)C(=O)[C@@H](NC(=O)[C@@H](NC)C(C)C)C(C)C]

wherein said process comprises reacting

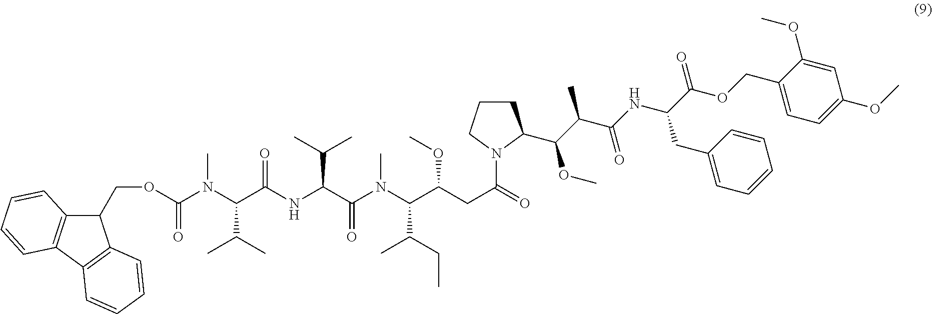 [SMILES: CCC(C)[C@@H]([C@@H](CC(=O)N1CCC[C@H]1[C@H](OC)[C@@H](C)C(=O)N[C@@H](Cc1ccccc1)C(=O)OCc1ccc(OC)cc1OC)OC)N(C)C(=O)[C@@H](NC(=O)[C@H](C(C)C)N(C)C(=O)OCC1c2ccccc2-c2ccccc21)C(C)C]

in the presence of a catalytic amount of DBU. In some embodiments of the process of making (7), (7) is not isolated using column chromatography.

In another aspect, provided herein is process for making

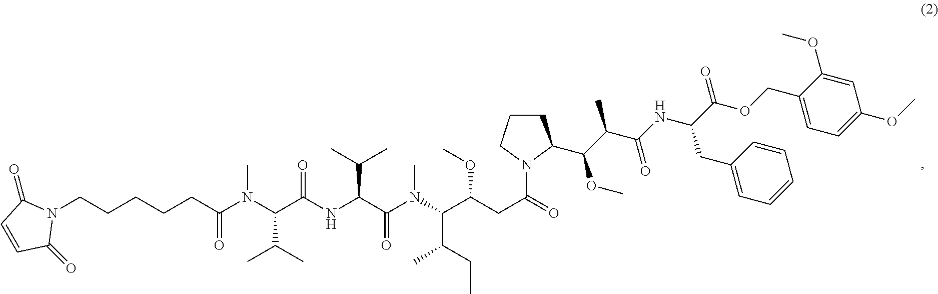 [SMILES: CC[C@H](C)[C@@H]([C@@H](CC(=O)N1CCC[C@H]1[C@H](OC)[C@@H](C)C(=O)N[C@@H](Cc1ccccc1)C(=O)OCc1ccc(OC)cc1OC)OC)N(C)C(=O)[C@@H](NC(=O)[C@H](C(C)C)N(C)C(=O)CCCCCN1C(=O)C=CC1=O)C(C)C]

wherein said process comprises reacting

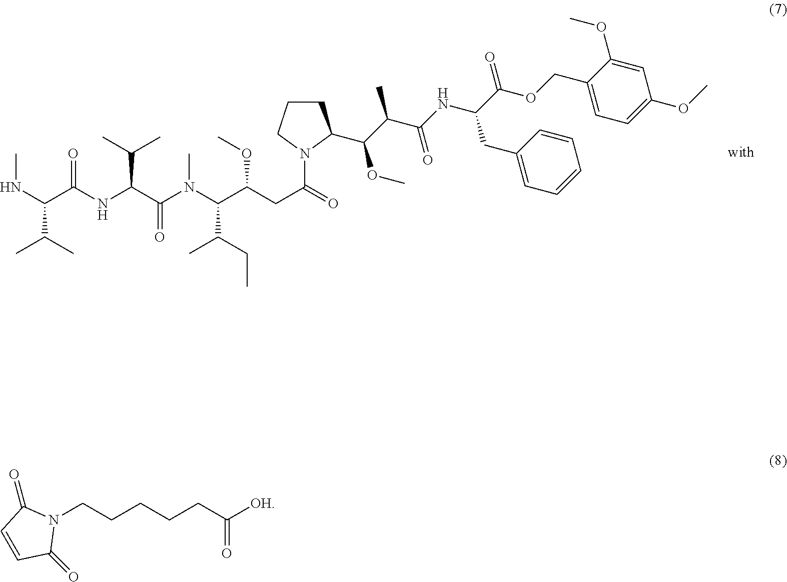 [SMILES: CCC(C)[C@@H]([C@@H](CC(=O)N1CCC[C@H]1[C@H](OC)[C@@H](C)C(=O)N[C@@H](Cc1ccccc1)C(=O)OCc1ccc(OC)cc1OC)OC)N(C)C(=O)[C@@H](NC(=O)[C@@H](NC)C(C)C)C(C)C ; O=C(O)CCCCCN1C(=O)C=CC1=O]

In some embodiments of the process of making (2),

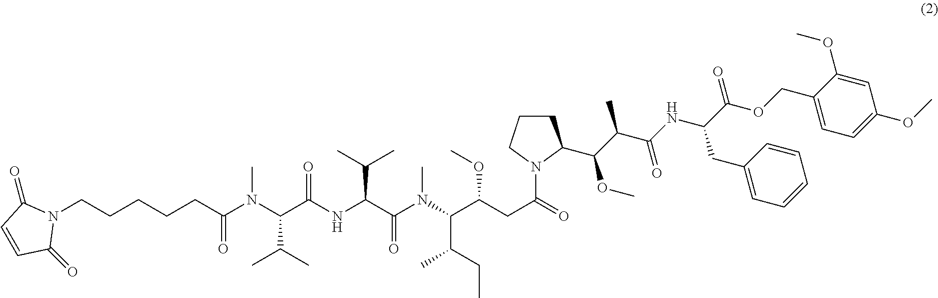 [SMILES: CC[C@H](C)[C@@H]([C@@H](CC(=O)N1CCC[C@H]1[C@H](OC)[C@@H](C)C(=O)N[C@@H](Cc1ccccc1)C(=O)OCc1ccc(OC)cc1OC)OC)N(C)C(=O)[C@@H](NC(=O)[C@H](C(C)C)N(C)C(=O)CCCCCN1C(=O)C=CC1=O)C(C)C]

is not isolated using column chromatography. In some embodiments of the process of making (2), reacting (7) with (8) occurs in the presence of a tertiary amine base. In some embodiments of the process of making (2), reacting (7) with (8) occurs in the presence of N,N-diisopropylethylamine. In some embodiments of the process of making (2), reacting (7) with (8) occurs in the presence of a coupling reagent. In some embodiments of the process of making (2), reacting (7) with (8) occurs in the presence of 2-chloro-1-methylpyridinium iodide.

### Definitions

For use herein, unless clearly indicated otherwise, use of the terms “a”, “an” and the like refers to one or more.

Reference to “about” a value or parameter herein includes (and describes) embodiments that are directed to that value or parameter per se. For example, description referring to “about X” includes description of “X”. In some embodiments, “about” refers to ±20%, ±15%, ±10%, ±5%, ±2%, or ±1% of the stated value.

It is understood that aspects and embodiments described herein as “comprising” include “consisting of” and “consisting essentially of” embodiments.

First Generation (1G) Process to mcMMAF

mcMMAF (1) is a synthetic peptide that consists of natural and unnatural amino acids (Pettit G R, et al., Antineoplastic agents 337. Synthesis of dolastatin 10 structural modifications. Anticancer Drug Des. 1995 October; 10(7):529-44; Pettit G R, et al. Synthesis and cancer cell growth inhibitory studies of dolastatin 15 structural modifications. Anticancer Drug Des. 1998 January; 13(1):47-66. PMID: 9474242, each herein incorporated by reference in its entirety). Many established syntheses often use large amounts of hazardous reagents and solvents, resulting in inefficient processes with often high PMIs (Albert Isidro-Llobet, Martin N. Kenworthy, Subha Mukherjee, Michael E. Kopach, Katarzyna Wegner, Fabrice Gallou, Austin G. Smith, and Frank Roschangar The Journal of Organic Chemistry 2019 84 (8), 4615-462, herein incorporated by reference in its entirety). However, PMI does not address concerns regarding the toxicity or health and safety of the feedstock materials or wastes produced. Life Cycle Assessment (LCA) methodologies have been identified as one of the top ten priority areas for green engineering research. LCA is a standardized methodology, recognized as an international tool able to identify hot spots and to express sustainability (Daniele Cespi, Evan S. Beach, Thomas E. Swarr, Fabrizio Passarini, I. Vassura, Peter J. Dunne, and Paul T. Anastasa Green Chemistry 2015 17 (6), 3390-3400, herein incorporated by reference in its entirety).

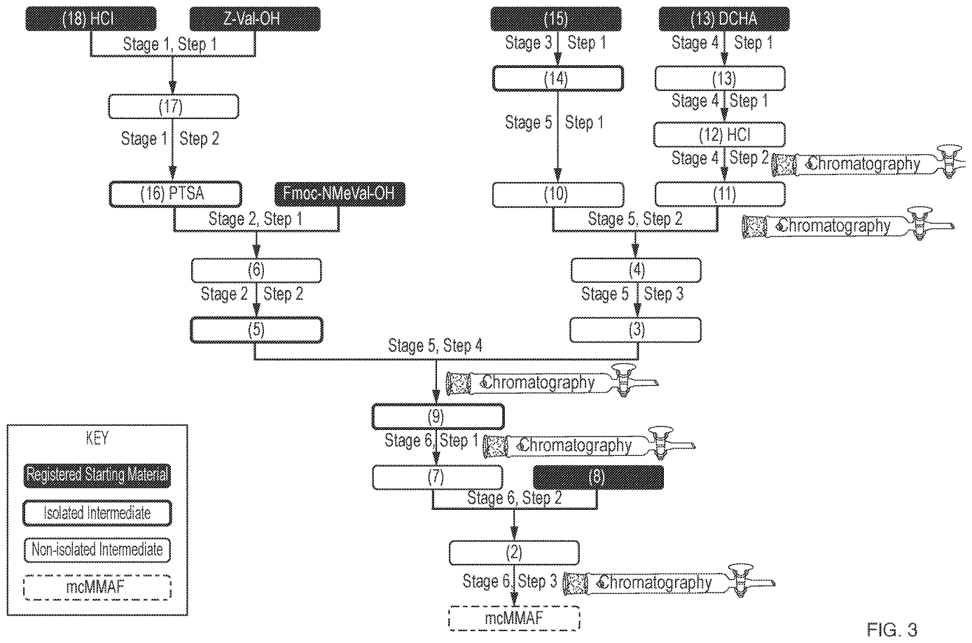

The First Generation (1G) process to mcMMAF was a fit-for-purpose early discovery chemistry route, and a map of this process is depicted in FIG. 3. The process map provides a good overall picture of the 1G process. As shown and due to the molecular complexity of mcMMAF, the process involves six starting materials and a total of fourteen intermediates. A synthetic scheme for the First Generation (1G) process to mcMMAF is shown in Scheme 1.

### Scheme 1

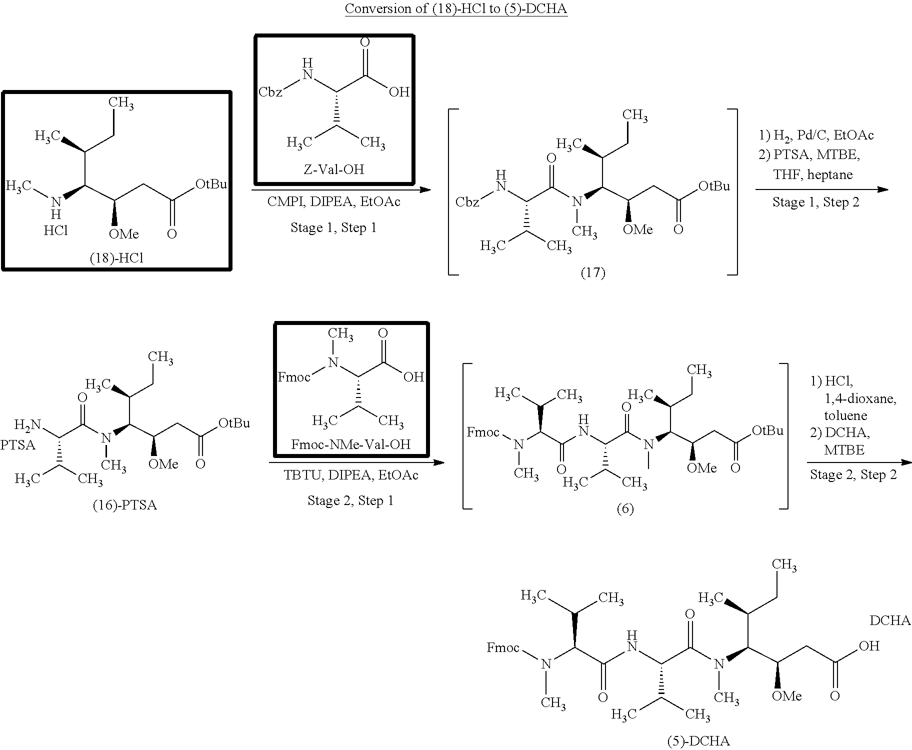

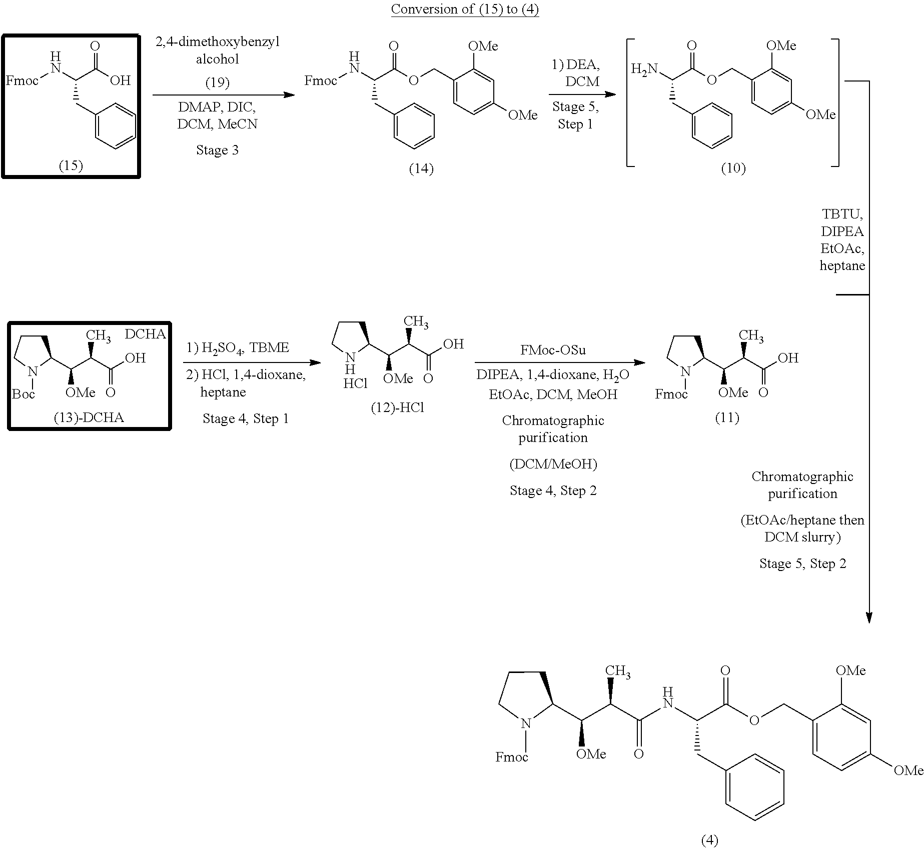

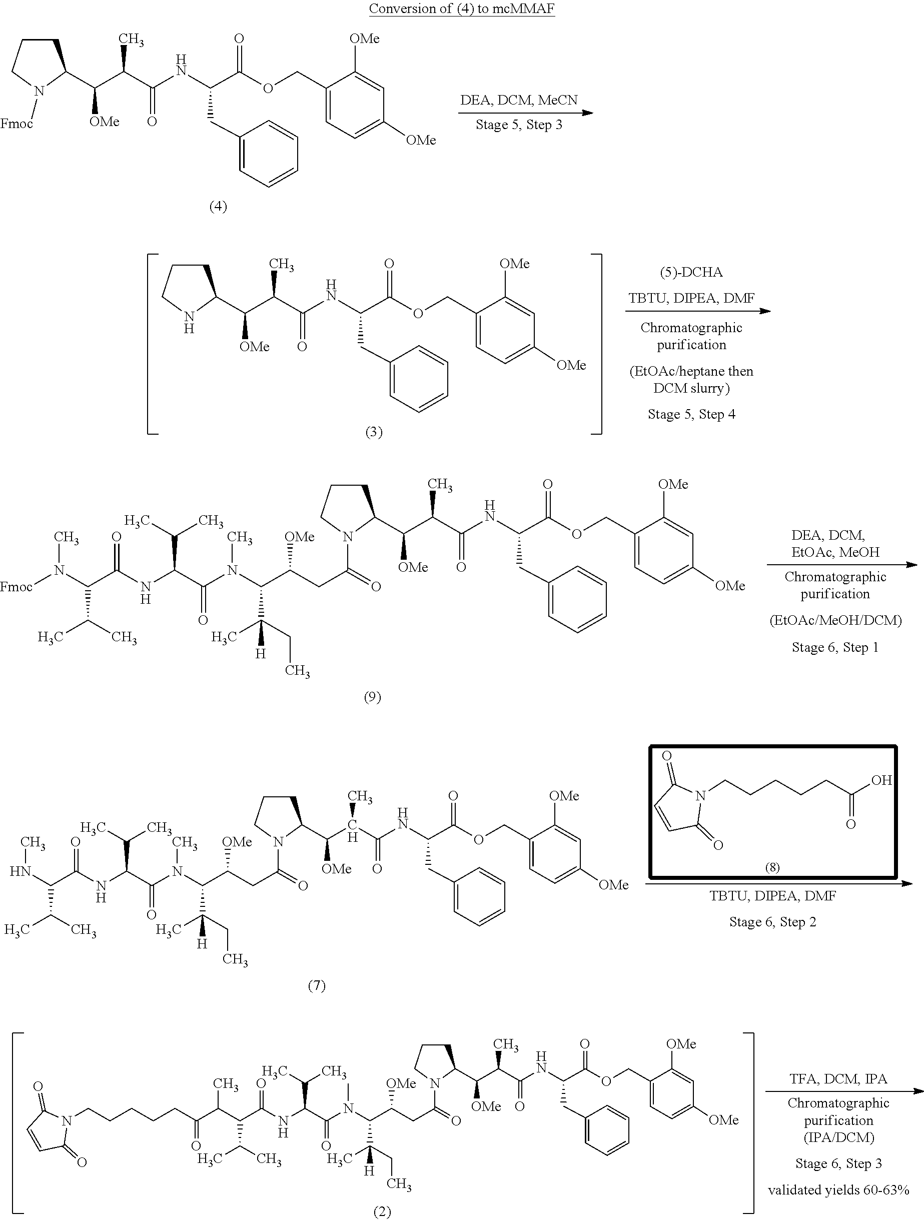

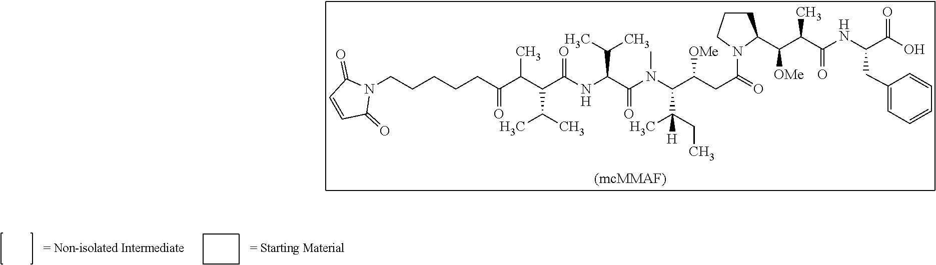

Process Metrics for the 1G Process of mcMMAF were calculated using the American Chemical Society Green Chemistry Institute's Streamlined PMI-LCA tool (Table 1) (https://members.acsgcipr.org/tools-development/pmi-lca-tool/, herein incorporated by reference in its entirety). The overall PMI calculated using this tool for the 1G Process was 11581 per kg of mcMMAF. Solvents account for over 90% of the PMI for this process. The energy consumption for this process was 665325 MJ. The Global Warming Potential (GWP) calculated for this process releases an equivalent amount of 36 tons of CO₂ per Kg of mcMMAF produced.

TABLE 1
Process Metrics for 1G Process Calculated using ACSGCI PMI-LCA Tool.

| Process Metrics per kg API | Total | Reagent | Metal | Solvent | Water |
|---|---|---|---|---|---|
| PMI | 11581.04 | 351.21 | 0.44 | 10503.08 | 726.31 |
| Mass Net (kg) | 23680.37 | 2550.35 | 380.78 | 20748.13 | 1.11 |
| Energy (MJ) | 665325.86 | 45964.11 | 7973.21 | 611374.69 | 13.84 |
| GWP (kg CO2 equiv.) | 33457.66 | 4744.32 | 426.53 | 28286.08 | 0.74 |
| Acidification (kg SO2 equiv.) | 511.70 | 40.03 | 310.65 | 161.01 | 0.00 |
| Eutrophication (kg phosphate equiv.) | 37.94 | 4.46 | 3.06 | 30.42 | 0.00 |
| Water (kg) | 92489.01 | 926.89 | 13524.03 | 77120.51 | 917.58 |

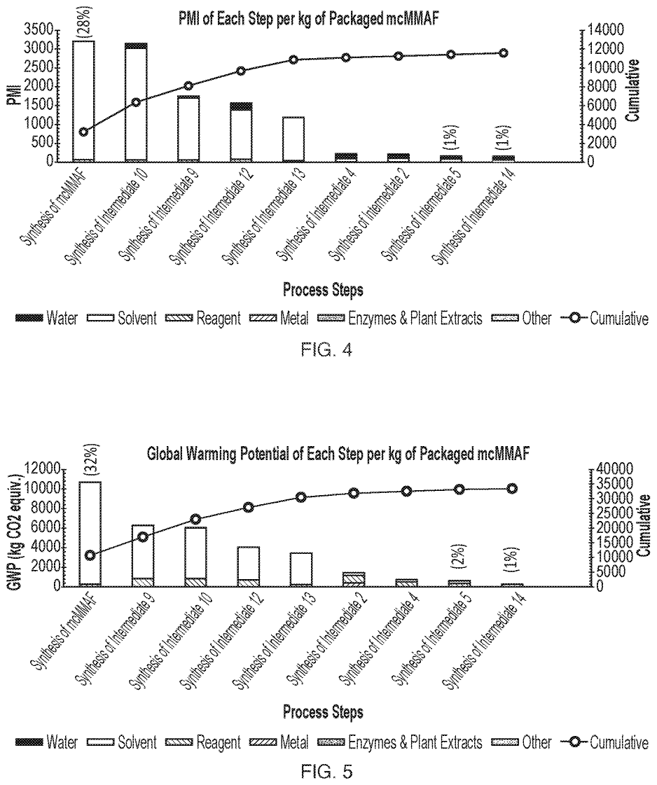

The high PMI correlates well with those intermediates in which column chromatography is used for their purification (FIG. 4). The same correlation can be seen when looking at the GWP numbers broken by major intermediates (FIG. 5).

Second Generation (2G) Process to mcMMAF

An effort was initiated to develop a second-generation (2G) process for mcMMAF, which not only can be more efficient and economical, but also can have a lesser impact on the environment. Scheme 2 illustrates the exemplary second-generation process for mcMMAF (1). In addition to improving the efficiency, the 2G process was able to (i) replace ethyl acetate with environmental benign surfactant TPGS-750-M in Stage 1, step 1; (ii) remove dichloromethane as a reaction and work-up solvent throughout the synthesis in Stage 3, step 1, Stage 5, steps 1 and 3, and Step 6, stages 1 and 3; (iii) remove dioxane as a reaction solvent in Stage 4; (iv) remove DMF as reaction solvent in Stage 6, steps 1 and 2; and (v) remove four-chromatographic separations.

Single-use flash chromatography in the final chromatographic separation was replaced by high pressure chromatographic purification, which reduced solvent consumption and eliminated the need for a new purification stationary phase (cartridge) for every 300 g of crude mcMMAF (1) produced.

Some embodiments relate to improving the existing process by minimizing or avoiding the use of large excesses of organic solvents (e.g., dichloromethane, dioxane, ethyl acetate, heptane, methanol, isopropanol) during the reactions and/or purifications. In addition, some embodiments relate to improving the existing process by identifying solid isolation points that would allow for the removal of several chromatographic purifications without any negative impact to product quality. The process is complex as mcMMAF has 9 stereogenic centers and thus 512 possible diastereoisomers. Finally, due to the known potent toxicity of mcMMAF, special precautions were undertaken during process development in order to safely handle compounds in the process. Despite these challenges, a 2G process was developed. In some embodiments, the process has met quality, efficiency, cost and sustainability goals. In some embodiments, the process does not use dioxane or dichloromethane as solvents. In some embodiments, the process does not use dioxane as solvent. In some embodiments, the process does not use dichloromethane as solvent.

### ENUMERATED EMBODIMENTS

Enumerated Embodiment 1: A process for making maleimidocaproyl monomethyl auristatin F comprising:
(a) converting compound

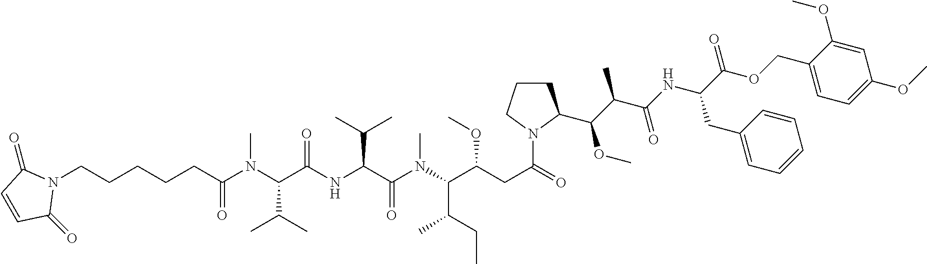 [SMILES: CC[C@H](C)[C@@H]([C@@H](CC(=O)N1CCC[C@H]1[C@H](OC)[C@@H](C)C(=O)N[C@@H](Cc1ccccc1)C(=O)OCc1ccc(OC)cc1OC)OC)N(C)C(=O)[C@@H](NC(=O)[C@H](C(C)C)N(C)C(=O)CCCCCN1C(=O)C=CC1=O)C(C)C]

(2) to maleimidocaproyl monomethyl Auristatin F in the presence of toluene and 2-methyltetrahydrofuran,
(b) an aqueous workup, and
(c) chromatographic purification.
Enumerated Embodiment 2: The process of Enumerated Embodiment 1, further comprising a process for making

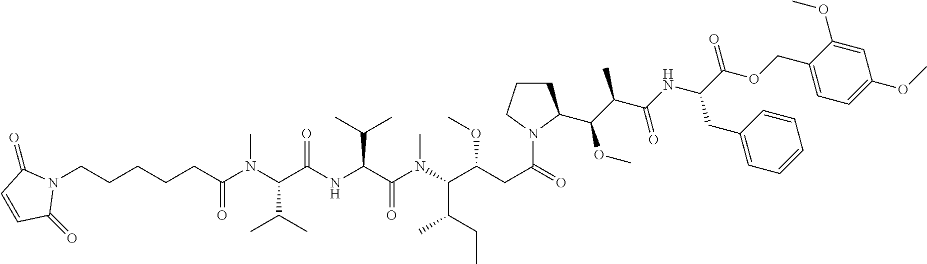 [SMILES: CC[C@H](C)[C@@H]([C@@H](CC(=O)N1CCC[C@H]1[C@H](OC)[C@@H](C)C(=O)N[C@@H](Cc1ccccc1)C(=O)OCc1ccc(OC)cc1OC)OC)N(C)C(=O)[C@@H](NC(=O)[C@H](C(C)C)N(C)C(=O)CCCCCN1C(=O)C=CC1=O)C(C)C]

wherein said process comprises reacting

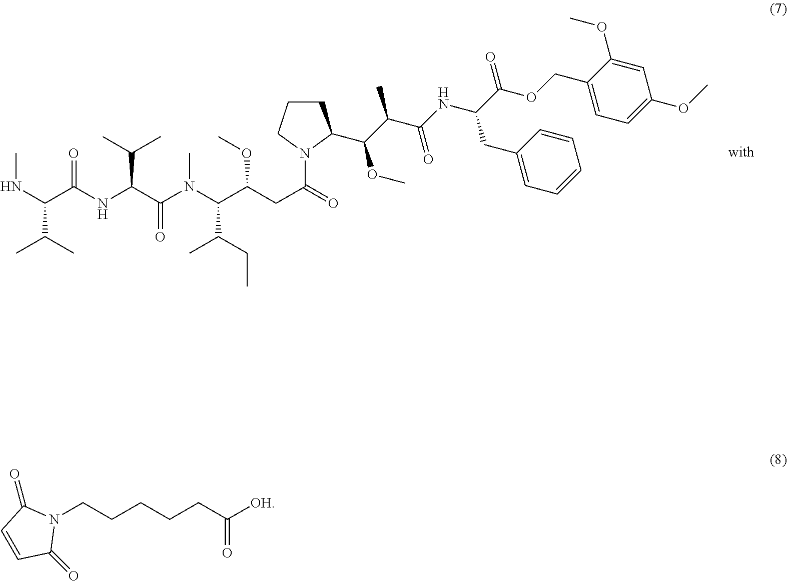 [SMILES: CCC(C)[C@@H]([C@@H](CC(=O)N1CCC[C@H]1[C@H](OC)[C@@H](C)C(=O)N[C@@H](Cc1ccccc1)C(=O)OCc1ccc(OC)cc1OC)OC)N(C)C(=O)[C@@H](NC(=O)[C@@H](NC)C(C)C)C(C)C ; O=C(O)CCCCCN1C(=O)C=CC1=O]

Enumerated Embodiment 3: The process of Enumerated Embodiments 1 or 2, further comprising a process for making

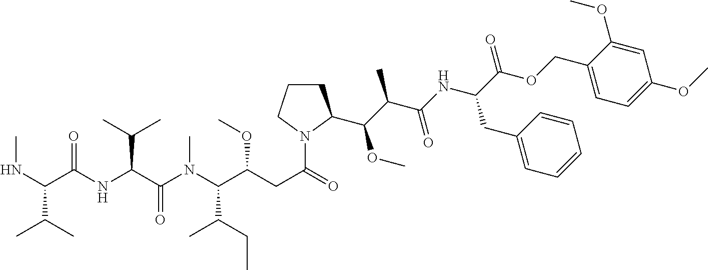 [SMILES: CCC(C)[C@@H]([C@@H](CC(=O)N1CCC[C@H]1[C@H](OC)[C@@H](C)C(=O)N[C@@H](Cc1ccccc1)C(=O)OCc1ccc(OC)cc1OC)OC)N(C)C(=O)[C@@H](NC(=O)[C@@H](NC)C(C)C)C(C)C]

wherein said process comprises reacting

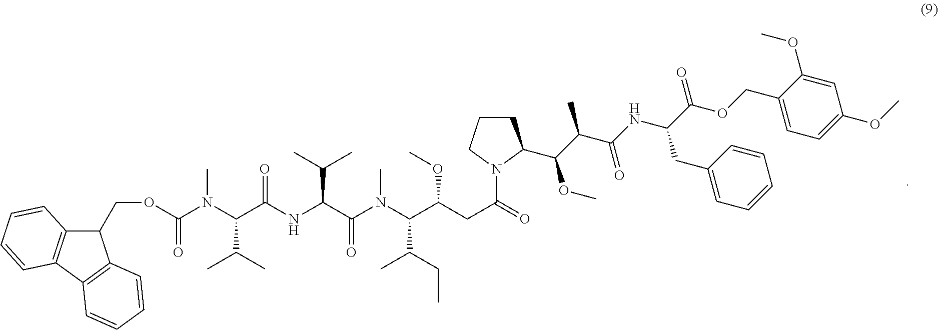 [SMILES: CCC(C)[C@@H]([C@@H](CC(=O)N1CCC[C@H]1[C@H](OC)[C@@H](C)C(=O)N[C@@H](Cc1ccccc1)C(=O)OCc1ccc(OC)cc1OC)OC)N(C)C(=O)[C@@H](NC(=O)[C@H](C(C)C)N(C)C(=O)OCC1c2ccccc2-c2ccccc21)C(C)C]

in the presence of a catalytic amount of DBU.
Enumerated Embodiment 4: The process of Enumerated Embodiment 3, wherein

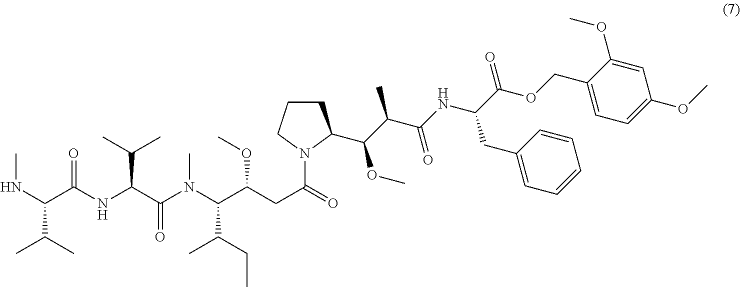 [SMILES: CCC(C)[C@@H]([C@@H](CC(=O)N1CCC[C@H]1[C@H](OC)[C@@H](C)C(=O)N[C@@H](Cc1ccccc1)C(=O)OCc1ccc(OC)cc1OC)OC)N(C)C(=O)[C@@H](NC(=O)[C@@H](NC)C(C)C)C(C)C]

is not isolated using column chromatography.
Enumerated Embodiment 5: The process of any of Enumerated Embodiments 1-4, further comprising a process for making

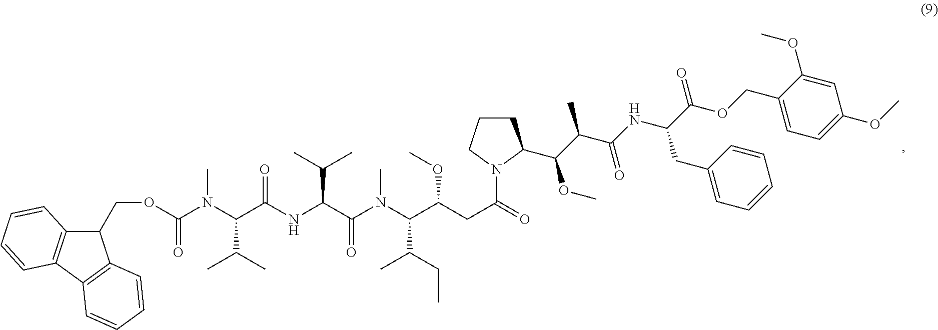 [SMILES: CCC(C)[C@@H]([C@@H](CC(=O)N1CCC[C@H]1[C@H](OC)[C@@H](C)C(=O)N[C@@H](Cc1ccccc1)C(=O)OCc1ccc(OC)cc1OC)OC)N(C)C(=O)[C@@H](NC(=O)[C@H](C(C)C)N(C)C(=O)OCC1c2ccccc2-c2ccccc21)C(C)C]

wherein said process comprises reacting

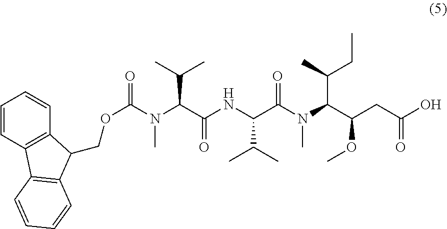 [SMILES: CC[C@H](C)[C@@H]([C@@H](CC(=O)O)OC)N(C)C(=O)[C@@H](NC(=O)[C@H](C(C)C)N(C)C(=O)OCC1c2ccccc2-c2ccccc21)C(C)C]

with the HCl salt of

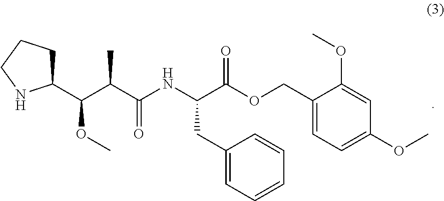 [SMILES: COc1ccc(COC(=O)[C@H](Cc2ccccc2)NC(=O)[C@H](C)[C@@H](OC)[C@@H]2CCCN2)c(OC)c1]

Enumerated Embodiment 6: The process of Enumerated Embodiment 5, wherein

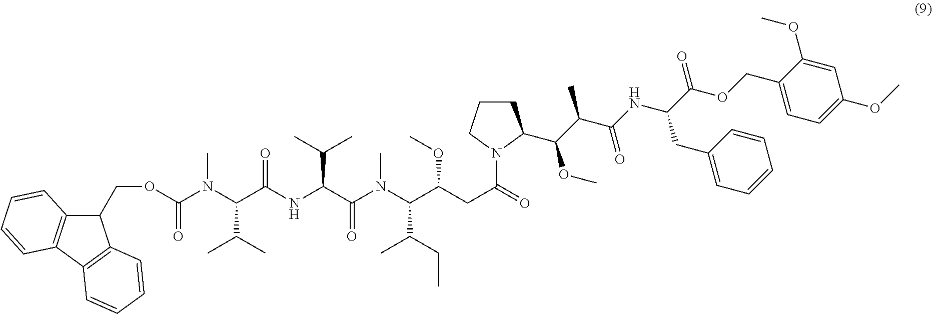 [SMILES: CCC(C)[C@@H]([C@@H](CC(=O)N1CCC[C@H]1[C@H](OC)[C@@H](C)C(=O)N[C@@H](Cc1ccccc1)C(=O)OCc1ccc(OC)cc1OC)OC)N(C)C(=O)[C@@H](NC(=O)[C@H](C(C)C)N(C)C(=O)OCC1c2ccccc2-c2ccccc21)C(C)C]

is not isolated using chromatography.
Enumerated Embodiment 7: The process of any of Enumerated Embodiments 1-6, further comprising a process for making the HCl salt of

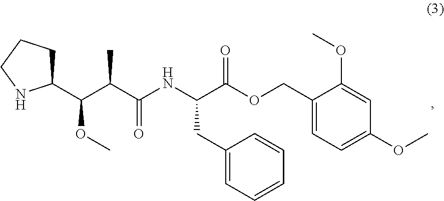 [SMILES: COc1ccc(COC(=O)[C@H](Cc2ccccc2)NC(=O)[C@H](C)[C@@H](OC)[C@@H]2CCCN2)c(OC)c1]

wherein said process comprises:
(a) reacting

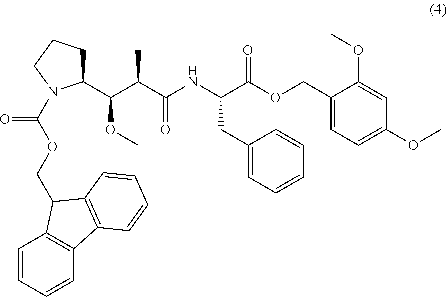 [SMILES: COc1ccc(COC(=O)[C@H](Cc2ccccc2)NC(=O)[C@H](C)[C@@H](OC)[C@@H]2CCCN2C(=O)OCC2c3ccccc3-c3ccccc32)c(OC)c1]

in the presence of a catalytic amount of DBU, and
(b) isolating

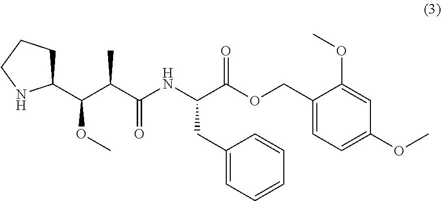 [SMILES: COc1ccc(COC(=O)[C@H](Cc2ccccc2)NC(=O)[C@H](C)[C@@H](OC)[C@@H]2CCCN2)c(OC)c1]

as a salt.
Enumerated Embodiment 8: The process of any of Enumerated Embodiments 1-7, further comprising a process for making

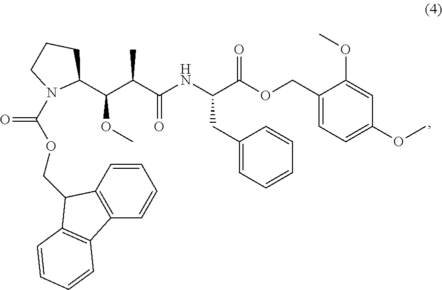 [SMILES: COc1ccc(COC(=O)[C@H](Cc2ccccc2)NC(=O)[C@H](C)[C@@H](OC)[C@@H]2CCCN2C(=O)OCC2c3ccccc3-c3ccccc32)c(OC)c1]

wherein said process comprises reacting the HCl salt of

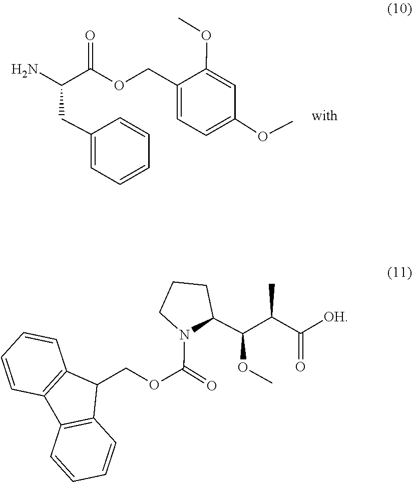 [SMILES: COc1ccc(COC(=O)[C@@H](N)Cc2ccccc2)c(OC)c1 ; CO[C@H]([C@@H](C)C(=O)O)[C@@H]1CCCN1C(=O)OCC1c2ccccc2-c2ccccc21]

Enumerated Embodiment 9: The process of Enumerated Embodiment 8, wherein

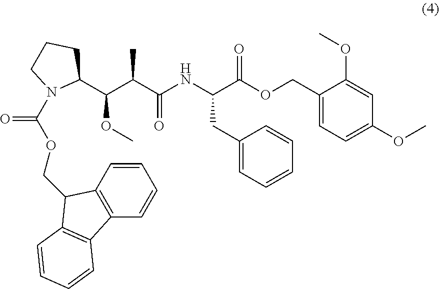 [SMILES: COc1ccc(COC(=O)[C@H](Cc2ccccc2)NC(=O)[C@H](C)[C@@H](OC)[C@@H]2CCCN2C(=O)OCC2c3ccccc3-c3ccccc32)c(OC)c1]

is not isolated using column chromatography.
Enumerated Embodiment 10: The process of any of Enumerated Embodiments 1-9, further comprising a process for making

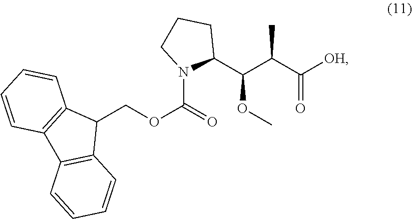 [SMILES: CO[C@H]([C@@H](C)C(=O)O)[C@@H]1CCCN1C(=O)OCC1c2ccccc2-c2ccccc21]

wherein said process comprises reacting the HCl salt of

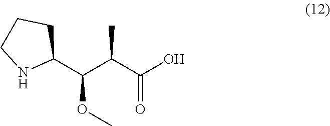

in the presence of water and dimethylformamide.
Enumerated Embodiment 11: The process of Enumerated Embodiment 10, wherein

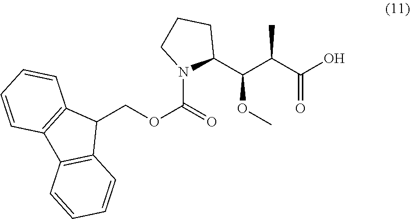 [SMILES: CO[C@H]([C@@H](C)C(=O)O)[C@@H]1CCCN1C(=O)OCC1c2ccccc2-c2ccccc21]

is not isolated using column chromatography.
Enumerated Embodiment 12: The process of any of Enumerated Embodiments 1-11, further comprising a process for making the HCl salt of

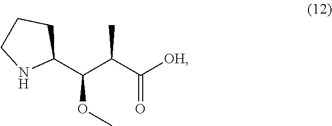

wherein
(a) reacting

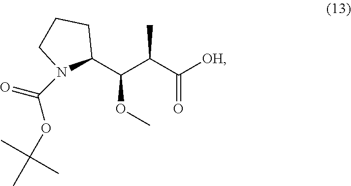 [SMILES: CO[C@H]([C@@H](C)C(=O)O)[C@@H]1CCCN1C(=O)OC(C)(C)C]

and
(b) isolating

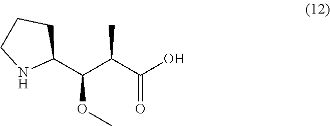

as an HCl salt.
Enumerated Embodiment 13: The process of any of Enumerated Embodiments 1-12, further comprising a process for making

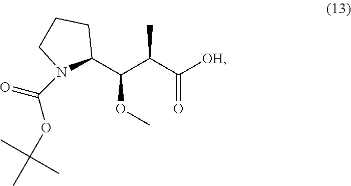 [SMILES: CO[C@H]([C@@H](C)C(=O)O)[C@@H]1CCCN1C(=O)OC(C)(C)C]

wherein said process comprises converting

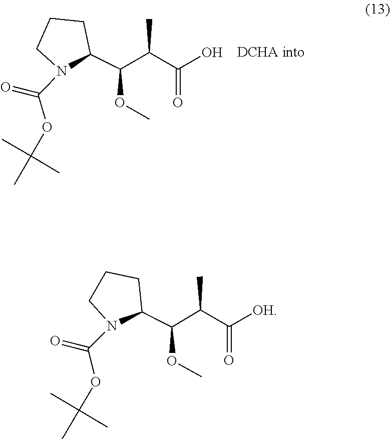 [SMILES: CO[C@H]([C@@H](C)C(=O)O)[C@@H]1CCCN1C(=O)OC(C)(C)C ; CO[C@H]([C@@H](C)C(=O)O)[C@@H]1CCCN1C(=O)OC(C)(C)C]

Enumerated Embodiment 14: The process of any of Enumerated Embodiments 1-13, further comprising a process for making

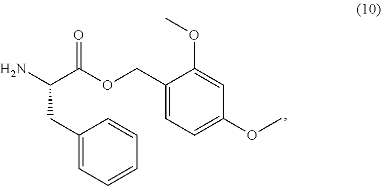 [SMILES: COc1ccc(COC(=O)[C@@H](N)Cc2ccccc2)c(OC)c1]

wherein said process comprises:
(a) reacting

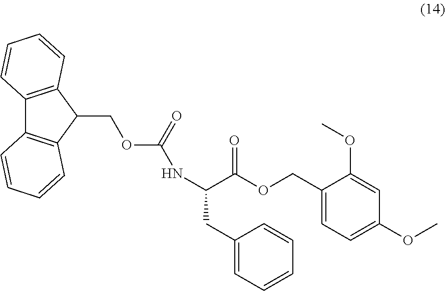 [SMILES: COc1ccc(COC(=O)[C@H](Cc2ccccc2)NC(=O)OCC2c3ccccc3-c3ccccc32)c(OC)c1]

in the presence of a catalytic amount of DBU, and
(b) isolating

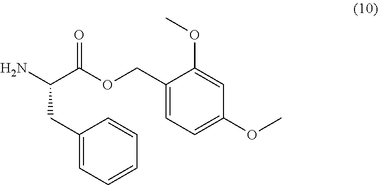 [SMILES: COc1ccc(COC(=O)[C@@H](N)Cc2ccccc2)c(OC)c1]

as an HCl salt.
Enumerated Embodiment 15: The process of any of Enumerated Embodiments 1-14, further comprising a process for making

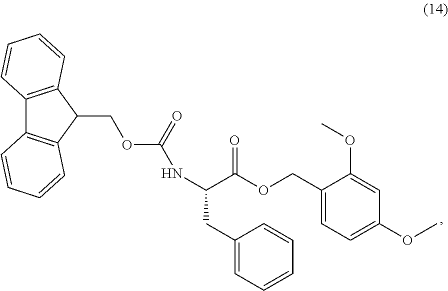 [SMILES: COc1ccc(COC(=O)[C@H](Cc2ccccc2)NC(=O)OCC2c3ccccc3-c3ccccc32)c(OC)c1]

wherein said process comprises converting

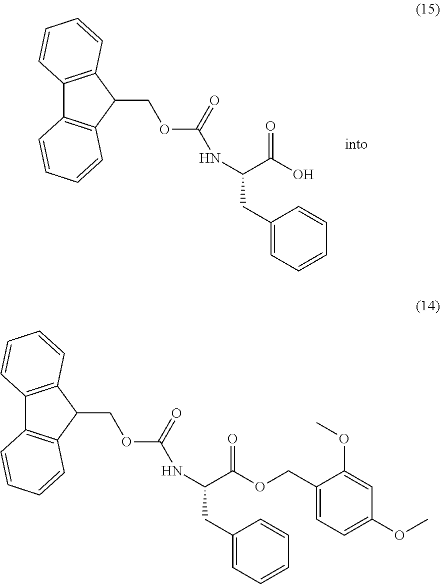 [SMILES: O=C(N[C@@H](Cc1ccccc1)C(=O)O)OCC1c2ccccc2-c2ccccc21 ; COc1ccc(COC(=O)[C@H](Cc2ccccc2)NC(=O)OCC2c3ccccc3-c3ccccc32)c(OC)c1]

in a solvent other than dichloromethane.
Enumerated Embodiment 16: The process of any of Enumerated Embodiments 1-15, further comprising a process for making

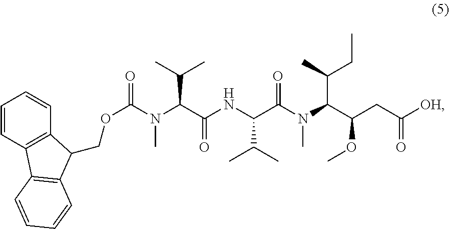 [SMILES: CC[C@H](C)[C@@H]([C@@H](CC(=O)O)OC)N(C)C(=O)[C@@H](NC(=O)[C@H](C(C)C)N(C)C(=O)OCC1c2ccccc2-c2ccccc21)C(C)C]

wherein said process comprises:
(a) reacting

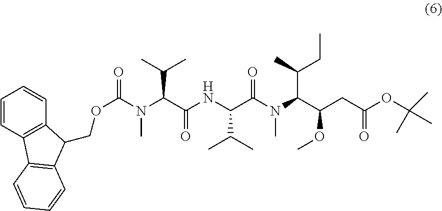 [SMILES: CC[C@H](C)[C@@H]([C@@H](CC(=O)OC(C)(C)C)OC)N(C)C(=O)[C@@H](NC(=O)[C@H](C(C)C)N(C)C(=O)OCC1c2ccccc2-c2ccccc21)C(C)C]

in the presence of aqueous sulfuric acid, and
(b) isolating

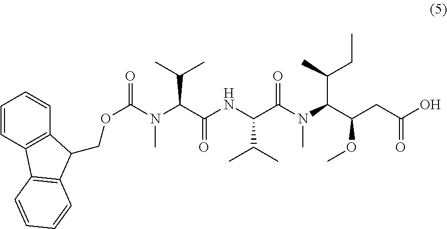 [SMILES: CC[C@H](C)[C@@H]([C@@H](CC(=O)O)OC)N(C)C(=O)[C@@H](NC(=O)[C@H](C(C)C)N(C)C(=O)OCC1c2ccccc2-c2ccccc21)C(C)C]

without a workup.
Enumerated Embodiment 17: The process of any of Enumerated Embodiments 1-16, further comprising a process for making

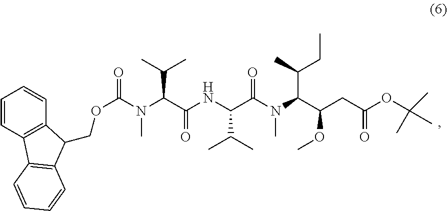 [SMILES: CC[C@H](C)[C@@H]([C@@H](CC(=O)OC(C)(C)C)OC)N(C)C(=O)[C@@H](NC(=O)[C@H](C(C)C)N(C)C(=O)OCC1c2ccccc2-c2ccccc21)C(C)C]

wherein said process comprises reacting

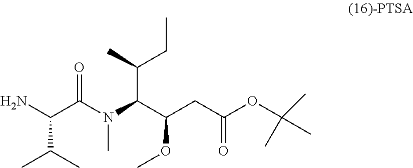 [SMILES: CC[C@H](C)[C@@H]([C@@H](CC(=O)OC(C)(C)C)OC)N(C)C(=O)[C@@H](N)C(C)C]

with Fmoc-NMeVal-OH.
Enumerated Embodiment 18: The process of any of Enumerated Embodiments 1-17, further comprising a process for making

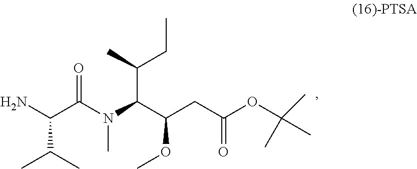 [SMILES: CC[C@H](C)[C@@H]([C@@H](CC(=O)OC(C)(C)C)OC)N(C)C(=O)[C@@H](N)C(C)C]

wherein said process comprises reacting

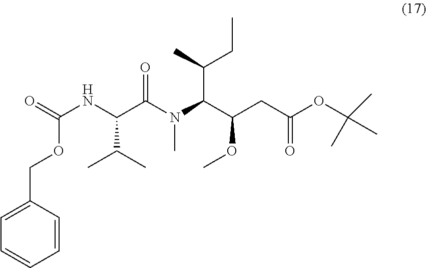 [SMILES: CC[C@H](C)[C@@H]([C@@H](CC(=O)OC(C)(C)C)OC)N(C)C(=O)[C@@H](NC(=O)OCc1ccccc1)C(C)C]

in the presence of 2-methyltetrahydrofuran.
Enumerated Embodiment 19: The process of any of Enumerated Embodiments 1-18, further comprising a process for making

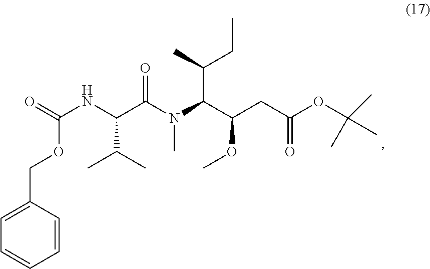 [SMILES: CC[C@H](C)[C@@H]([C@@H](CC(=O)OC(C)(C)C)OC)N(C)C(=O)[C@@H](NC(=O)OCc1ccccc1)C(C)C]

wherein said process comprises reacting

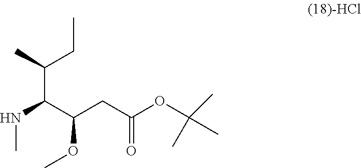 [SMILES: CC[C@H](C)[C@H](NC)[C@@H](CC(=O)OC(C)(C)C)OC]

with Z-Val-OH in the presence of Surfactant TPGS-750-M.

Enumerated Embodiment 20: The process of any of Enumerated Embodiments 1-19, wherein said process supplies kilogram quantities of maleimidocaproyl monomethyl auristatin F.

### EXAMPLES

The following synthetic reaction schemes, which are detailed in the Schemes and Examples, are merely illustrative of some of the methods by which the compounds of the present disclosure, or an embodiment or aspect thereof, can be synthesized. Various modifications to these synthetic reaction schemes can be made, as will be apparent to those of ordinary skill in the art.

The starting materials and the intermediates of the synthetic reaction schemes can be isolated and purified if desired using conventional techniques, including, but not limited to, filtration, distillation, crystallization, chromatography, and the like. Such materials can be characterized using conventional means, including physical constants and spectral data.

Although certain exemplary embodiments are depicted and described herein, the compounds of the present disclosure, or any variation or embodiment thereof, may be prepared using appropriate starting materials according to the methods described generally herein and/or by methods available to one of ordinary skill in the art.

As depicted in the Schemes and Examples below, in certain exemplary embodiments, compounds, or any variation or embodiment thereof, as described elsewhere herein, or a stereoisomer or tautomer thereof, or a pharmaceutically acceptable salt of any of the foregoing, are prepared according to the general procedures. The general methods below, and other methods known to synthetic chemists of ordinary skill in the art, can be applied to all formulae, variations, embodiments, and species described herein.

### Example 1

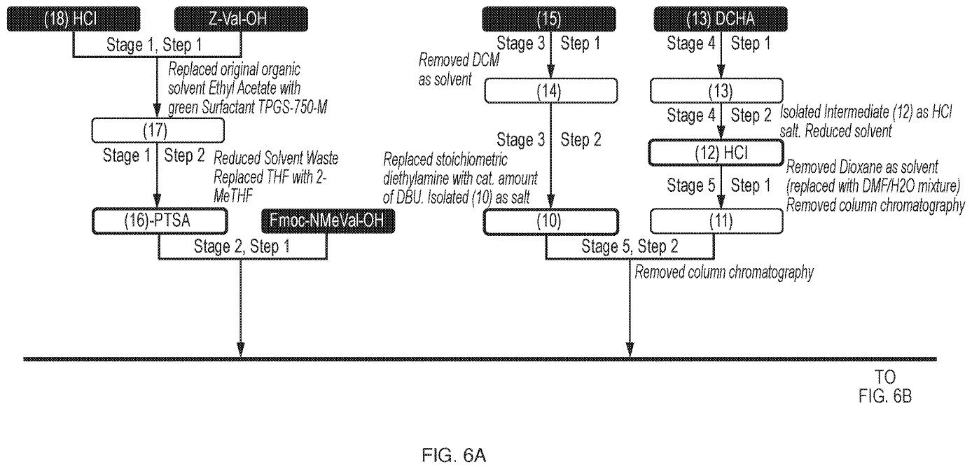

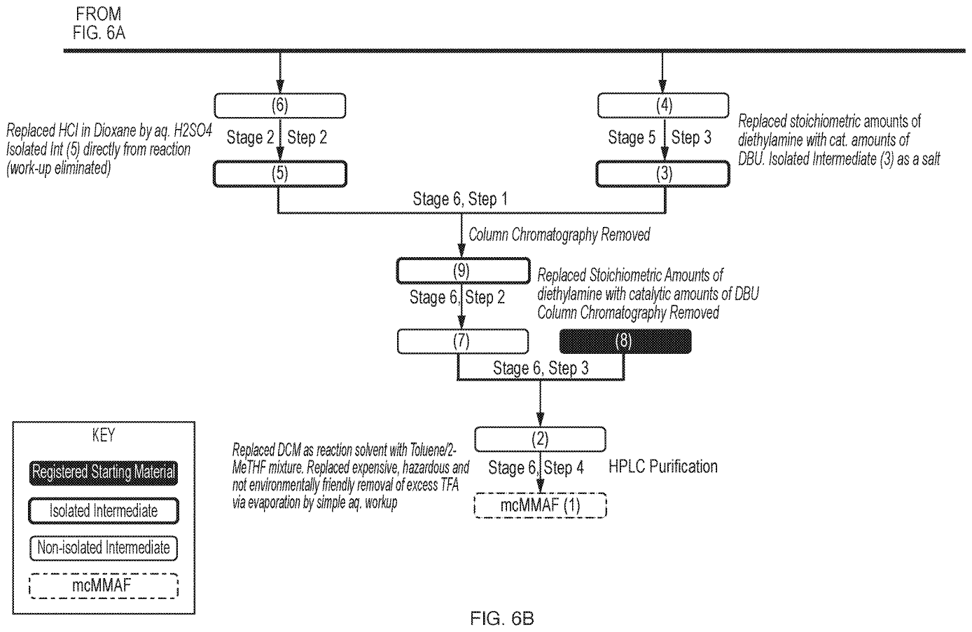

FIG. 6 illustrates the 2G process map.
Stage 1, Step 1:

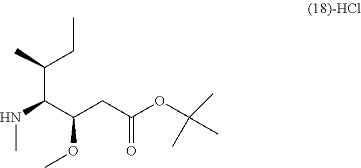 [SMILES: CC[C@H](C)[C@H](NC)[C@@H](CC(=O)OC(C)(C)C)OC]

was reacted with Z-Val-OH in the presence of Surfactant TPGS-750-M to obtain

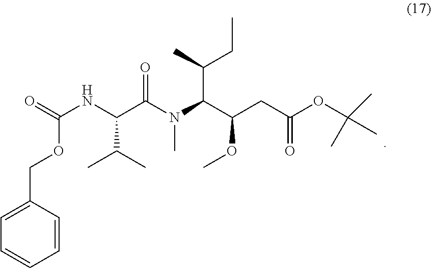 [SMILES: CC[C@H](C)[C@@H]([C@@H](CC(=O)OC(C)(C)C)OC)N(C)C(=O)[C@@H](NC(=O)OCc1ccccc1)C(C)C]

Stage 1, Step 2:

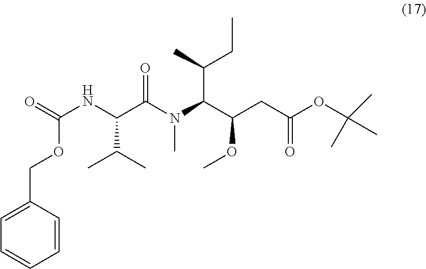 [SMILES: CC[C@H](C)[C@@H]([C@@H](CC(=O)OC(C)(C)C)OC)N(C)C(=O)[C@@H](NC(=O)OCc1ccccc1)C(C)C]

was converted to

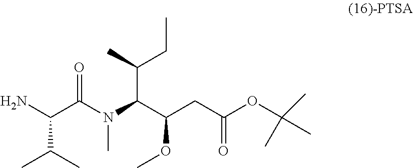 [SMILES: CC[C@H](C)[C@@H]([C@@H](CC(=O)OC(C)(C)C)OC)N(C)C(=O)[C@@H](N)C(C)C]

in the presence of 2-methyltetrahydrofuran (2-MeTHF).
Stage 2, Step 1:

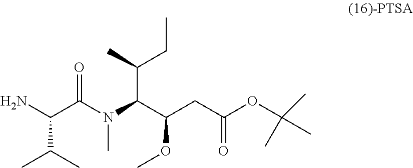 [SMILES: CC[C@H](C)[C@@H]([C@@H](CC(=O)OC(C)(C)C)OC)N(C)C(=O)[C@@H](N)C(C)C]

was reacted with Fmoc-NMeVal-OH to provide

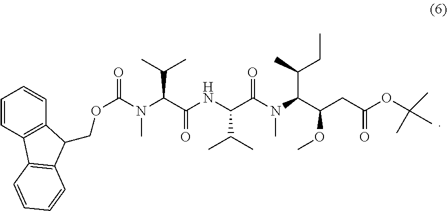 [SMILES: CC[C@H](C)[C@@H]([C@@H](CC(=O)OC(C)(C)C)OC)N(C)C(=O)[C@@H](NC(=O)[C@H](C(C)C)N(C)C(=O)OCC1c2ccccc2-c2ccccc21)C(C)C]

Stage 2, Step 2:

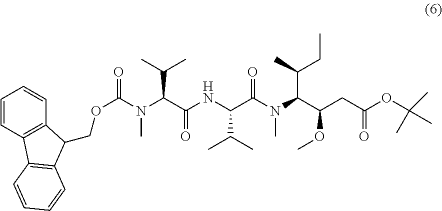 [SMILES: CC[C@H](C)[C@@H]([C@@H](CC(=O)OC(C)(C)C)OC)N(C)C(=O)[C@@H](NC(=O)[C@H](C(C)C)N(C)C(=O)OCC1c2ccccc2-c2ccccc21)C(C)C]

was converted to

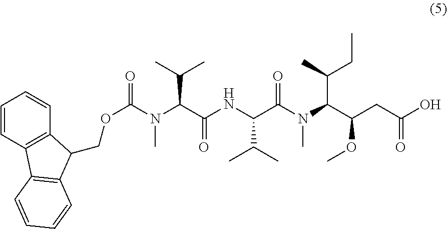 [SMILES: CC[C@H](C)[C@@H]([C@@H](CC(=O)O)OC)N(C)C(=O)[C@@H](NC(=O)[C@H](C(C)C)N(C)C(=O)OCC1c2ccccc2-c2ccccc21)C(C)C]

in the presence of aqueous sulfuric acid.

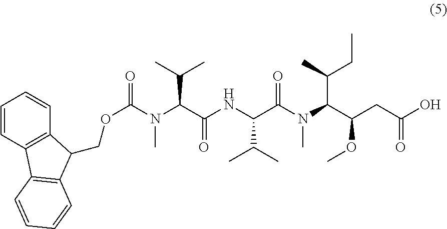 [SMILES: CC[C@H](C)[C@@H]([C@@H](CC(=O)O)OC)N(C)C(=O)[C@@H](NC(=O)[C@H](C(C)C)N(C)C(=O)OCC1c2ccccc2-c2ccccc21)C(C)C]

was isolated directly from the reaction without work-up.
Stage 3, Step 1:

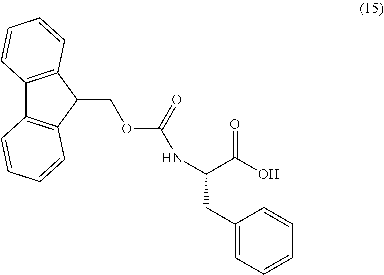 [SMILES: O=C(N[C@@H](Cc1ccccc1)C(=O)O)OCC1c2ccccc2-c2ccccc21]

was converted to

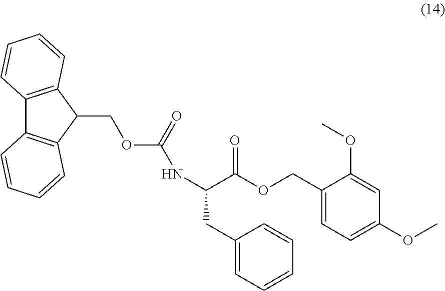 [SMILES: COc1ccc(COC(=O)[C@H](Cc2ccccc2)NC(=O)OCC2c3ccccc3-c3ccccc32)c(OC)c1]

without using dichloromethane (DCM) as solvent.
Stage 3, Step 2:

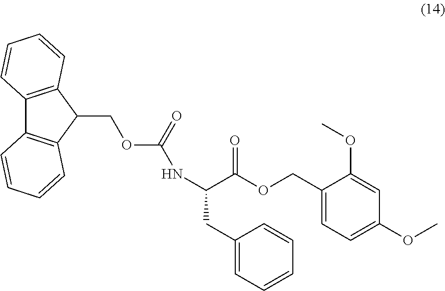 [SMILES: COc1ccc(COC(=O)[C@H](Cc2ccccc2)NC(=O)OCC2c3ccccc3-c3ccccc32)c(OC)c1]

was converted to the HCl salt

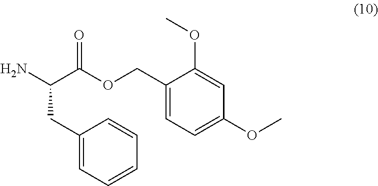 [SMILES: COc1ccc(COC(=O)[C@@H](N)Cc2ccccc2)c(OC)c1]

using catalytic amounts of diazabicycloundecane (DBU).
Stage 4, Step 1:

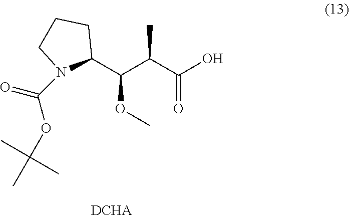 [SMILES: CO[C@H]([C@@H](C)C(=O)O)[C@@H]1CCCN1C(=O)OC(C)(C)C]

was converted to

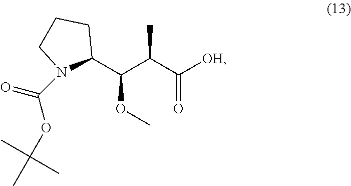 [SMILES: CO[C@H]([C@@H](C)C(=O)O)[C@@H]1CCCN1C(=O)OC(C)(C)C]

Stage 4, Step 2:

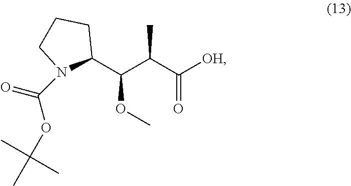 [SMILES: CO[C@H]([C@@H](C)C(=O)O)[C@@H]1CCCN1C(=O)OC(C)(C)C]

was converted to the HCl salt of

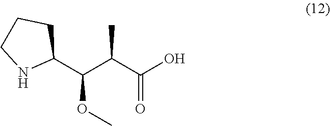

using reduced solvent, and

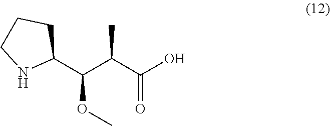

was isolated as the HCl salt.
Stage 5, Step 1: The HCl salt of

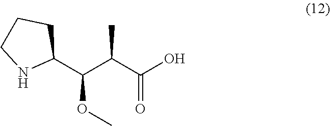

was converted to

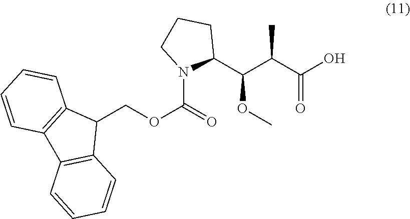 [SMILES: CO[C@H]([C@@H](C)C(=O)O)[C@@H]1CCCN1C(=O)OCC1c2ccccc2-c2ccccc21]

in the presence of dimethylformamide (DMF)/water, and

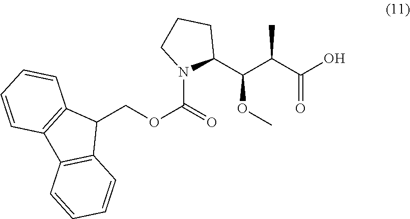 [SMILES: CO[C@H]([C@@H](C)C(=O)O)[C@@H]1CCCN1C(=O)OCC1c2ccccc2-c2ccccc21]

was isolated without using chromatography.
Stage 5, Step 2: The HCl salt of

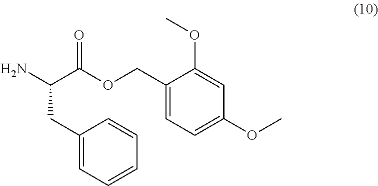 [SMILES: COc1ccc(COC(=O)[C@@H](N)Cc2ccccc2)c(OC)c1]

(from Stage 3, Step 2) was reacted with

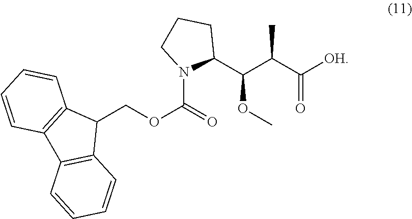 [SMILES: CO[C@H]([C@@H](C)C(=O)O)[C@@H]1CCCN1C(=O)OCC1c2ccccc2-c2ccccc21]

(from Stage 5, Step 1) to provide

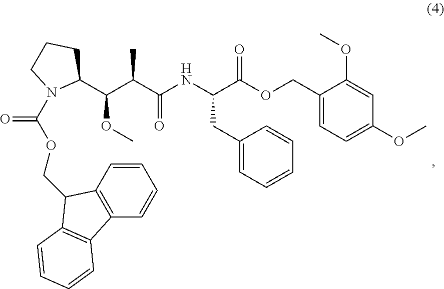 [SMILES: COc1ccc(COC(=O)[C@H](Cc2ccccc2)NC(=O)[C@H](C)[C@@H](OC)[C@@H]2CCCN2C(=O)OCC2c3ccccc3-c3ccccc32)c(OC)c1]

which was isolated without using chromatography.
Stage 5, Step 3:

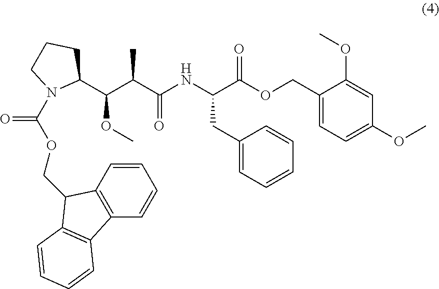 [SMILES: COc1ccc(COC(=O)[C@H](Cc2ccccc2)NC(=O)[C@H](C)[C@@H](OC)[C@@H]2CCCN2C(=O)OCC2c3ccccc3-c3ccccc32)c(OC)c1]

was converted to the HCl salt

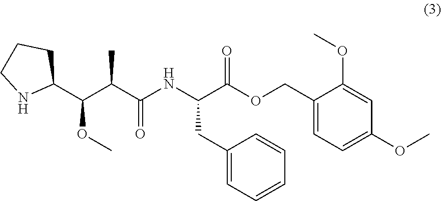 [SMILES: COc1ccc(COC(=O)[C@H](Cc2ccccc2)NC(=O)[C@H](C)[C@@H](OC)[C@@H]2CCCN2)c(OC)c1]

using catalytic amounts of DBU, and

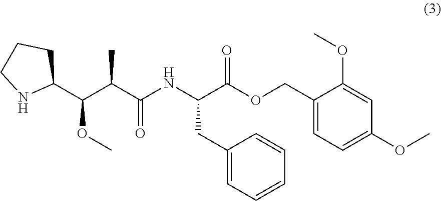 [SMILES: COc1ccc(COC(=O)[C@H](Cc2ccccc2)NC(=O)[C@H](C)[C@@H](OC)[C@@H]2CCCN2)c(OC)c1]

was isolated as the HCl salt.
Stage 6, Step 1:

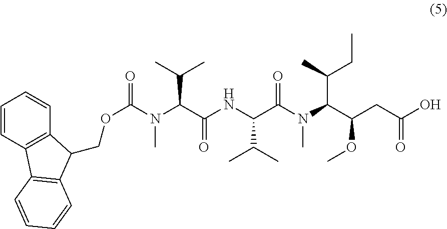 [SMILES: CC[C@H](C)[C@@H]([C@@H](CC(=O)O)OC)N(C)C(=O)[C@@H](NC(=O)[C@H](C(C)C)N(C)C(=O)OCC1c2ccccc2-c2ccccc21)C(C)C]

(from Stage 2, Step 2) was reacted with the HCl salt of

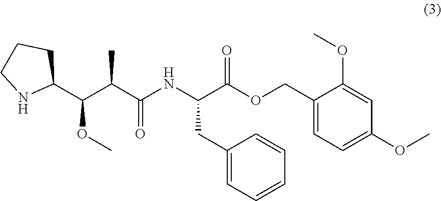 [SMILES: COc1ccc(COC(=O)[C@H](Cc2ccccc2)NC(=O)[C@H](C)[C@@H](OC)[C@@H]2CCCN2)c(OC)c1]

(from Stage 5, Step 3) to obtain

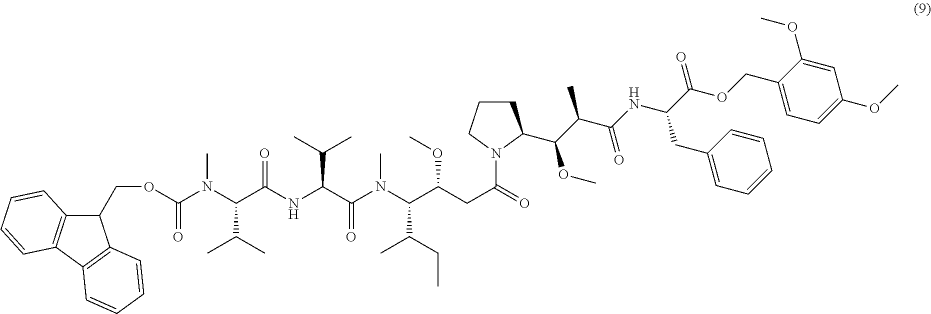 [SMILES: CCC(C)[C@@H]([C@@H](CC(=O)N1CCC[C@H]1[C@H](OC)[C@@H](C)C(=O)N[C@@H](Cc1ccccc1)C(=O)OCc1ccc(OC)cc1OC)OC)N(C)C(=O)[C@@H](NC(=O)[C@H](C(C)C)N(C)C(=O)OCC1c2ccccc2-c2ccccc21)C(C)C]

) without using column chromatography.
Stage 6, Step 2:

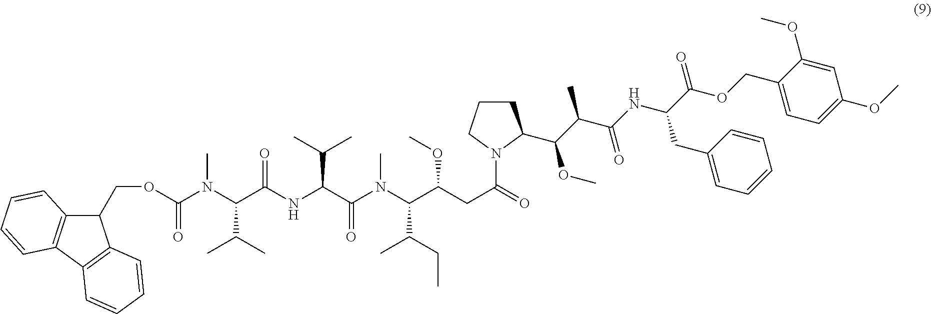 [SMILES: CCC(C)[C@@H]([C@@H](CC(=O)N1CCC[C@H]1[C@H](OC)[C@@H](C)C(=O)N[C@@H](Cc1ccccc1)C(=O)OCc1ccc(OC)cc1OC)OC)N(C)C(=O)[C@@H](NC(=O)[C@H](C(C)C)N(C)C(=O)OCC1c2ccccc2-c2ccccc21)C(C)C]

was converted to

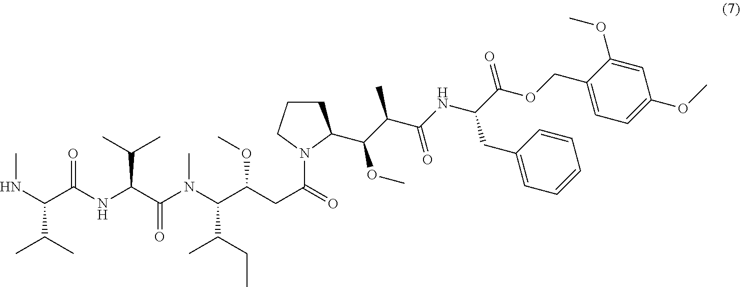 [SMILES: CCC(C)[C@@H]([C@@H](CC(=O)N1CCC[C@H]1[C@H](OC)[C@@H](C)C(=O)N[C@@H](Cc1ccccc1)C(=O)OCc1ccc(OC)cc1OC)OC)N(C)C(=O)[C@@H](NC(=O)[C@@H](NC)C(C)C)C(C)C]

using catalytic amounts of DBU, and

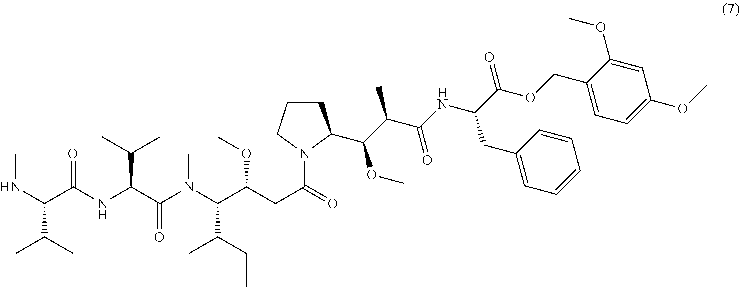 [SMILES: CCC(C)[C@@H]([C@@H](CC(=O)N1CCC[C@H]1[C@H](OC)[C@@H](C)C(=O)N[C@@H](Cc1ccccc1)C(=O)OCc1ccc(OC)cc1OC)OC)N(C)C(=O)[C@@H](NC(=O)[C@@H](NC)C(C)C)C(C)C]

was obtained without using column chromatography.
Stage 6, Step 3:

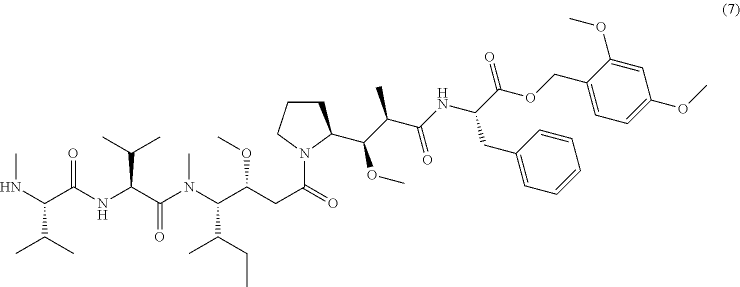 [SMILES: CCC(C)[C@@H]([C@@H](CC(=O)N1CCC[C@H]1[C@H](OC)[C@@H](C)C(=O)N[C@@H](Cc1ccccc1)C(=O)OCc1ccc(OC)cc1OC)OC)N(C)C(=O)[C@@H](NC(=O)[C@@H](NC)C(C)C)C(C)C]

was reacted with

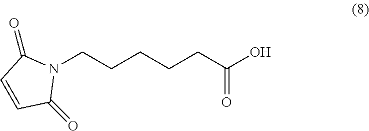 [SMILES: O=C(O)CCCCCN1C(=O)C=CC1=O]

to obtain

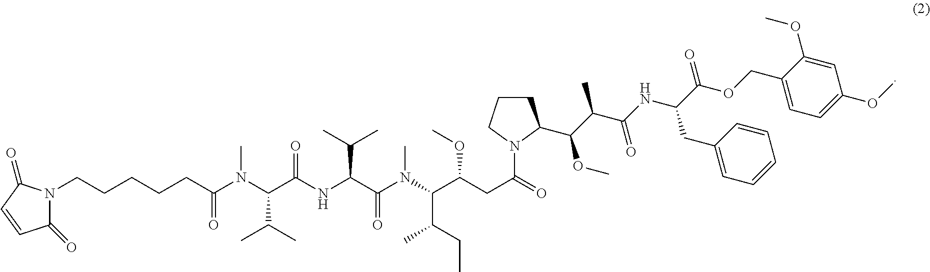

Stage 6, Step 4:

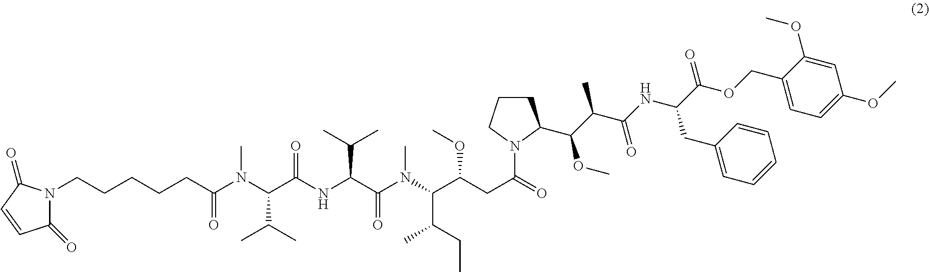 [SMILES: CC[C@H](C)[C@@H]([C@@H](CC(=O)N1CCC[C@H]1[C@H](OC)[C@@H](C)C(=O)N[C@@H](Cc1ccccc1)C(=O)OCc1ccc(OC)cc1OC)OC)N(C)C(=O)[C@@H](NC(=O)[C@H](C(C)C)N(C)C(=O)CCCCCN1C(=O)C=CC1=O)C(C)C]

was converted to mcMMAF in the presence of toluene/2-MeTHF, followed by aqueous workup and HPLC purification.

Some key changes that were made in the process compared to the 1G process have been highlighted in the process map (FIG. 6). In Stage 1, Step 1, the original organic solvent ethyl acetate was replaced with green Surfactant TPGS-750-M. In Stage 1, Step 2, solvent waste was reduced and THF was replaced with 2-MeTHF. In Stage 2, Step 2, HCl/dioxane was replaced with aqueous sulfuric acid and the workup was eliminated, isolating

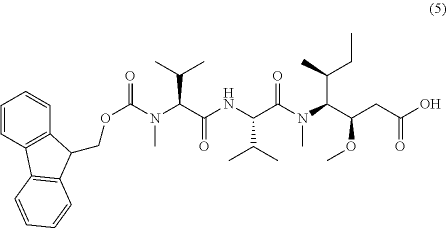 [SMILES: CC[C@H](C)[C@@H]([C@@H](CC(=O)O)OC)N(C)C(=O)[C@@H](NC(=O)[C@H](C(C)C)N(C)C(=O)OCC1c2ccccc2-c2ccccc21)C(C)C]

directly from the reaction.

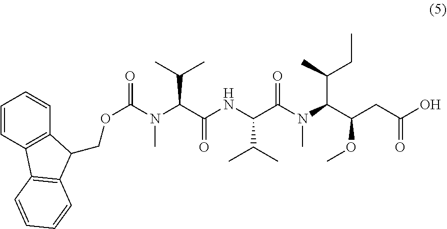 [SMILES: CC[C@H](C)[C@@H]([C@@H](CC(=O)O)OC)N(C)C(=O)[C@@H](NC(=O)[C@H](C(C)C)N(C)C(=O)OCC1c2ccccc2-c2ccccc21)C(C)C]

was isolated as the free acid instead of as the dicyclohexylamine salt, avoiding effective purging of the impurities at this stage and improving manufacturing efficiency by removing the need for chromatographic purification of

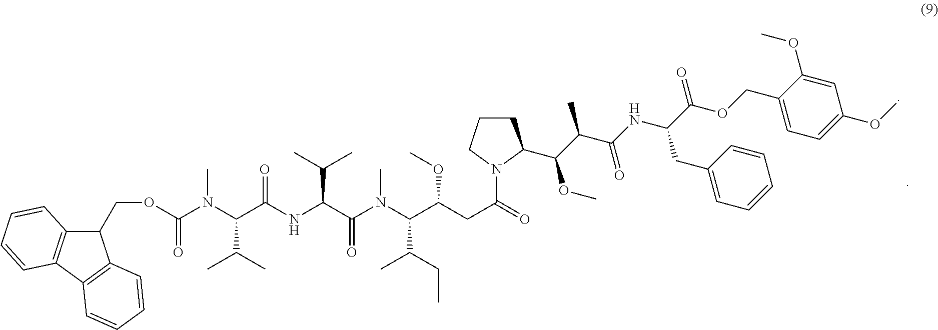

In Stage 3, Step 1, dichloromethane was removed as solvent and

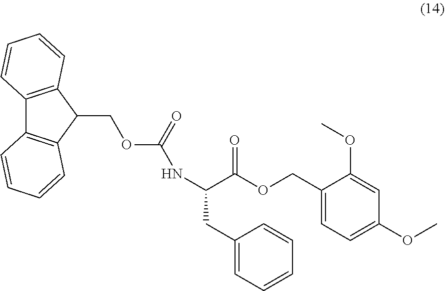 [SMILES: COc1ccc(COC(=O)[C@H](Cc2ccccc2)NC(=O)OCC2c3ccccc3-c3ccccc32)c(OC)c1]

was not isolated, improving manufacturing efficiency relative to the original process by delaying isolation of a solid intermediate until the subsequent

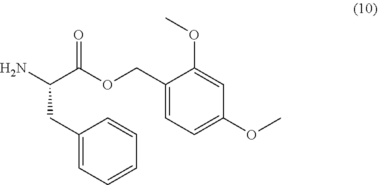 [SMILES: COc1ccc(COC(=O)[C@@H](N)Cc2ccccc2)c(OC)c1]

salt. In Stage 3, Step 2, stoichiometric diethylamine was replaced with a catalytic amount of DBU

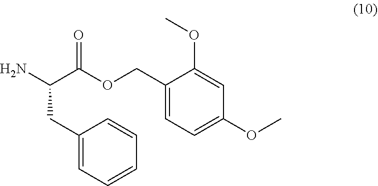 [SMILES: COc1ccc(COC(=O)[C@@H](N)Cc2ccccc2)c(OC)c1]

was isolated as a salt. Isolation of

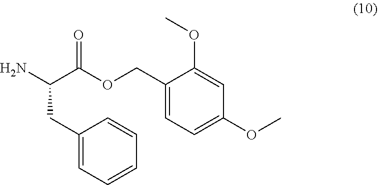 [SMILES: COc1ccc(COC(=O)[C@@H](N)Cc2ccccc2)c(OC)c1]

as a salt affords effective purging of impurities at this stage and improves manufacturing efficiency by removing the need for isolation of

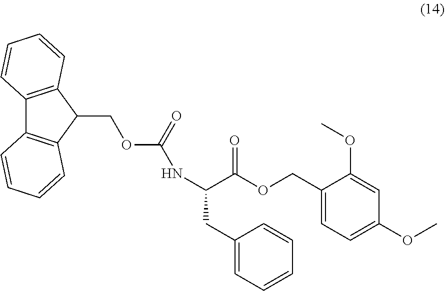 [SMILES: COc1ccc(COC(=O)[C@H](Cc2ccccc2)NC(=O)OCC2c3ccccc3-c3ccccc32)c(OC)c1]

as well as downstream chromatographic purification of

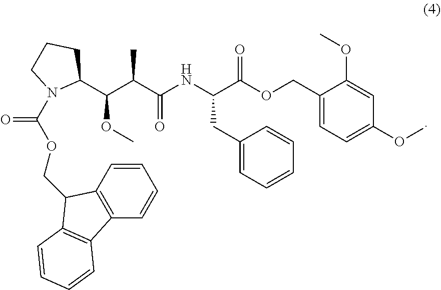

In Stage 4, Step 2,

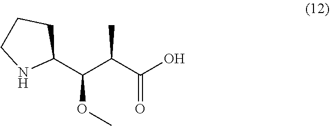

was isolated as the HCl salt and reduced solvent was used. In Stage 5, Step 1, the original solvent dioxane was replaced with a mixture of DMF/water and column chromatography was eliminated. Improved control of reaction by-products provides improved manufacturing efficiency by eliminating the need for chromatographic purification of

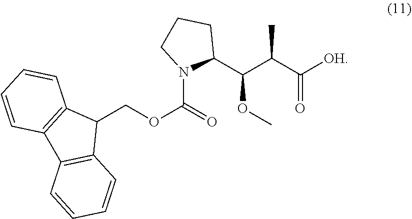 [SMILES: CO[C@H]([C@@H](C)C(=O)O)[C@@H]1CCCN1C(=O)OCC1c2ccccc2-c2ccccc21]

In Stage 5, Step 2, column chromatography was eliminated. Improved control of impurities in earlier stages provides improved manufacturing efficiency by eliminating the need for chromatographic purification of

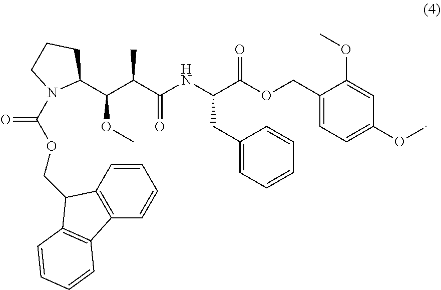

In Stage 5, Step 3, stoichiometric diethylamine was replaced with a catalytic amount of DBU

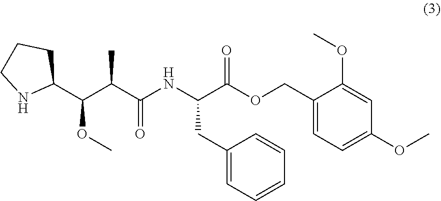 [SMILES: COc1ccc(COC(=O)[C@H](Cc2ccccc2)NC(=O)[C@H](C)[C@@H](OC)[C@@H]2CCCN2)c(OC)c1]

was isolated as a salt. Isolation of

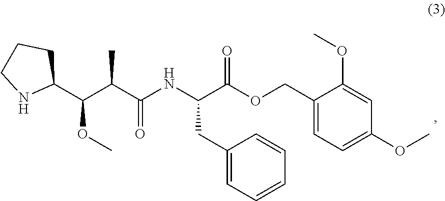 [SMILES: COc1ccc(COC(=O)[C@H](Cc2ccccc2)NC(=O)[C@H](C)[C@@H](OC)[C@@H]2CCCN2)c(OC)c1]

as a salt affords effective purging of impurities at this stage and improves manufacturing efficiency by sequestering processing of all high-potency intermediates to Stage 6 and eliminating the need for downstream chromatographic purification of

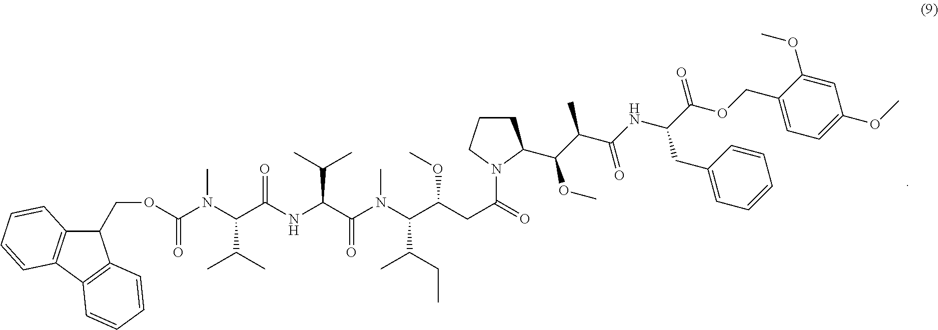 [SMILES: CCC(C)[C@@H]([C@@H](CC(=O)N1CCC[C@H]1[C@H](OC)[C@@H](C)C(=O)N[C@@H](Cc1ccccc1)C(=O)OCc1ccc(OC)cc1OC)OC)N(C)C(=O)[C@@H](NC(=O)[C@H](C(C)C)N(C)C(=O)OCC1c2ccccc2-c2ccccc21)C(C)C]

In Stage 6, Step 1 column chromatography was eliminated. Isolation of

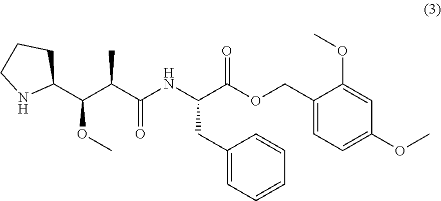 [SMILES: COc1ccc(COC(=O)[C@H](Cc2ccccc2)NC(=O)[C@H](C)[C@@H](OC)[C@@H]2CCCN2)c(OC)c1]

as a salt in the preceding stage and improved impurity control in earlier manufacturing stages has improved manufacturing efficiency by eliminating the need for chromatographic purification of

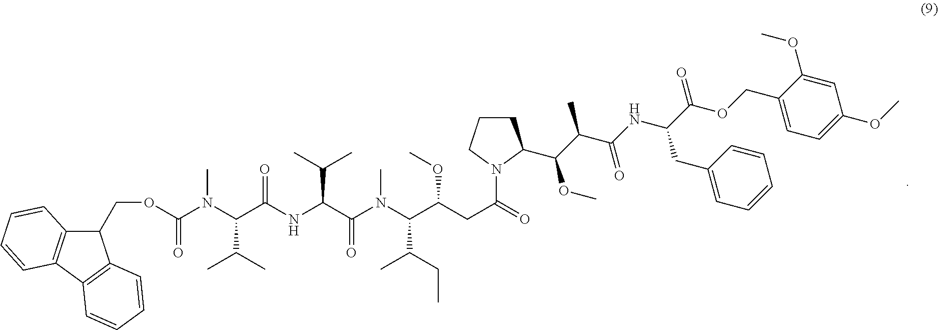 [SMILES: CCC(C)[C@@H]([C@@H](CC(=O)N1CCC[C@H]1[C@H](OC)[C@@H](C)C(=O)N[C@@H](Cc1ccccc1)C(=O)OCc1ccc(OC)cc1OC)OC)N(C)C(=O)[C@@H](NC(=O)[C@H](C(C)C)N(C)C(=O)OCC1c2ccccc2-c2ccccc21)C(C)C]

In Stage 6, Step 2, stoichiometric diethylamine was replaced with a catalytic amount of DBU and column chromatography was eliminated. Improved control of reaction by-products has improved manufacturing efficiency by eliminating the need for chromatographic purification of

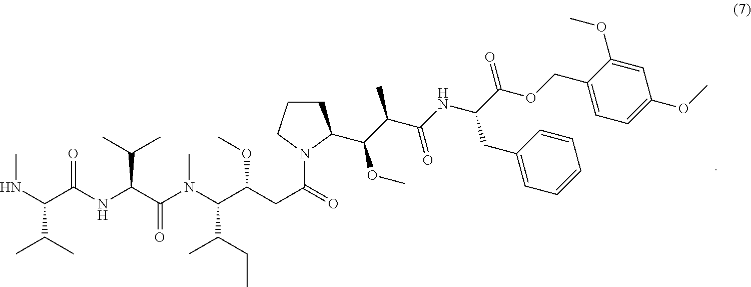 [SMILES: CCC(C)[C@@H]([C@@H](CC(=O)N1CCC[C@H]1[C@H](OC)[C@@H](C)C(=O)N[C@@H](Cc1ccccc1)C(=O)OCc1ccc(OC)cc1OC)OC)N(C)C(=O)[C@@H](NC(=O)[C@@H](NC)C(C)C)C(C)C]

In Stage 6, Step 4, DCM as reaction solvent was replaced with toluene/2-MeTHF, and expensive, hazardous removal of excess trifluoroacetic acid via evaporation was replaced by an aqueous workup. A final chromatographic separation continues to have a role to control the purity and quality attributes for mcMMAF as this peptide is not as crystalline and thus it is difficult to isolate it directly from the reaction through standard crystallization processes. The result was the successful replacement of the single use flash chromatography by a more efficient high pressure chromatographic purification system. This process change has reduced solvent consumption (vide infra) and eliminated the need for a new purification stationary phase (cartridge) for every 300 g of crude mcMMAF produced.

Stages 1-5 were validated, and the validation campaign summary is shown in Table 2.

TABLE 2
Validation Campaign Summary.

| | | Average | | Average | |
|---|---|---|---|---|---|
| Process | Number of | Yield | | Offload | |
| Stage | Batches | (% th) | Input Scale | Weight | |
| Stage 1 | 6 | 81% | 1.5 kg based on starting | 2.17 | kg |
| | | | material (18)-HCl | | |
| Stage 2 | 4 | 90% | 2.3 kg based on starting | 2.5 | kg |
| | | | material (16)-PTSA | | |
| Stage 3 | 6 | 75% | 1.8 kg based on starting | 1.23 | kg |
| | | | material (15) | | |
| Stage 4 | 8 | 86% | 1.8 kg based on starting | 0.74 | kg |
| | | | material (13)-DCHA | | |
| Stage 5 | 6 | 76% | 0.5 kg based on starting | 0.89 | kg |
| | | | material (12)-HCl | | |

Stage 6 was demonstrated at intended commercial scale (Table 3).

TABLE 3
Engineering Campaign Summary.

| | | Average | | Average |
|---|---|---|---|---|
| Process | Number of | Yield | | Offload |
| Stage | Batches | (% th) | Input Scale | Weight |
| Stage 6 | 2 | 56% | 0.6 kg based on starting | 0.49 kg |
| | | | material (5) | |

Process metrics for 2G process calculated using ACSGCI PMI-LCA Tool are shown in Table 4.

TABLE 4
Process Metrics for 1G Process Calculated using ACSGCI PMI-LCA Tool.

| Process Metrics per kg API | Total | Reagent | Metal | Solvent | Water |
|---|---|---|---|---|---|
| PMI | 2753.53 | 41.31 | 0.27 | 2376.23 | 335.72 |
| Mass Net (kg) | 6010.91 | 1189.64 | 232.22 | 4588.54 | 0.51 |
| Energy (MJ) | 158533.08 | 23471.88 | 4862.47 | 130192.34 | 6.40 |
| GWP (kg CO2 equiv.) | 9578.50 | 2379.42 | 260.12 | 6938.63 | 0.34 |
| Acidification (kg SO2 equiv.) | 249.04 | 19.74 | 189.45 | 39.84 | 0.00 |
| Eutrophication (kg phosphate equiv.) | 11.31 | 2.33 | 1.87 | 7.11 | 0.00 |
| Water (kg) | 29947.16 | 401.67 | 8247.63 | 20873.72 | 424.14 |

Comparison of Process Metrics for mcMMAF 1G Versus 2G Process

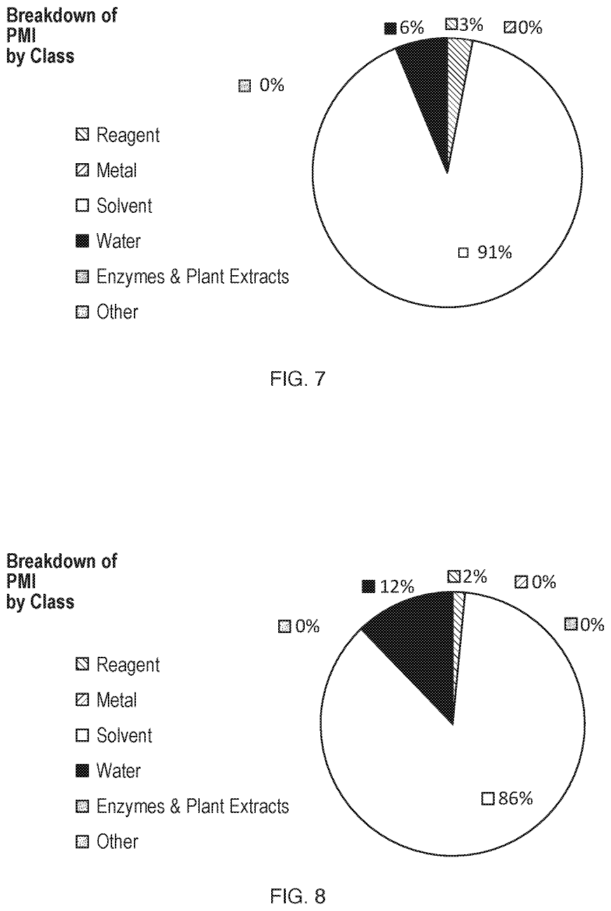

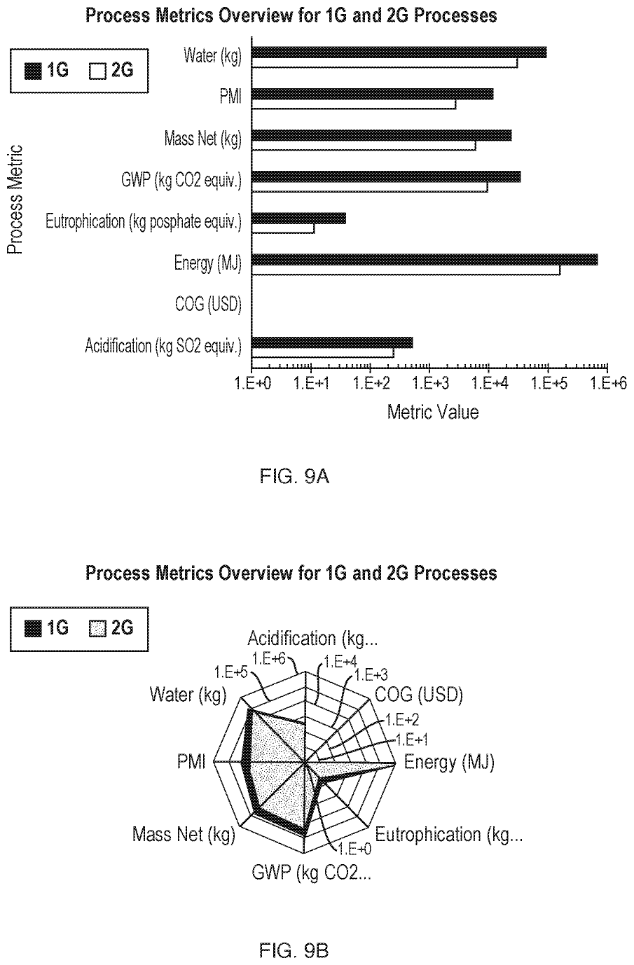

A 76% reduction of PMI was achieved using the 2G process for the manufacturing of mcMMAF. In addition, a comparison of the breakdowns of PMIs by class shows that there is a reduction in contribution of solvents (−5%) and reagents (−1%) between the 1G and 2G processes. In addition, this reduction in consumption of organic solvents is balanced by an increase in contribution of water (+6%) for the 2G process (FIG. 7 and FIG. 8). This change from solvent to water use reduces the environmental impact of the process. Solvents will have a typical carbon footprint of 1-3 kg CO₂ eq. per kg whereas water will have a carbon footprint close to zero (Cespi, D., et al. Green Chem., 2015, 17, 3390-3400, herein incorporated by reference in its entirety).

Overall, there is a 71% reduction of GWP between the 2G and 1G process, equivalent to 24000 Kg of CO₂ removed with this new process (Table 5). Thus, the 2 G process represents a significant reduction of emissions. Another important reduction can be observed in calculated energy consumption. Again, the 2G processes has a 76% reduction in energy relative to the 1G Process, which translates to a reduction of more than 507000 MJ or 14000 kWh of energy saved per Kg of mcMMAF. Both acidification (propensity of the process to release acid into the environment) and eutrophication (process potential to add nutrients) into the natural environment were also significantly reduced (51 and 70% respectively). Finally, the 2G process represents about 62000 Kg of water saved per kg of mcMMAF.

TABLE 5
Comparison of Process Metrics for 1G vs. 2G
Process Calculated using ACSGCI PMI-LCA Tool.

| Process Metrics per kg API | 1G Process | 2G Process | Reduction | Percent Reduction |
|---|---|---|---|---|
| PMI | 11581.04 | 2753.53 | 8827.51 | 76% |
| Mass Net (kg) | 23680.37 | 6010.91 | 17669.46 | 75% |
| Energy (MJ) | 665325.86 | 158533.08 | 506792.78 | 76% |
| GWP (kg CO2 equiv.) | 33457.66 | 9578.5 | 23879.16 | 71% |
| Acidification (kg SO2 equiv.) | 511.7 | 249.04 | 262.66 | 51% |
| Eutrophication (kg phosphate equiv.) | 37.94 | 11.31 | 26.63 | 70% |
| Water (kg) | 92489.01 | 29947.16 | 62541.85 | 68% |

A second generation (2G) process has been developed using the original synthetic route for mcMMAF (1) that is more efficient and sustainable than the first-generation process. As calculated using the ACSCGI PMI_LCA tool, the 2G process demonstrates a pivotal reduction of all metrics and in some cases even in order of magnitudes. Significant reductions in PMI (−8827), emissions (−23879 kg CO₂) and energy usage (−51000 MJ) were achieved using this process. In addition, a reduction with potential acidification and eutrophication of the environment was also achieved.

### Example 2

Abbreviations used herein are provided in Table 6.

TABLE 6
Abbreviations.

| | |
|---|---|
| Boc | tert-butyloxycarbonyl |
| tBu | tert-butyl |
| Cbz or Z | benzyloxycarbonyl |
| CMPI | 2-chloro-1-methylpyridinium iodide |
| DBU | 1,8-diazabicyclo[5.4.0]undec-7-ene |
| DCHA | dicyclohexylamine |
| DCM | dichloromethane |
| DIC | N,N′-diisopropylcarbodiimide |
| DIPEA | N,N-diisopropylethylamine |
| DMAP | 4-dimethylaminopyridine |
| DMF | N,N-dimethylformamide |
| DMTMM | 4-(4,6-dimethoxy-1,3,5-triazin-2-yl)-4- |
| | methylmorpholinium chloride |
| EtOAc | ethyl acetate |
| Fmoc | 9H-fluorenylmethoxycarbonyl |
| Fmoc-Osu | N-(9H-fluorenylmethoxycarbonyloxy)succinimide |
| IPAc | isopropyl acetate |
| Me | methyl |
| MeCN | acetonitrile |
| 2-MeTHF | 2-methyltetrahydrofuran |
| NMM | N-methylmorpholine |
| Ph | phenyl |
| PhMe | toluene |
| 2-PrOH | 2-propanol |
| Pd/C | 10% wt Palladium on Carbon 50% Wet |
| PTSA•H₂O | p-toluenesulfonic acid monohydrate |
| TBME | tert-butyl methyl ether |
| TBTU | O-(50aleimidocapr-1-yl)-N,N,N′,N′-tetramethyluronium |
| | tetrafluoroborate |
| TFA | trifluoroacetic acid |
| THF | tetrahydrofuran |
| TPGS-750-M | DL-α-tocopherol methoxypolyethylene glycol succinate, |
| | 2% wt water solution |

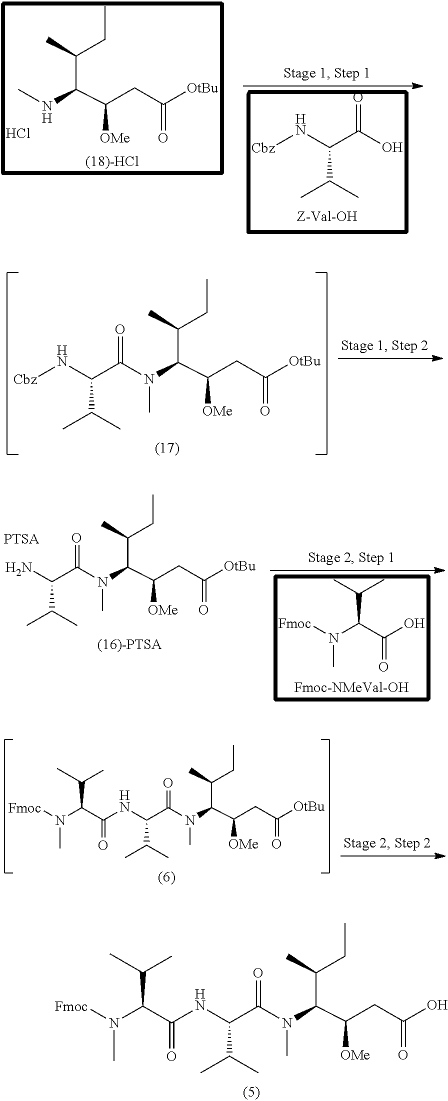

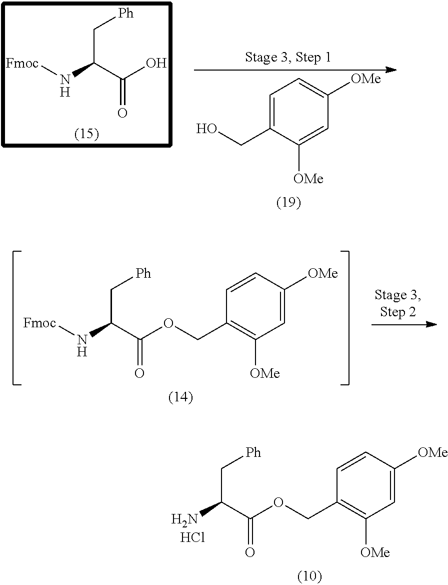

### Key:

Stage 1, Step 1: DMTMM, NMM, TPGS-750-M/H₂O, IPAc. Stage 2, Step 1: TBTU, DIPEA, EtOAc.
Stage 1, Step 2: Pd/C, H₂, PTSA·H₂O, 2-MeTHF, IPAc Stage 2, Step 2: H₂SO₄, MeCN.
Stage 3, Step 1: DIC, DMAP, EtOAc.
Stage 3, Step 2: 1-octanethiol, DBU, HCl/1,4-dioxane, EtOAc

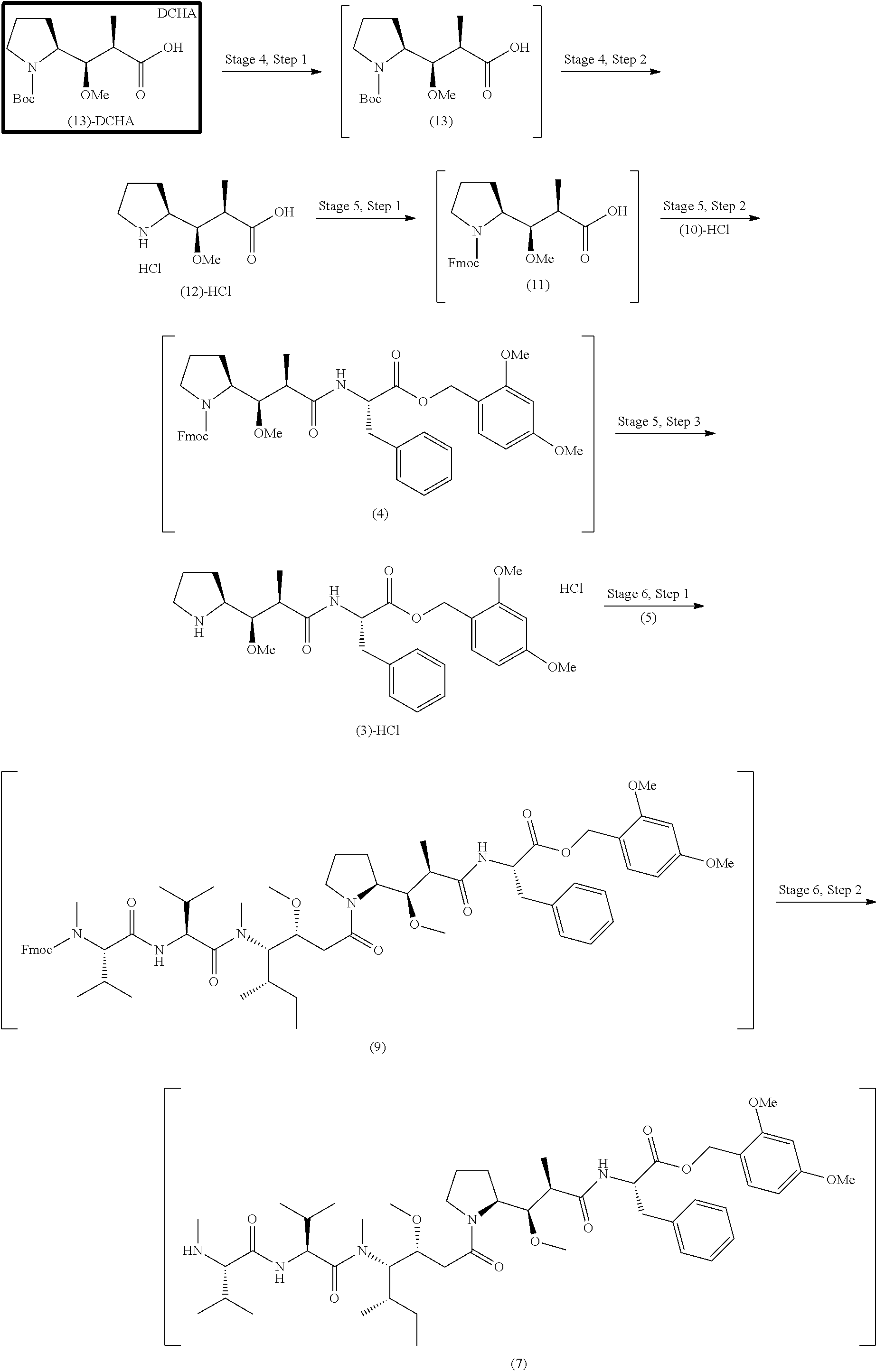

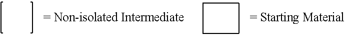

### Key:

Stage 4, Step 1: H₂SO₄, TBME. Stage 6, Step 1: TBTU, DIPEA, EtOAc.
Stage 4, Step 2: HCl/1,4-dioxane, heptane. Stage 6, Step 2: DBU, MeCN
Stage 5, Step 1: Fmoc-OSu, NaHCO₃, DMF/H₂O, heptane.
Stage 5, Step 2: TBTU, DIPEA, EtOAc
Stage 5, Step 3: DBU, 1-octanethiol, EtOAc, HCl/1,4-dioxane

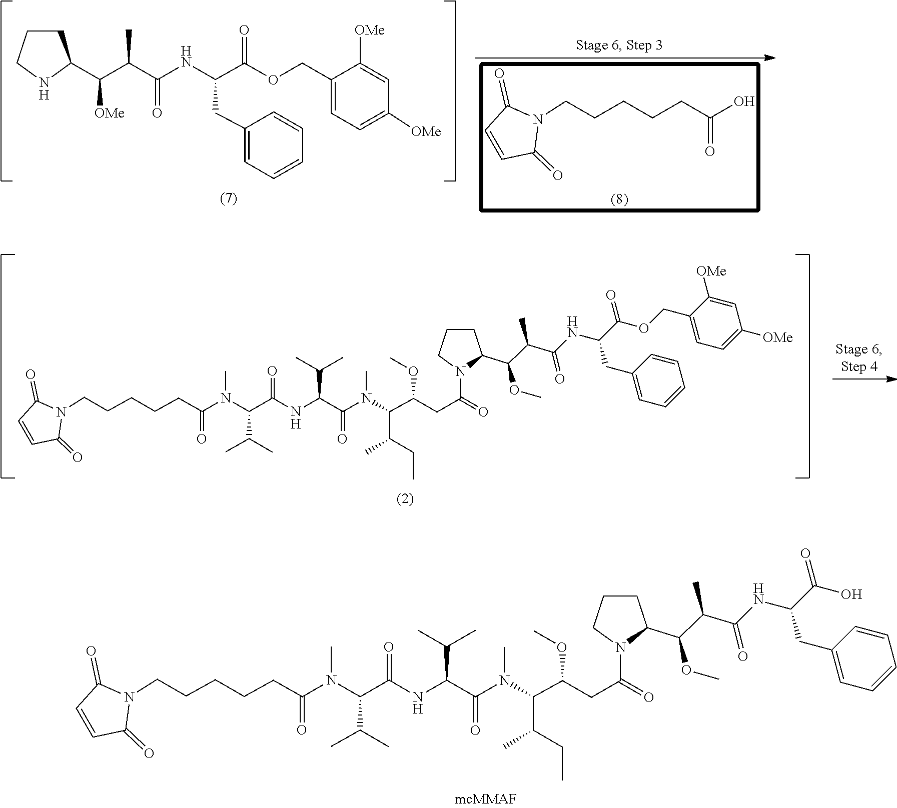

### Key:

Stage 6, Step 3: CMPI, DIPEA, PhMe.
Stage 6, Step 4: TFA, PhMe, 2-MeTHF,
   chromatographic purification in DCM, 2-PrOH

Scheme 2 illustrates the synthetic flow chart for the manufacture of Maleimidocaproyl Monomethyl Auristatin F (mcMMAF), as shown in FIG. 6 of the 2G process map.

### Example 2.1: Preparation of (16)-PTSA

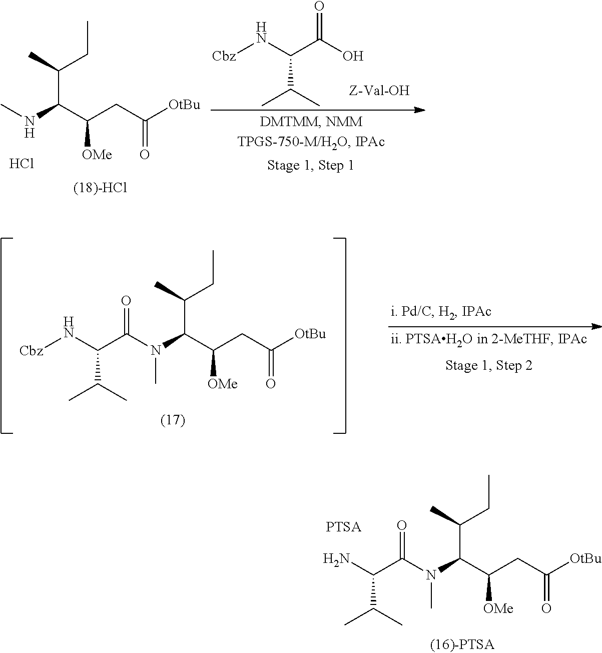

Stage 1, Step 1: (3R,4S,5S)-1-(tert-butoxy)-3-methoxy-N,5-dimethyl-1-oxoheptan-4-ammonium chloride ((18)-HCl)) (1.0 molar equivalent) was reacted with Z-Val-OH (1.08-1.12 molar equivalents), NMM (1.96-2.04 molar equivalents) and DMTMM (1.47-1.53 molar equivalents) in aqueous TPGS-750-M (3.5-4.5 volumes) and isopropyl acetate (2-4 volumes) at 15-25° C. for not less than 9 hours. The reaction mixture was diluted with isopropyl acetate and washed with aqueous tribasic potassium phosphate and aqueous NaCl/HCl to give a solution of (3R,4S,5S)-tert-butyl 4-[(S)-2-{[(benzyloxy)carbonyl]amino}-N,3-dimethylbutanamido]-3-methoxy-5-methylheptanoate (17).

Stage 1, Step 2: The solution of (17) was hydrogenated in the presence of Pd/C (not less than 26.5% w/w) at 15-25° C., at a pressure of not less than 3.2 bar for a total time of not less than 3 hours, filtered and distilled from isopropyl acetate. Isopropyl acetate, a solution of PTSA monohydrate (0.95-1.05 molar equivalents) in 2-MeTHF and (S)-1-{[(3R,4S,5S)-1-(tert-butoxy)-3-methoxy-5-methyl-1-oxoheptan-4-yl](methyl)amino}-3-methyl-1-oxobutan-2-ammonium 4-methylbenzenesulfonate ((16)-PTSA) seed were added. The reaction was distilled from isopropyl acetate. The mixture was cooled to 0-10° C., filtered, washed with isopropyl acetate (not less than 15 volumes) and dried. Typical yield was 61-85% of theory.

¹H NMR (500 MHz, DMSO-d6): δ 7.93 (bro s, 3H), 7.48 (d, 2H), 7.11 (2H), 4.63 (br s, 1H), 4.24 (br d, 1H), 3.79 (br s, 1H), 3.24 (s, 1H), 2.86 (br s, 3H), 2.61 (dd, 1H), 2.29 (s, 3H), 2.17-2.13 (m, 2H), 1.79 (m, 1H), 1.41 (m, 9H), 1.36 (m, 1H), 1.04 (d, 3H), 0.95 (m, 1H), 0.92-0.91 (m, 7H), 0.84 (t, 3H). LC/MS ESI+ [M+H]⁺ Calculated for C₁₉H₃₉N₂O₄ 359.3, found: 359.3.

### Example 2.2: Preparation of (5)

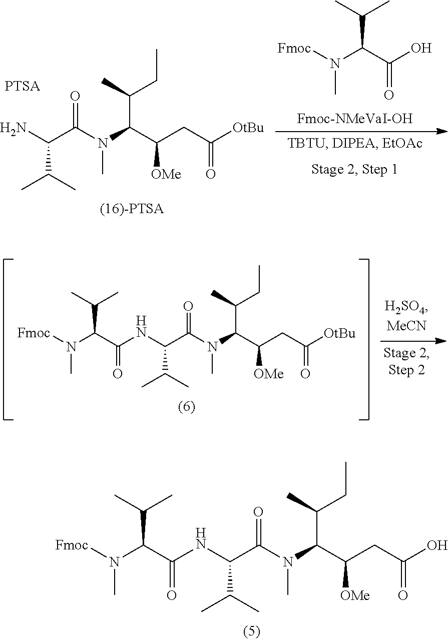

Stage 2, Step 1: (16)-PTSA (1.0 molar equivalent) was reacted with Fmoc-NmeVal-OH (0.98-1.04 molar equivalents), DIPEA (2.95-3.05 molar equivalents) and TBTU (1.03-1.08 molar equivalents) in ethyl acetate (7-9 volumes) at 15-25° C. for not less than 6 hours. The reaction mixture was washed with water and distilled, replacing ethyl acetate with acetonitrile to give a solution of (5S,8S,11S,12R)-tert-butyl 11-[(S)-sec-butyl]-1-(9H-fluoren-9-yl)-5,8-diisopropyl-12-methoxy-4,10-dimethyl-3,6,9-trioxo-2-oxa-4,7,10-triazatetradecan-14-oate (6).

Stage 2, Step 2: The solution of (6) was diluted with acetonitrile and water and reacted with sulfuric acid (2.95-3.05 molar equivalents) at 5-15° C. for not less than 12 hours. (5S,8S,11S,12R)-11-((S)-sec-butyl)-1-(9H-fluoren-9-yl)-5,8-diisopropyl-12-methoxy-4,10-dimethyl-3,6,9-trioxo-2-oxa-4,7,10-triazatetradecan-14-oic acid (5) seed and water were added. The mixture was cooled to −5-5° C. and (5) was isolated by filtration, washed with acetonitrile in water (not less than 20 volumes) and dried. Typical yield was 80-95% of theory.

¹H NMR (500 MHz, DMSO-d6): δ 9.60 (br d, 1H), 7.89 (br d, 2H), 7.72 (br dd, 2H), 7.45 (br t, 2H), 7.37 (m, 2H), 5.22 (br s, 1H), 5.01 (br t, 1H), 4.88 (br d, 1H), 4.62 (m, 2H), 4.39-4.38 (m, 2H), 3.58 (s, 3H), 3.28 (s, 3H), 3.27 (s, 3H), 2.98 (m, 1H), 2.85 (dd, 1H), 2.41-2.34 (m, 2H), 1.88 (br m, 1H), 1.62 (br m, 1H), 1.21 (m, 1H), 1.10-0.85 (m, 18H). LC/MS ESI+ [M+H]⁺ Calculated for C₃₆H₅₂N₃O₇ 638.4, found: 638.4.

Volumes and molar equivalents were expressed relative to the quantity of (16)-PTSA.

### Example 2.3: Preparation of (10)-HCl

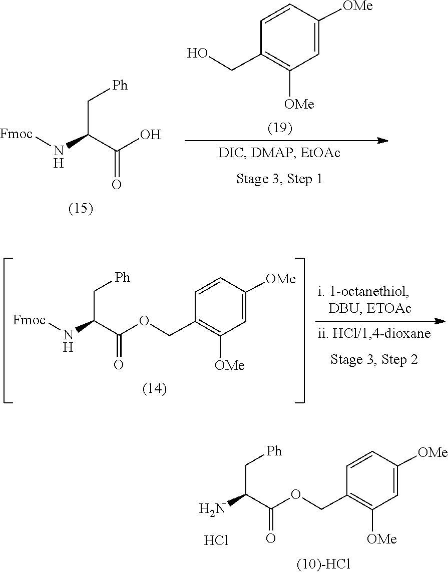

Stage 3, Step 1: (S)-2-({[(9H-fluoren-9-yl)methoxy]carbonyl}-amino)-3-phenylpropanoic acid (15) (1.0 molar equivalent) was reacted with 2,4-dimethoxybenzyl alcohol (19) (1.08-1.12 molar equivalents) in the presence of DMAP (0.048-0.053 molar equivalents), DIC (1.08-1.12 molar equivalents) and ethyl acetate (14-16 volumes) at −6-6° C. for not less than 20 hours. The reaction mixture was washed with sodium chloride in aqueous phosphoric acid followed by aqueous potassium phosphate dibasic and distilled from ethyl acetate to give a solution of (S)-2,4-dimethoxybenzyl 2-({[(9H-fluoren-9-yl)methoxy]carbonyl}amino)-3-phenylpropanoate (14).

Stage 3, Step 2: Ethyl acetate, 1-octanethiol (1.47-1.53 molar equivalents) and DBU (0.095-0.105 molar equivalents) were added to the solution of (14). The resulting mixture was reacted at 25-36° C. for 8-24 hours. The reaction mixture was washed with water and diluted with ethyl acetate. Hydrochloric acid in 1,4-dioxane (0.98-1.02 molar equivalents) was added to crystallize 2,4-dimethoxybenzyl L-phenylalaninate hydrochloride ((10)-HCl)) which was isolated by filtration, washed with ethyl acetate (not less than 15 volumes) and dried. Typical yield was 65-90% of theory.

¹H NMR (700 MHz, DMSO-d6): δ 8.62 (br s, 3H), 7.28-7.27 (m, 3H), 7.18 (m, 2H), 7.13 (d, 1H), 6.60 (d, 1H), 6.50 (dd, 1H), 5.06 (s, 2H), 4.28 (dd, 1H), 3.79 (s, 3H), 3.78 (s, 3H), 3.15 (dd, 1H), 3.07 (dd, 1H). LC/MS ESI+ [M+H]⁺ Calculated for C₁₈H₂₂NO₄ 316.2, found: 316.2.

Volumes and molar equivalents were expressed relative to the quantity of (15).

### Example 2.4: Preparation of (12)-HCl

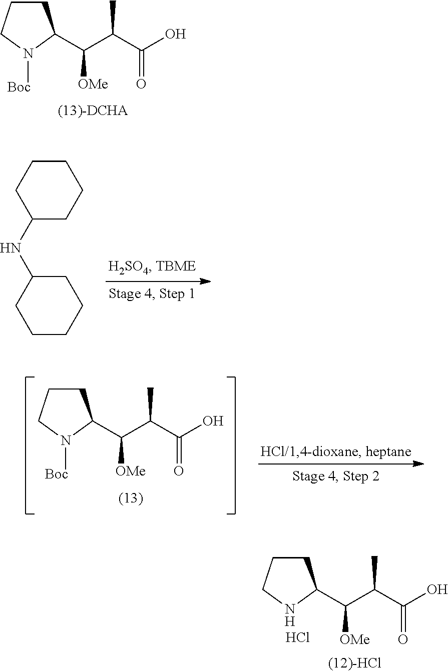

Stage 4, Step 1: (2R,3R)-3-[(S)-1-(tert-butoxycarbonyl)57aleimidoca-2-yl]-3-methoxy-2-methylpropanoic acid ((13)-DCHA)) (1.0 molar equivalent) in TBME was washed with aqueous sulfuric acid then water. The mixture was distilled with 1,4-dioxane to replace TBME.

Stage 4, Step 2: HCl in 1,4-dioxane (4.1-5.4 molar equivalents) was added to the solution of (13) and the resulting mixture was held at 15-25° C. for 8-16 hours. (2R,3R)-3-methoxy-2-methyl-3-((S)-pyrrolidin-2-yl)propanoic acid hydrochloride ((12)-HCl)) seed and heptane were added and the resulting mixture was held for not less than 2.5 hours. (12)-HCl was obtained by filtration, washing with heptane and drying. Typical yield was 65-99% of theory.

¹H NMR (500 MHz, DMSO-d6): δ 12.56 (br s, 1H), 9.93 (br s, 1H), 8.70 (br s, 1H), 3.89 (t, 1H), 3.49 (m, 1H), 3.39 (s, 3H), 3.13 (m, 2H), 2.66 (m, 1H), 1.99 (m, 1H), 1.92 (m, 1H), 1.82 (m, 1H), 1.77 (m, 1H), 1.11 (d, 3H). LC/MS ESI+ [M+H]⁺ Calculated for C₉H₁₈NO₃ 188.1, found: 188.1.

Volumes and molar equivalents were expressed relative to the quantity of (13)-DCHA.

### Example 2.5: Preparation of (3)-HCl

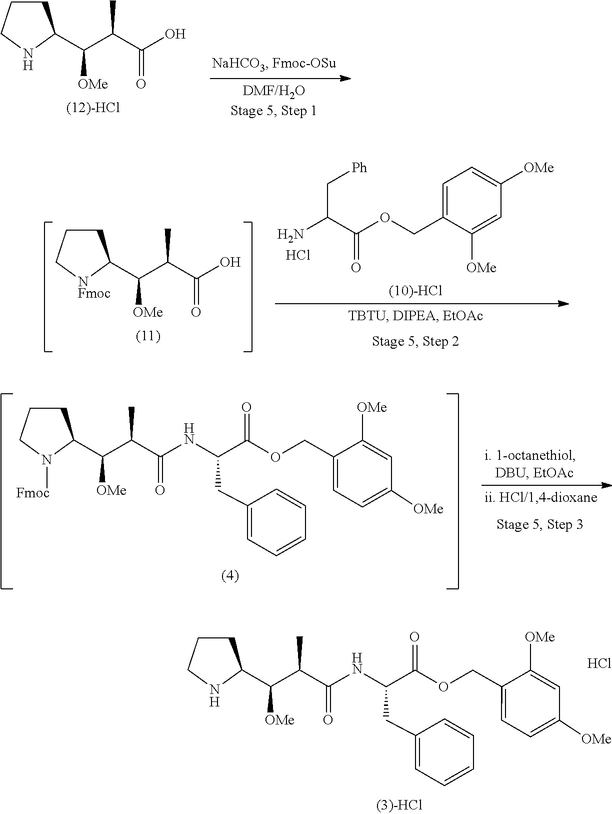

Stage 5, Step 1: (12)-HCl (1.0 molar equivalent) was reacted with Fmoc-Osu (1.05-1.20 equivalents) and sodium bicarbonate (2.1-2.3 equivalents) in DMF-water (9-11 volumes) at 30-40° C. for not less than 9 hours. heptane was added and the phases are separated. Ethyl acetate, water and phosphoric acid were charged to the aqueous layer. The phases were separated and the organic layer containing (2R,3R)-3-[(S)-1-{[(9H-fluoren-9-yl)methoxy]carbonyl}58aleimidoca-2-yl]-3-methoxy-2-methylpropanoic acid (11) was washed with water.

Stage 5, Step 2: The solution of (11) was diluted with ethyl acetate (4-6 volumes) and reacted with TBTU (1.05-1.25 equivalents), 2,4-dimethoxybenzyl L-phenylalaninate hydrochloride ((10)-HCl)) (0.98-1.05 equivalents) and DIPEA (2.94-3.15 equivalents) at 10-20° C. for not less than 6 hours. The reaction mixture was washed with water, aqueous hydrochloric acid and aqueous potassium phosphate dibasic then distilled. Additional ethyl acetate was added to give a solution of (S)-(9H-fluoren-9-yl)methyl 2-[(1R,2R)-3-({(S)-1-[(2,4-dimethoxybenzyl)oxy]-1-oxo-3-phenylpropan-2-yl}amino)-1-methoxy-2-methyl-3-oxopropyl]pyrrolidine-1-carboxylate (4).

Stage 5, Step 3: The solution of (4) was treated with 1-octanethiol (1.1-1.3 equivalents) and DBU (0.53-0.60 equivalents) for 6-8 hours at 3-9° C., washed with water at 3-9° C. and diluted with ethyl acetate. The mixture was distilled to 4.5-7.5 volumes for a total time of not more than 13 hours. If the distillation temperature exceeds 20° C., a temperature of up to 25° C. for a total time of up to 60 minutes within the total time of 13 hours was permitted. The mixture was diluted with ethyl acetate (10.5-13.5 volumes). At −5-5° C. hydrochloric acid in 1,4-dioxane (0.10-0.20 molar equivalents) and 2,4-dimethoxybenzyl ((2R,3R)-3-methoxy-2-methyl-3-((S)-pyrrolidin-2-yl)propanoyl)-L-phenylalaninate hydrochloride ((3)-HCl)) seed (not less than 0.5% w/w) were added. Additional hydrochloric acid in 1,4-dioxane (0.65-0.80 molar equivalents) was then added over not less than 30 minutes to crystallize (3)-HCl. The product was isolated by filtration, washed with ethyl acetate and dried at not more than 60° C. Typical yield was 50-90% of theory.

¹H NMR (700 MHz, DMSO-d6): δ 9.54 (br s, 1H), 8.61 (d, 1H), 8.55 (br s, 1H), 7.28-7.27 (m, 4H), 7.21 (m, 2H), 6.59 (d, 1H), 6.51 (dd, 1H), 5.07 (d, 1H), 5.02 (d, 1H), 4.54 (ddd, 1H), 3.79 (s, 3H), 3.77 (s, 3H), 3.52 (dd, 1H), 3.20 (s, 3H), 3.09-3.01 (m, 4H), 2.87 (dd, 1H), 2.46 (dq, 1H), 1.80 (m, 1H), 1.63-1.61 (m, 2H), 1.49 (m, 1H), 1.03 (d, 3H). LC/MS ESI+ [M+H]⁺ Calculated for C₂₇H₃₇N₂O₆ 485.3, found: 485.3.

Volumes and molar equivalents were expressed relative to the quantity of (12)-HCl.

### Example 2.6: Preparation of mcMMAF

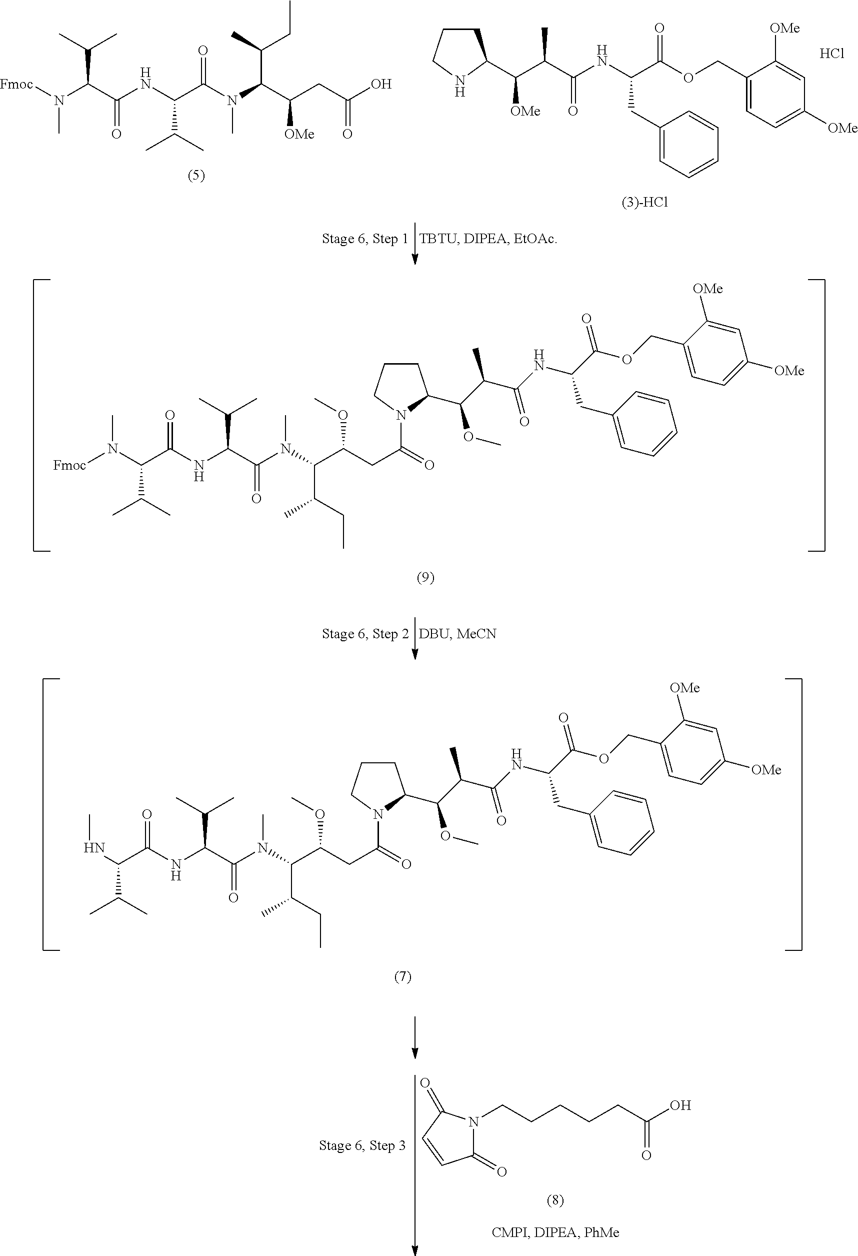

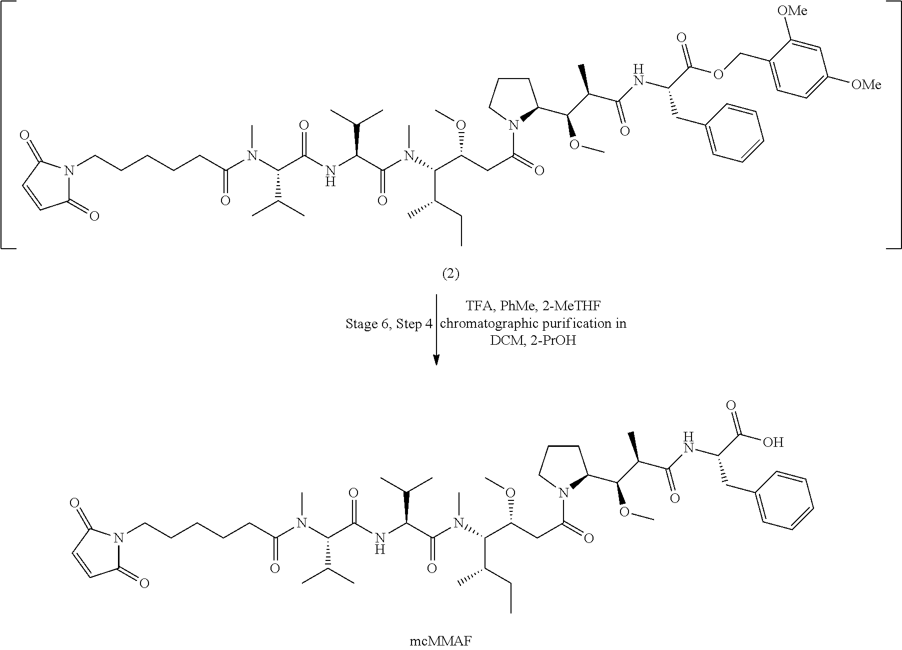

Stage 6, Step 1: (5) (1.0 molar equivalent) was reacted with (3)-HCl (1.05-1.07 molar equivalents), TBTU (1.07-1.13 molar equivalents) and DIPEA (2.30-2.50 molar equivalents) in ethyl acetate (14-18 volumes) at 20-30° C. for not less than 19 hours. The resulting solution of (S)-2,4-dimethoxybenzyl 2-{(2R,3R)-3-[(S)-1-{(5S,8S,11S,12R)-11-[(S)-sec-butyl]-1-(9H-fluoren-9-yl)-5, 8-diisopropyl-12-methoxy-4,10-dimethyl-3,6,9-trioxo-2-oxa-4,7,10-triazatetradecan-14-oyl}pyrrolidin-2-yl]-3-methoxy-2-methylpropanamido}-3-phenylpropanoate (9) was diluted with heptane (5-6 volumes), washed with aqueous sulfuric acid, water and aqueous sodium bicarbonate then distilled to 6-10 volumes, exchanging ethyl acetate with acetonitrile.

Stage 6, Step 2: The solution of (9) in acetonitrile was reacted with DBU (0.05-0.10 molar equivalents) at 20-30° C. until the level of (9) was not more than 2.0% area as determined by in process control, followed by washing with heptane to give a solution of (S)-2,4-dimethoxybenzyl 2-[(2R,3R)-3-{(S)-1-[(3R,4S,5S)-4-{(S)—N,3-dimethyl-2-[(S)-3-methyl-2-(methylamino)butanamido]butanamido}-3-methoxy-5-methylheptanoyl]62aleimidoca-2-yl}-3-methoxy-2-methylpropanamido]-3-phenylpropanoate (7).

Stage 6, Step 3: The solution of (7) was reacted with 6-(2,5-dioxo-2,5-dihydro-1H-pyrrol-1-yl)hexanoic acid (8) (1.05-1.15 molar equivalents) in the presence of CMPI (1.10-1.20 molar equivalents) and DIPEA (2.00-2.20 molar equivalents) at 20-30° C. for not less than 5 hours. The mixture was diluted with toluene, washed with aqueous sulfuric acid and water, distilled to 6.5-10 volumes, exchanging acetonitrile for toluene, then diluted with toluene (to a total volume of 23-33 volumes) to give a solution of (S)-2,4-dimethoxybenzyl 2-{(2R,3R)-3-[(S)-1-{(3R,4S,5S)-4-[(S)-2-{(S)-2-[6-(2,5-dioxo-2,5-dihydro-1H-pyrrol-1-yl)-N-methylhexanamido]-3-methylbutanamido}-N,3-dimethylbutanamido]-3-methoxy-5-methylheptanoyl}62aleimidoca-2-yl]-3-methoxy-2-methylpropanamido}-3-phenylpropanoate (2).

Stage 6, Step 4: The solution of (2) was reacted with trifluoroacetic acid (6.50-6.90 molar equivalents) at −5-5° C. for 4-6 hours. The mixture was filtered with the addition of 2-Me-THF. The filtrate was washed with phosphate buffer, aqueous phosphoric acid and aqueous NaCl. The mixture was concentrated to dryness at not more than 40° C. Dichloromethane was added and the mixture was filtered to give a solution of crude mcMMAF which was purified by column chromatography (not less than 52.7 kg of silica gel/kg of (5)) using dichloromethane as the loading solvent and eluting with 2-propanol in dichloromethane. Product containing fractions were analyzed by HPLC and selected based on fraction mapping (in-process control). The selected fractions were combined and evaporated to dryness at not more than 40° C. The product was dissolved in dichloromethane and evaporated to dryness. The product was sieved and dried at not more than 50° C. Typical yield was 47-75% of theory.

¹H NMR (750 MHz, DMSO-d6, 25 C): δ 12.74 (br s, 1H), 8.55 (br, 0.1H), 8.54 (d, 0.4H), 8.36 (dd, 0.5H), 8.13 (dd, 0.5H), 7.74 (d, 0.2H), 7.63 (d, 0.2H), 7.17-7.25 (m, 4.0H), 7.13.-7.17 (m, 1.0H), 7.02-6.99 (m, 1.7H), 4.80-4.67 (m, 0.5H), 4.67-4.58 (m, 1.4H), 4.55 (t, 0.3H), 4.51 (t, 0.3H), 4.46-4.41 (m, 0.8H), 4.41-4.38 (t, 0.2H), 4.03-3.99 (d, 0.4H), 3.99-3.93 (m, 1.2H), 3.76-3.73 (m, 0.5H), 3.57-3.51 (m, 0.7H), 3.49-3.45 (m, 0.8H), 3.45-3.41 (m, 0.9H), 3.40-3.35 (q, 2.4H), 3.31-3.27 (d, 0.7H), 3.24 (m, 1.6H), 3.23-3.15 (m, 6.2), 3.12-3.08 (dd, 0.6H), 3.05 (d, 1.6H), 2.99-3.03 (m, 0.6H), 2.97 (d, 1.4H), 2.91 (d, 1.3H), 2.78-2.86 (m, 2.9H), 2.48-2.37 (m, 1.1H), 2.37-2.25 (m, 2.0H), 2.25-2.20 (m, 1.7H), 2.05-2.20 (m, 1.7H), 2.05-1.90 (m, 1.2H), 1.87-1.70 (m, 2.1H), 1.70-1.59 (m, 1.1H), 1.55-1.45 (m, 4.5H), 1.45-1.39 (m, 1.2H), 1.39-1.33 (m, 0.6H), 1.33-1.26 (m, 1.0H), 1.26-1.18 (m, 2.7H), 1.09-1.04 (d, 1.7H), 1.04-1.00 (d, 1.6H), 0.95-0.89 (m, 2.6H), 0.89-0.85 (m, 4.0H), 0.85-0.79 (m, 7.0H), 0.79-0.74 (m 5.0H), 0.74-0.70 (t, 1.5H). LC/MS ESI+ [M+H]⁺ Calculated for C₄₉H₇₇N₆O₁₁ 925.5650, found: 925.5638.

Volumes and molar equivalents were expressed relative to the quantity of (5).

All publications, patent applications, patents, and other references mentioned herein are expressly incorporated by reference in their entireties, to the same extent as if each were incorporated by reference individually.

1. A process for making maleimidocaproyl monomethyl auristatin F comprising:
(a) converting compound 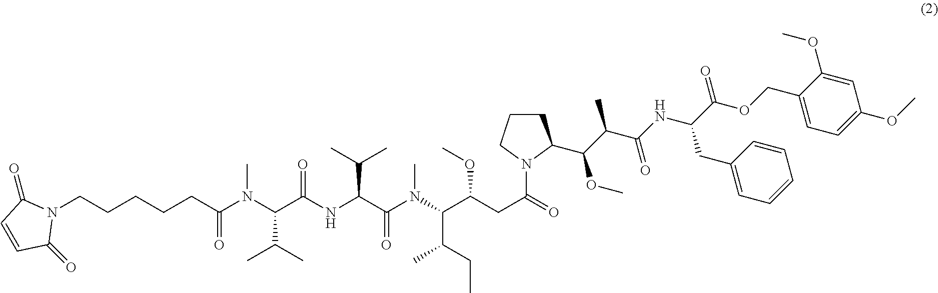 [SMILES: CC[C@H](C)[C@@H]([C@@H](CC(=O)N1CCC[C@H]1[C@H](OC)[C@@H](C)C(=O)N[C@@H](Cc1ccccc1)C(=O)OCc1ccc(OC)cc1OC)OC)N(C)C(=O)[C@@H](NC(=O)[C@H](C(C)C)N(C)C(=O)CCCCCN1C(=O)C=CC1=O)C(C)C]
to maleimidocaproyl monomethyl Auristatin F in the presence of toluene and 2-methyltetrahydrofuran,
(b) an aqueous workup, and
(c) chromatographic purification.

2. The process of claim 1, wherein 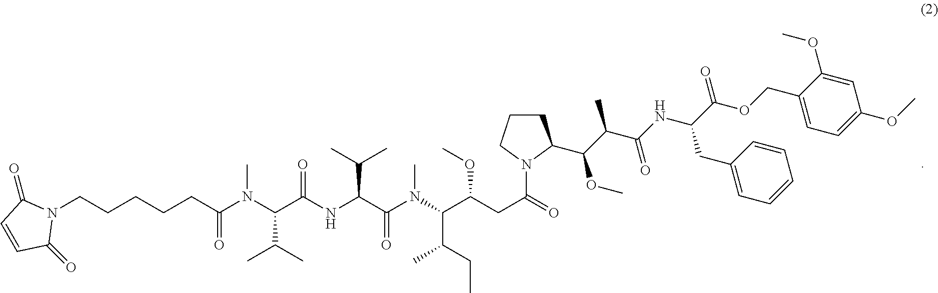 [SMILES: CC[C@H](C)[C@@H]([C@@H](CC(=O)N1CCC[C@H]1[C@H](OC)[C@@H](C)C(=O)N[C@@H](Cc1ccccc1)C(=O)OCc1ccc(OC)cc1OC)OC)N(C)C(=O)[C@@H](NC(=O)[C@H](C(C)C)N(C)C(=O)CCCCCN1C(=O)C=CC1=O)C(C)C]
is treated with an acid.

3. The process of claim 2, wherein said acid is trifluoroacetic acid.

4. The process of claim 1, further comprising a process for making 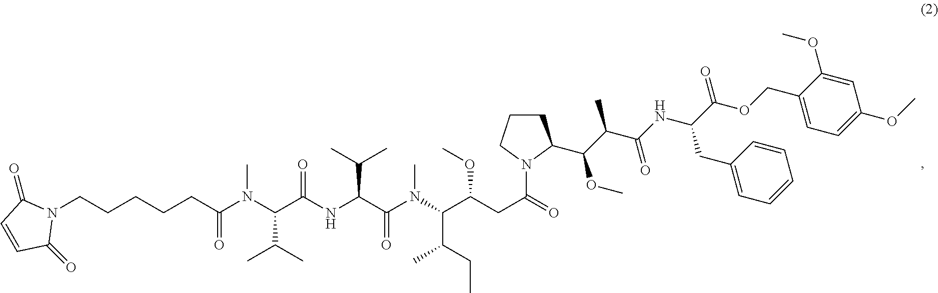 [SMILES: CC[C@H](C)[C@@H]([C@@H](CC(=O)N1CCC[C@H]1[C@H](OC)[C@@H](C)C(=O)N[C@@H](Cc1ccccc1)C(=O)OCc1ccc(OC)cc1OC)OC)N(C)C(=O)[C@@H](NC(=O)[C@H](C(C)C)N(C)C(=O)CCCCCN1C(=O)C=CC1=O)C(C)C]
wherein said process comprises reacting

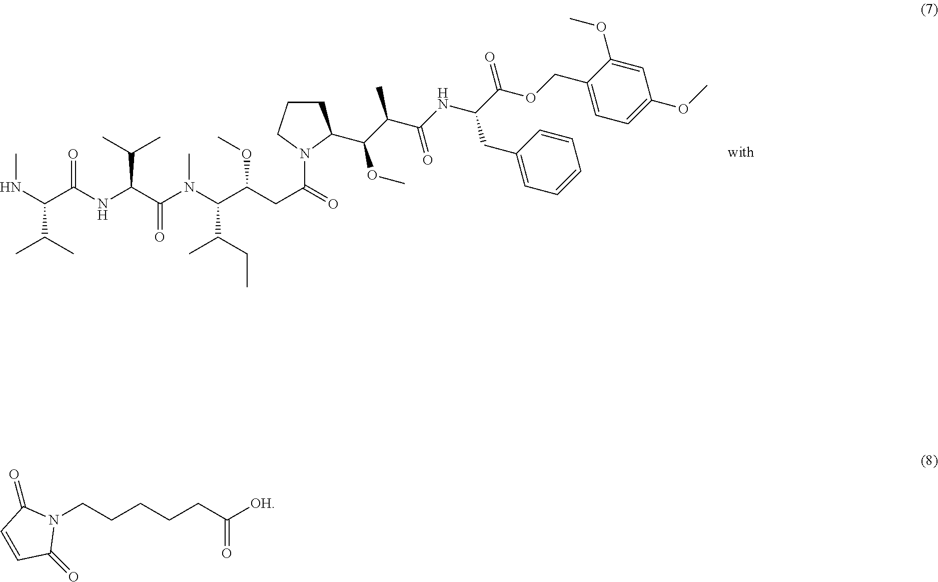 [SMILES: CCC(C)[C@@H]([C@@H](CC(=O)N1CCC[C@H]1[C@H](OC)[C@@H](C)C(=O)N[C@@H](Cc1ccccc1)C(=O)OCc1ccc(OC)cc1OC)OC)N(C)C(=O)[C@@H](NC(=O)[C@@H](NC)C(C)C)C(C)C ; O=C(O)CCCCCN1C(=O)C=CC1=O]

5. The process of claim 4, wherein 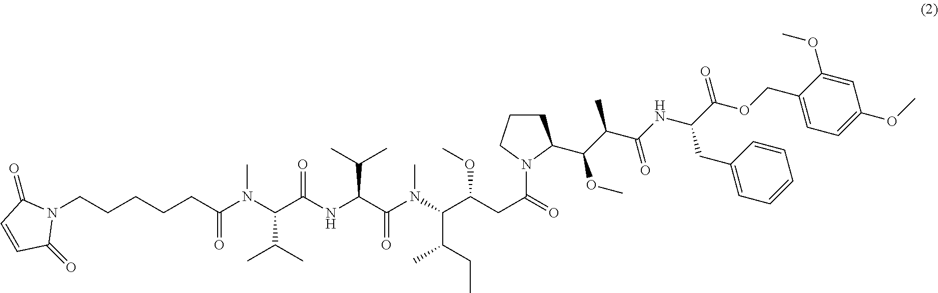 [SMILES: CC[C@H](C)[C@@H]([C@@H](CC(=O)N1CCC[C@H]1[C@H](OC)[C@@H](C)C(=O)N[C@@H](Cc1ccccc1)C(=O)OCc1ccc(OC)cc1OC)OC)N(C)C(=O)[C@@H](NC(=O)[C@H](C(C)C)N(C)C(=O)CCCCCN1C(=O)C=CC1=O)C(C)C]
is not isolated using column chromatography.

6. The process of claim 4, wherein reacting (7) with (8) occurs in the presence of a tertiary amine base.

7. The process of claim 6, wherein said base is N,N-diisopropylethylamine.

8-9. (canceled)

10. The process of claim 1, further comprising a process for making 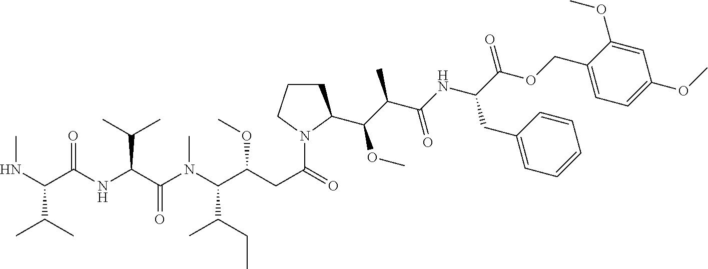 [SMILES: CCC(C)[C@@H]([C@@H](CC(=O)N1CCC[C@H]1[C@H](OC)[C@@H](C)C(=O)N[C@@H](Cc1ccccc1)C(=O)OCc1ccc(OC)cc1OC)OC)N(C)C(=O)[C@@H](NC(=O)[C@@H](NC)C(C)C)C(C)C]
(7), wherein said process comprises reacting 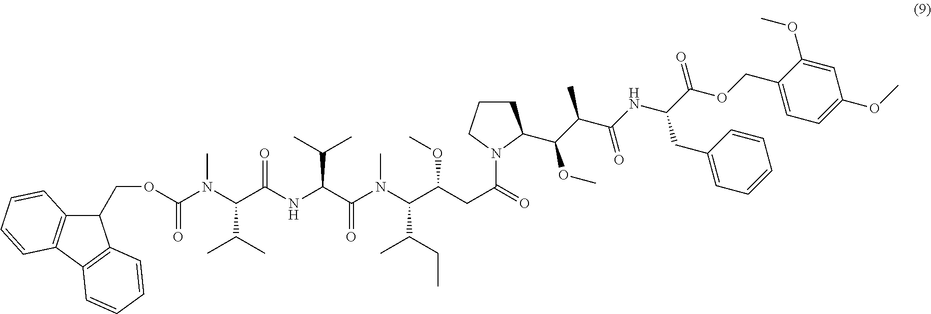 [SMILES: CCC(C)[C@@H]([C@@H](CC(=O)N1CCC[C@H]1[C@H](OC)[C@@H](C)C(=O)N[C@@H](Cc1ccccc1)C(=O)OCc1ccc(OC)cc1OC)OC)N(C)C(=O)[C@@H](NC(=O)[C@H](C(C)C)N(C)C(=O)OCC1c2ccccc2-c2ccccc21)C(C)C]
in the presence of a catalytic amount of DBU.

11. (canceled)

12. The process of claim 10, further comprising a process for making 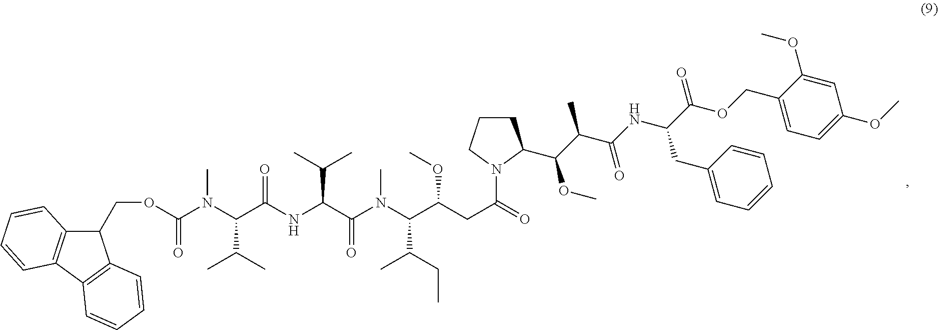 [SMILES: CCC(C)[C@@H]([C@@H](CC(=O)N1CCC[C@H]1[C@H](OC)[C@@H](C)C(=O)N[C@@H](Cc1ccccc1)C(=O)OCc1ccc(OC)cc1OC)OC)N(C)C(=O)[C@@H](NC(=O)[C@H](C(C)C)N(C)C(=O)OCC1c2ccccc2-c2ccccc21)C(C)C]
wherein said process comprises reacting 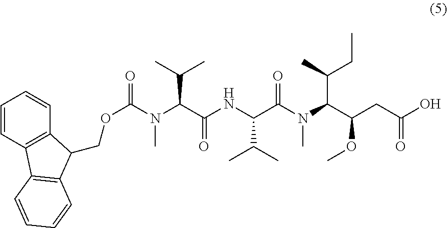 [SMILES: CC[C@H](C)[C@@H]([C@@H](CC(=O)O)OC)N(C)C(=O)[C@@H](NC(=O)[C@H](C(C)C)N(C)C(=O)OCC1c2ccccc2-c2ccccc21)C(C)C]
with the HCl salt of

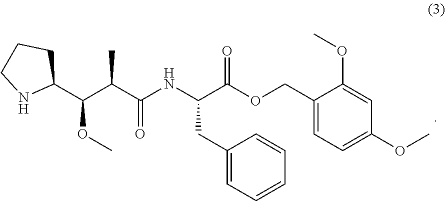 [SMILES: COc1ccc(COC(=O)[C@H](Cc2ccccc2)NC(=O)[C@H](C)[C@@H](OC)[C@@H]2CCCN2)c(OC)c1]

13-17. (canceled)

18. The process of claim 12, further comprising a process for making the HCl salt of 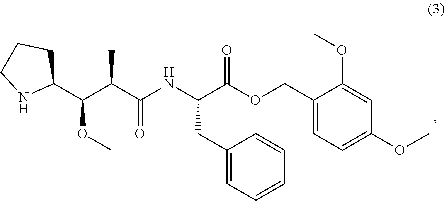 [SMILES: COc1ccc(COC(=O)[C@H](Cc2ccccc2)NC(=O)[C@H](C)[C@@H](OC)[C@@H]2CCCN2)c(OC)c1]
wherein said process comprises:
(a) reacting 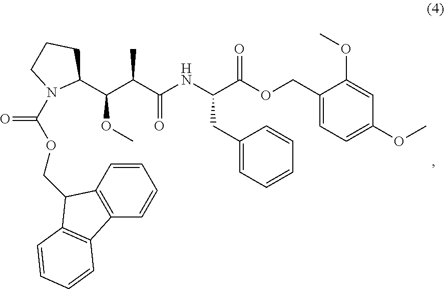 [SMILES: COc1ccc(COC(=O)[C@H](Cc2ccccc2)NC(=O)[C@H](C)[C@@H](OC)[C@@H]2CCCN2C(=O)OCC2c3ccccc3-c3ccccc32)c(OC)c1]
in the presence of a catalytic amount of DBU, and
(b) isolating 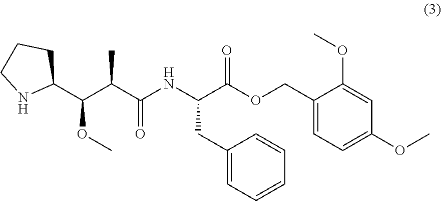 [SMILES: COc1ccc(COC(=O)[C@H](Cc2ccccc2)NC(=O)[C@H](C)[C@@H](OC)[C@@H]2CCCN2)c(OC)c1]
as a salt.

19-23. (canceled)

24. The process of claim 18, further comprising a process for making 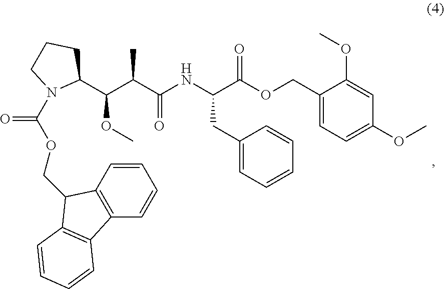 [SMILES: COc1ccc(COC(=O)[C@H](Cc2ccccc2)NC(=O)[C@H](C)[C@@H](OC)[C@@H]2CCCN2C(=O)OCC2c3ccccc3-c3ccccc32)c(OC)c1]
wherein said process comprises reacting the HCl salt of 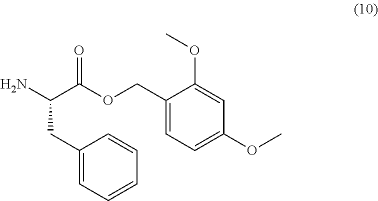 [SMILES: COc1ccc(COC(=O)[C@@H](N)Cc2ccccc2)c(OC)c1]
with

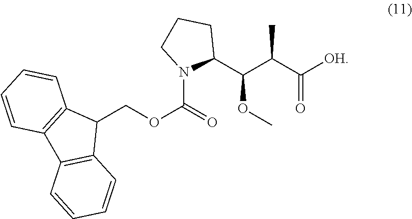 [SMILES: CO[C@H]([C@@H](C)C(=O)O)[C@@H]1CCCN1C(=O)OCC1c2ccccc2-c2ccccc21]

25-29. (canceled)

30. The process of claim 24, further comprising a process for making 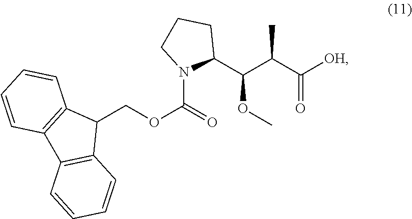 [SMILES: CO[C@H]([C@@H](C)C(=O)O)[C@@H]1CCCN1C(=O)OCC1c2ccccc2-c2ccccc21]
wherein said process comprises reacting the HCl salt of 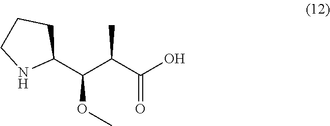
in the presence of water and dimethylformamide.

31-33. (canceled)

34. The process of claim 30, further comprising a process for making the HCl salt of 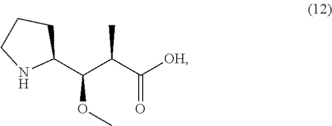
wherein said process comprises:
(a) contacting the DCHA salt of 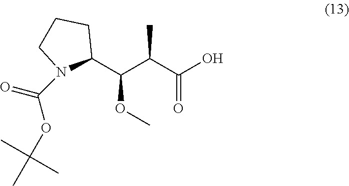 [SMILES: CO[C@H]([C@@H](C)C(=O)O)[C@@H]1CCCN1C(=O)OC(C)(C)C]
with an acid, and
(b) isolating 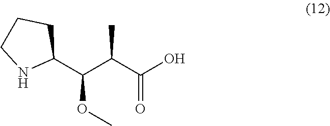
as an HCl salt.

35-36. (canceled)

37. The process of claim 34, further comprising a process for making 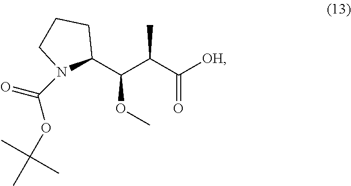 [SMILES: CO[C@H]([C@@H](C)C(=O)O)[C@@H]1CCCN1C(=O)OC(C)(C)C]
wherein said process comprises converting 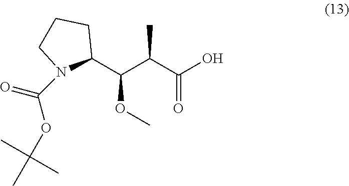 [SMILES: CO[C@H]([C@@H](C)C(=O)O)[C@@H]1CCCN1C(=O)OC(C)(C)C]
DCHA into

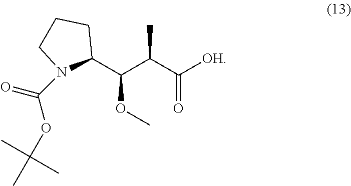 [SMILES: CO[C@H]([C@@H](C)C(=O)O)[C@@H]1CCCN1C(=O)OC(C)(C)C]

38-41. (canceled)

42. The process of claim 24, further comprising a process for making 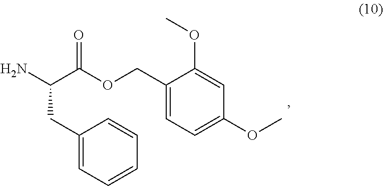 [SMILES: COc1ccc(COC(=O)[C@@H](N)Cc2ccccc2)c(OC)c1]
wherein said process comprises:
(a) reacting 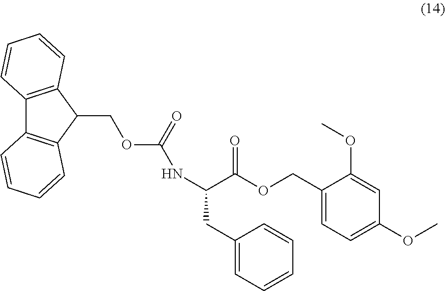 [SMILES: COc1ccc(COC(=O)[C@H](Cc2ccccc2)NC(=O)OCC2c3ccccc3-c3ccccc32)c(OC)c1]
in the presence of a catalytic amount of DBU, and
(b) isolating 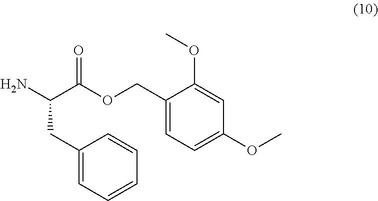 [SMILES: COc1ccc(COC(=O)[C@@H](N)Cc2ccccc2)c(OC)c1]
as an HCl salt.

43-46. (canceled)

47. The process of claim 42, further comprising a process for making 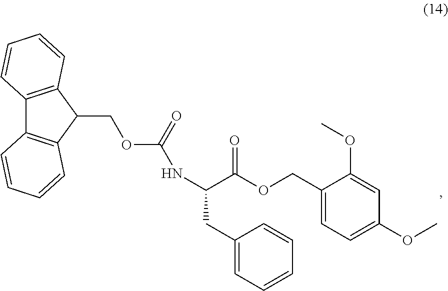 [SMILES: COc1ccc(COC(=O)[C@H](Cc2ccccc2)NC(=O)OCC2c3ccccc3-c3ccccc32)c(OC)c1]
wherein said process comprises reacting 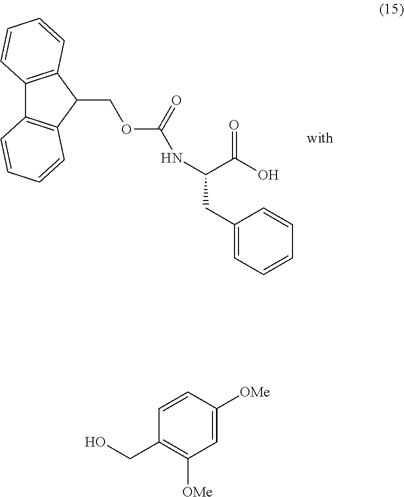 [SMILES: O=C(N[C@@H](Cc1ccccc1)C(=O)O)OCC1c2ccccc2-c2ccccc21 ; COc1ccc(CO)c(OC)c1]
in a solvent other than dichloromethane.

48-53. (canceled)

54. The process of claim 12, further comprising a process for making 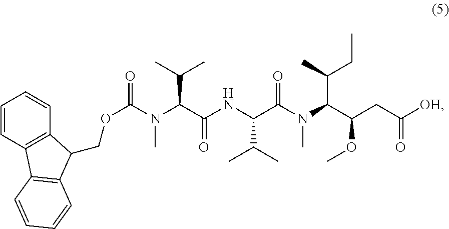 [SMILES: CC[C@H](C)[C@@H]([C@@H](CC(=O)O)OC)N(C)C(=O)[C@@H](NC(=O)[C@H](C(C)C)N(C)C(=O)OCC1c2ccccc2-c2ccccc21)C(C)C]
wherein said process comprises:
(a) reacting 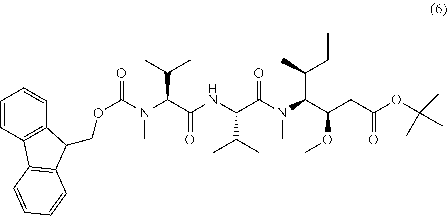 [SMILES: CC[C@H](C)[C@@H]([C@@H](CC(=O)OC(C)(C)C)OC)N(C)C(=O)[C@@H](NC(=O)[C@H](C(C)C)N(C)C(=O)OCC1c2ccccc2-c2ccccc21)C(C)C]
in the presence of aqueous sulfuric acid, and
(b) isolating 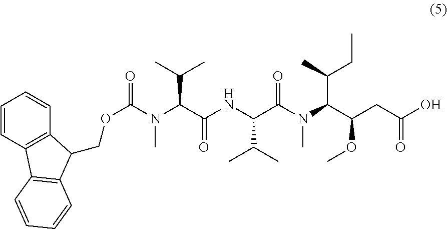 [SMILES: CC[C@H](C)[C@@H]([C@@H](CC(=O)O)OC)N(C)C(=O)[C@@H](NC(=O)[C@H](C(C)C)N(C)C(=O)OCC1c2ccccc2-c2ccccc21)C(C)C]
without a workup.

55. (canceled)

56. The process of claim 54, further comprising a process for making 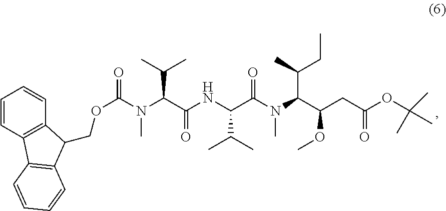 [SMILES: CC[C@H](C)[C@@H]([C@@H](CC(=O)OC(C)(C)C)OC)N(C)C(=O)[C@@H](NC(=O)[C@H](C(C)C)N(C)C(=O)OCC1c2ccccc2-c2ccccc21)C(C)C]
wherein said process comprises reacting 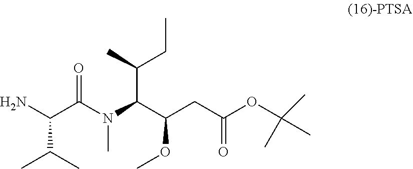 [SMILES: CC[C@H](C)[C@@H]([C@@H](CC(=O)OC(C)(C)C)OC)N(C)C(=O)[C@@H](N)C(C)C]
with Fmoc-NmeVal-OH.

57-61. (canceled)

62. The process of claim 56, further comprising a process for making 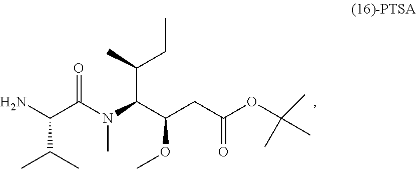 [SMILES: CC[C@H](C)[C@@H]([C@@H](CC(=O)OC(C)(C)C)OC)N(C)C(=O)[C@@H](N)C(C)C]
wherein said process comprises
(a) hydrogenating 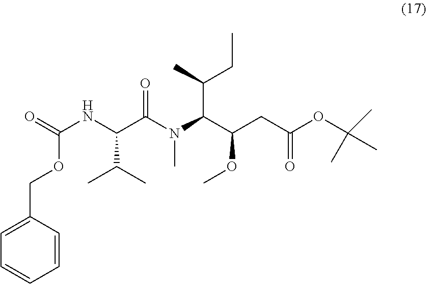 [SMILES: CC[C@H](C)[C@@H]([C@@H](CC(=O)OC(C)(C)C)OC)N(C)C(=O)[C@@H](NC(=O)OCc1ccccc1)C(C)C]
in the presence of a catalyst,
(b) reacting the product of step (a) with PTSA monohydrate in the presence of 2-methyltetrahydrofuran,
(c) adding 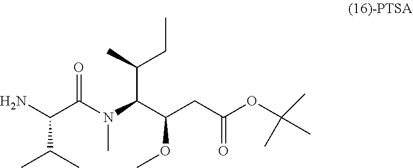 [SMILES: CC[C@H](C)[C@@H]([C@@H](CC(=O)OC(C)(C)C)OC)N(C)C(=O)[C@@H](N)C(C)C]
seed, and
(d) isolating

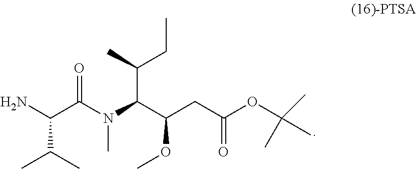 [SMILES: CC[C@H](C)[C@@H]([C@@H](CC(=O)OC(C)(C)C)OC)N(C)C(=O)[C@@H](N)C(C)C]

63-64. (canceled)

65. The process of claim 62, further comprising a process for making 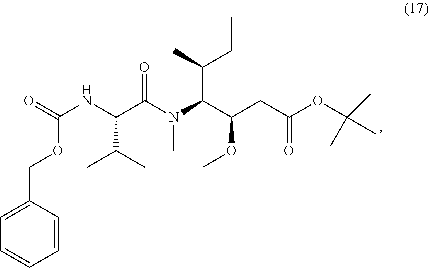 [SMILES: CC[C@H](C)[C@@H]([C@@H](CC(=O)OC(C)(C)C)OC)N(C)C(=O)[C@@H](NC(=O)OCc1ccccc1)C(C)C]
wherein said process comprises reacting 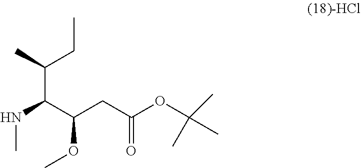 [SMILES: CC[C@H](C)[C@H](NC)[C@@H](CC(=O)OC(C)(C)C)OC]
with Z-Val-OH in the presence of Surfactant TPGS-750-M.

66-71. (canceled)
